(19) United States
(12) Patent Application Publication
(10) Pub. No.: US 2024/0326030 A1
(43) Pub. Date: Oct. 3, 2024

(54) PALLADIUM COMPLEX AND CATALYST EMBODIMENTS AND METHODS OF MAKING AND USING THE SAME

(71) Applicants: David LEITCH, Victoria (CA); Jingjun hUANG, Victoria (CA); Matthew ISAAC, Victoria (CA); Ryan WATT, Victoria (CA); UVic Industry Partnerships Inc., Victoria (CA)

(72) Inventors: David Leitch, Victoria (CA); Jingjun Huang, Victoria (CA); Matthew Isaac, Victoria (CA); Ryan Watt, Victoria (CA)

(73) Assignee: UVic Industry Partnerships Inc., Victoria, BC (CA)

(21) Appl. No.: 18/271,800

(22) PCT Filed: Jan. 11, 2022
(86) PCT No.: PCT/IB2022/050196

§ 371 Date: Jul. 11, 2023

Related U.S. Application Data
(60) Provisional application No. 63/136,456, filed on Jan. 12, 2021.

(51) Int. Cl. B01J 31/18; B01J 31/22; B01J 31/24; C07C 45/64; C07C 67/343; C07C 303/40; C07D 213/74; C07D 235/00; C07D 295/096

(52) U.S. Cl. CPC B01J 31/1815; B01J 31/2295; B01J 31/2409; C07C 45/64; C07C 67/343; C07C 303/40; C07D 213/74; C07D 235/00; C07D 295/096; B01J 2231/4261; B01J 2231/4283; B01J 2231/4288; B01J 2531/0241; B01J 2531/824

(57) ABSTRACT

Disclosed herein are embodiments of a Pd(0) precursor complex and embodiments of phosphorus-based Pd(0) catalysts formed therefrom. Also disclosed are method embodiments for making the Pd(0) precursor complex and the phosphorus-based Pd(0) catalysts. The Pd(0) precursor complex can be used to generate, in situ, the phosphorus-based Pd(0) catalysts, in various different types of palladium-mediated coupling reactions.

### CROSS REFERENCE TO RELATED APPLICATIONS

This application claims the benefit of and priority to the earlier filing date of U.S. Provisional Patent Application No. 63/136,456, filed on Jan. 12, 2021; this prior application is incorporated herein by reference in its entirety.

### FIELD

The present disclosure concerns palladium catalyst embodiments and methods of making and using the same.

### BACKGROUND

Most cross-coupling requires the aid of a transition metal catalyst to proceed. Pd(0)-containing catalysts are the most widely used and studied. Early methods of generating Pd(0) catalysts employed Pd(II) salts, such as palladium dichloride and palladium acetate, as precatalysts; however, the reduction pathway involved with such catalysts can be unreliable and is often very condition-dependent. Another method is to directly use a Pd(0)Lx complex as a precatalyst, such as Pd(PPh₃)₄ or bis(dibenzylideneacetone)palladium(0) (or “Pd(dba)₂”); however, these compounds also come with drawbacks. For example, Pd(0)-dba complexes can produce inconsistent catalytic results depending on source, degrade rapidly in solution to produce palladium nanoparticles (palladium black), and the dba released after activation is “non-innocent” and can interfere with catalysis. Pd(II)-containing precatalysts exist; however, such precatalysts still require basic conditions to be activated, and the pre-installation of the phosphine ligand makes each precatalyst specific to the substrates being coupled. And, not every phosphine that could be used in cross-coupling is available as part of a precatalyst. There exists a need in the art for a universal Pd(0) precursor able to coordinate a wide array of phosphine ligands and directly enter the cross-coupling catalytic cycle.

### SUMMARY

Disclosed herein are embodiments of a complex, having a structure according to Formula III

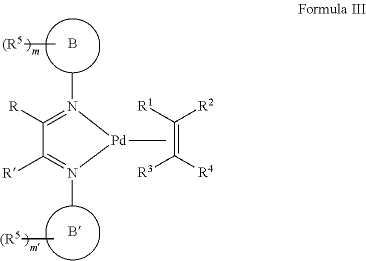

wherein each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; each of R and R′ independently is selected from hydrogen, aliphatic, heteroaliphatic, aromatic, or an organic functional group, or R and R′ together to provide an aromatic, heterocyclic, or alicyclic ring system; each of the B ring and B′ ring independently is an aromatic ring system; each R⁵ independently is selected from aliphatic, heteroaliphatic, haloaliphatic, aromatic an organic functional group, or a combination thereof; each R⁶ independently is selected from aliphatic, heteroaliphatic, haloaliphatic, aromatic an organic functional group, or a combination thereof; and each of m and m′ independently is an integer selected from 0 to 10.

Also disclosed herein are embodiments of a catalyst, having a structure according to Formula IV, VA, or VB

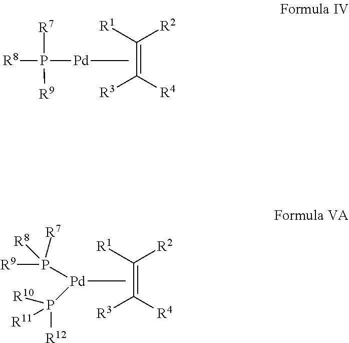

wherein each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; each of R⁷, R⁸, R⁹, R¹⁰, R¹¹, and R¹² independently is selected from aliphatic, heteroaliphatic, haloaliphatic, haloheteroaliphatic, aromatic, or an organic functional group; or two or more of R⁷, R⁸, and R⁹, and/or independently two or more of R¹⁰, R¹¹, and R¹², can join together, with the phosphorus atom to which they are attached, to provide a heterocyclic ring system

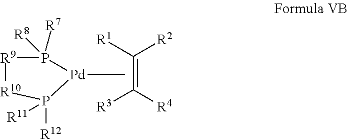

wherein each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; or each of R⁷, R⁸, R¹¹, and R¹² independently is selected from aliphatic, heteroaliphatic, haloaliphatic, haloheteroaliphatic, aromatic, or an organic functional group and R⁹ and R¹⁰ independently are aliphatic, heteroaliphatic, haloaliphatic, haloheteroaliphatic, aromatic, or an organic functional group and are bound together through one or more carbon atoms and/or heteroatoms.

Also disclosed are method embodiments for making the complex embodiments of the present disclosure, comprising exposing a Pd₂dba₃ complex to a ligand compound comprising a N,N-substituted diazabutadiene group to form a reaction mixture.

Also disclosed are embodiments of a method of making a catalyst embodiment of the present disclosure, comprising exposing a phosphorus-containing ligand group to a complex having a structure according to Formula III, described above.

Also disclosed herein are embodiments of a method, comprising using a complex or catalyst according to the present disclosure as a catalyst in a palladium-mediated coupling reaction.

The foregoing and other objects and features of the present disclosure will become more apparent from the following detailed description, which proceeds with reference to the accompanying figures.

### BRIEF DESCRIPTION OF THE DRAWINGS

FIGS. 1A-1C show characterization spectra for a representative Pd(0) precursor complex embodiment, ^{DMP}DAB-Pd-MAH (complex 1), wherein FIG. 1A is a ¹H-NMR spectrum; FIG. 1B is a ¹³C-NMR spectrum, and FIG. 1C is a mass spectrum.

FIGS. 2A and 2B show solid state structures of representative Pd(0) precursor complex embodiments, wherein FIG. 2A shows the solid state structure for ^{DMP}DAB-Pd-MAH (complex 1) and FIG. 2B shows the solid state structure for bis(N,N′-Bis(2,6-diisopropylphenyl)ethan-1,2-diimine)(η₂-furan-2,5-dione)palladium(0).

FIGS. 3A and 3B show ¹H NMR spectra (300 MHz) stack plots for solution stability of ^{DMP}DAB-Pd-MAH in CDCl₃ (FIG. 3A) and d₆-THF (FIG. 3B) with time increasing from front (30 minutes) to back (48 hours).

FIG. 4 is a graph showing a summary of the solution stability of ^{DMP}DAB-Pd-MAH at 20 mg/mL concentration in five deuterated solvents over 48 hours at room temperature.

FIG. 5 is a graph showing a comparison of ligand substitution reaction progress between ^{DMP}DAB-Pd-MAH (complex 1) and several phosphines (P:Pd=2) to generate [ligand]-Pd-MAH complexes, wherein both DPEPhos and Bippyphos result in >95% substitution in less than 5 minutes; NMR yields determined by relative integration of product signals to 1,3,5-trimethoxybenzene internal standard.

FIGS. 6A-6D show characterization spectra for a representative phosphorus-based Pd(0) catalyst (catalyst 2) wherein FIG. 6A is a ¹H-NMR spectrum; FIG. 6B is a ³¹P-NMR spectrum; FIG. 6C is a ¹³C-NMR spectrum, and FIG. 6D is a mass spectrum.

FIGS. 7A-7D show characterization spectra for a representative phosphorus-based Pd(0) catalyst (catalyst 3) wherein FIG. 7A is a ¹H-NMR spectrum; FIG. 7B is a ³¹P-NMR spectrum; FIG. 7C is a ¹³C-NMR spectrum, and FIG. 7D is a mass spectrum.

FIGS. 8A and 8B show solid state structures for catalyst 3, wherein FIG. 8B omits hydrogen atoms (except for those on the maleic anhydride moiety) and shows the groups attached to the phosphorus atoms in wireframe for simplicity.

FIGS. 9A-9D show characterization spectra for a representative phosphorus-based Pd(0) catalyst (catalyst 4) wherein FIG. 9A is a ¹H-NMR spectrum; FIG. 9B is a ³¹P-NMR spectrum; FIG. 9C is a ¹³C-NMR spectrum, and FIG. 9D is a mass spectrum.

FIGS. 10A and 10B show solid state structures for catalyst 4, wherein FIG. 10B omits hydrogen atoms (except for those on the maleic anhydride moiety) and shows the groups attached to the phosphorus atoms in wireframe for simplicity.

FIGS. 11A-11D show characterization spectra for a representative phosphorus-based Pd(0) catalyst (catalyst 5) wherein FIG. 11A is a ¹H-NMR spectrum; FIG. 11B is a ³¹P-NMR spectrum; FIG. 11C is a ¹³C-NMR spectrum, and FIG. 11D is a mass spectrum.

FIGS. 12A and 12B show solid state structures for catalyst 5, wherein FIG. 12B omits hydrogen atoms (except for those on the maleic anhydride moiety) and shows the groups attached to the phosphorus atoms in wireframe for simplicity.

FIGS. 13A-13D show characterization spectra for a representative phosphorus-based Pd(0) catalyst (catalyst 6) wherein FIG. 13A is a ¹H-NMR spectrum; FIG. 13B is a ³¹P-NMR spectrum; FIG. 13C is a ¹³C-NMR spectrum, and FIG. 13D is a mass spectrum.

FIGS. 14A-14D show characterization spectra for a representative phosphorus-based Pd(0) catalyst (catalyst 7) wherein FIG. 14A is a ¹H-NMR spectrum; FIG. 14B is a ³¹P-NMR spectrum; FIG. 14C is a ¹³C-NMR spectrum, and FIG. 14D is a mass spectrum.

FIGS. 15A-15D show characterization spectra for a representative phosphorus-based Pd(0) catalyst (catalyst 8) wherein FIG. 15A is a ¹H-NMR spectrum; FIG. 15B is a ³¹P-NMR spectrum; FIG. 15C is a ¹³C-NMR spectrum, and FIG. 15D is a mass spectrum.

FIGS. 16A and 16B show solid state structures for catalyst 8, wherein FIG. 16B omits hydrogen atoms (except for those on the maleic anhydride moiety) and shows the groups attached to the phosphorus atom in wireframe for simplicity.

FIGS. 17A-17D show characterization spectra for a representative phosphorus-based Pd(0) catalyst (catalyst 9) wherein FIG. 17A is a ¹H-NMR spectrum; FIG. 17B is a ³¹P-NMR spectrum; FIG. 17C is a ¹³C-NMR spectrum, and FIG. 17D is a mass spectrum.

FIGS. 18A-18D show characterization spectra for a representative phosphorus-based Pd(0) catalyst (catalyst 10) wherein FIG. 18A is a ¹H-NMR spectrum; FIG. 18B is a ³¹P-NMR spectrum; FIG. 18C is a ¹³C-NMR spectrum, and FIG. 18D is a mass spectrum.

FIGS. 19A-19D show characterization spectra for a representative phosphorus-based Pd(0) catalyst (catalyst 11) wherein FIG. 19A is a ¹H-NMR spectrum; FIG. 19B is a ³¹P-NMR spectrum; FIG. 19C is a ¹³C-NMR spectrum, and FIG. 19D is a mass spectrum.

FIGS. 20A-20D show characterization spectra for a representative phosphorus-based Pd(0) catalyst (catalyst 12) wherein FIG. 20A is a ¹H-NMR spectrum; FIG. 20B is a ³¹P-NMR spectrum; FIG. 20C is a ¹³C-NMR spectrum, and FIG. 20D is a mass spectrum.

FIGS. 21A and 21B show solid state structures for catalyst 12, wherein FIG. 21B omits hydrogen atoms (except for those on the maleic anhydride moiety) and shows the groups attached to the phosphorus atom in wireframe for simplicity.

FIGS. 22A and 22B show results from comparing ligand substitution reaction progress between ^{DMP}DAB-Pd-MAH (complex 1) or ^{tBu}DAB-Pd-MAH and Me₄tBuXPhos; FIG. 22A is a graph of % starting complex as a function of time (minutes); FIG. 22B is an ¹H NMR spectrum stack plot of the ligand substitution between ^{DMP}DAB-Pd-MAH (complex 1) (singlet at 8.24 ppm) and Me₄tBuXPhos (2 equivalents), with time increasing from front (0 minutes) to back (62 minutes).

FIGS. 23A-23D show characterization spectra for a representative phosphorus-based Pd(0) catalyst (catalyst 13) wherein FIG. 23A is a ¹H-NMR spectrum; FIG. 23B is a ³¹P-NMR spectrum; FIG. 23C is a ¹³C-NMR spectrum, and FIG. 23D is a mass spectrum.

FIGS. 24A-24E show characterization spectra for a representative phosphorus-based Pd(0) catalyst (catalyst 14) wherein FIG. 24A is a ¹H-NMR spectrum; FIG. 24B is a ³¹P-NMR spectrum; FIG. 24C is a ¹⁹F-NMR spectrum; FIG. 24D ¹³C-NMR spectrum, and FIG. 24E is a mass spectrum.

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and complexes and/or catalysts similar or equivalent to those described herein can be used in the practice or testing of the present disclosure, suitable methods and complexes and/or catalysts are described below. The complexes and/or catalysts, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Complex and/or catalyst embodiments disclosed herein may contain one or more asymmetric elements such as stereogenic centers, stereogenic axes and the like, e.g., asymmetric carbon atoms, so that the chemical conjugates can exist in different stereoisomeric forms. These complex and/or catalyst embodiments can be, for example, racemates or optically active forms. For complex and/or catalyst with two or more asymmetric elements, these complex and/or catalyst can additionally be mixtures of diastereomers. For complex and/or catalyst having asymmetric centers, all optical isomers in pure form and mixtures thereof are encompassed by corresponding generic formulas unless context clearly indicates otherwise or an express statement excluding an isomer is provided. In these situations, the single enantiomers, i.e., optically active forms can be obtained by method known to a person of ordinary skill in the art, such as asymmetric synthesis, synthesis from optically pure precursors, or by resolution of the racemates. Resolution of the racemates can also be accomplished, for example, by conventional methods, such as crystallization in the presence of a resolving agent, or chromatography, using, for example a chiral HPLC column. All isomeric forms are contemplated herein regardless of the methods used to obtain them.

### A. Definitions

To facilitate review of the various embodiments of the disclosure, the following definitions and explanations of specific terms are provided. Certain functional group terms include a symbol “-” which is used to show how the defined functional group attaches to, or within, the disclosed complex and/or catalyst to which it is bound. Also, a dashed bond (i.e., [symbol] ), as used in certain formulas described herein, indicates an optional bond (that is, a bond that may or may not be present). A person of ordinary skill in the art would recognize that the definitions provided below and complex and/or catalyst and formulas included herein are not intended to include impermissible substitution patterns (e.g., methyl substituted with 5 different groups, and the like). Such impermissible substitution patterns are easily recognized by a person of ordinary skill in the art. In formulas and complex and/or catalyst disclosed herein, a hydrogen atom is present and completes any formal valency requirements (but may not necessarily be illustrated) wherever a functional group or other atom is not illustrated on a carbon atom. For example, a phenyl ring that is drawn as

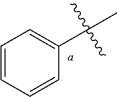 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Acyl Halide: —C(O)X, wherein X is a halogen, such as Br, F, I, or Cl.

Aldehyde: —C(O)H.

Alicyclic: A cyclic hydrocarbon group that can comprise one or more ring systems, including spirocyclic, bicyclic, and/or fused cyclic ring systems.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Aliphatic-aromatic: An aromatic group that is or can be coupled to a complex and/or catalyst disclosed herein, wherein the aromatic group is or becomes coupled through an aliphatic group.

Aliphatic-aryl: An aryl group that is or can be coupled to a complex and/or catalyst disclosed herein, wherein the aryl group is or becomes coupled through an aliphatic group.

Aliphatic-heteroaryl: A heteroaryl group that is or can be coupled to a complex and/or catalyst disclosed herein, wherein the heteroaryl group is or becomes coupled through an aliphatic group.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amide: —C(O)NR^{a}R^{b} or —NR^{a}C(O)R^{b} wherein each of R^{a} and R^{b} independently is selected from hydrogen, aliphatic, heteroaliphatic, haloaliphatic, haloheteroaliphatic, aromatic, or an organic functional group.

Amino: —NR^{a}R^{b}, wherein each of R^{a} and R^{b} independently is selected from hydrogen, aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

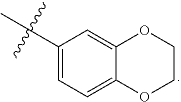 [SMILES: *c1ccc2c(c1)OCCO2]

However, in certain examples, context or express disclosure may indicate that the point of attachment is through a non-aromatic portion of the condensed ring system. For example,

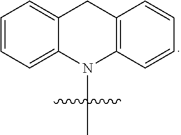 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the complexes and/or catalysts disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: —O-aromatic.

Azide: —N₃

Azo: —N═NR^{a} wherein R^{a} is hydrogen, aliphatic, heteroaliphatic, haloaliphatic, haloheteroaliphatic, aromatic, or an organic functional group.

Carbamate: —OC(O)NR^{a}R^{b}, wherein each of R^{a} and R^{b} independently is selected from hydrogen, aliphatic, heteroaliphatic, haloaliphatic, haloheteroaliphatic, aromatic, or an organic functional group.

Carboxyl: —C(O)OH.

Carboxylate: —C(O)O— or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Cyano: —CN.

Disulfide: —SSR^{a}, wherein R^{a} is selected from hydrogen, aliphatic, heteroaliphatic, haloaliphatic, haloheteroaliphatic, aromatic, or an organic functional group.

Dithiocarboxylic: —C(S)SR^{a} wherein R^{a} is selected from hydrogen, aliphatic, heteroaliphatic, haloaliphatic, haloheteroaliphatic, aromatic, or an organic functional group.

Electron-Withdrawing Group: A functional group capable of accepting electron density from an aromatic ring or olefin moiety to which it is directly attached, such as by inductive electron withdrawal.

Representative and non-limiting examples of electron-withdrawing groups can include certain heteroaliphatic groups (e.g., aldehyde, ketone, ester, carboxylic acid, acyl, a quaternary amine, acyl halide, cyano, sulfonate, nitro, nitroso, pyridinyl, alkyl halide, halogen (e.g., chloro, bromo, fluoro, or iodo), haloaliphatic, ammonium, or amide.

Ester: —C(O)OR^{a} or —OC(O)R^{a}, wherein R^{a} is selected from aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloaliphatic-aryl: An aryl group that is or can be coupled to a complex and/or catalyst disclosed herein, wherein the aryl group is or becomes coupled through a haloaliphatic group.

Haloaliphatic-heteroaryl: A heteroaryl group that is or can be coupled to a complex and/or catalyst disclosed herein, wherein the heteroaryl group is or becomes coupled through a haloaliphatic group.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. In some embodiments, a fluorophore can also be described herein as a heteroaliphatic group, such as when the heteroaliphatic group is a heterocyclic group.

Heteroaliphatic-aryl: An aryl group that is or can be coupled to a complex and/or catalyst disclosed herein, wherein the aryl group is or becomes coupled through a heteroaliphatic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group. In some embodiments, a fluorophore can also be described herein as a heteroaryl group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Heterocyclic: A heterocyclic group comprising one or more heteroatoms and that can comprise one or more ring systems, including spirocyclic, bicyclic, and/or fused cyclic ring systems.

Ketone: —C(O)R^{a}, wherein R^{a} is selected from aliphatic, heteroaliphatic, haloaliphatic, haloheteroaliphatic, aromatic, or an organic functional group.

Nitro: —NO₂

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic acid or ester; phosphonate; phosphate; silyl ether; sulfinyl; thial; or combinations thereof.

Oxime: —CR^{a}═NOH, wherein R^{a} is hydrogen, aliphatic, heteroaliphatic, haloaliphatic, haloheteroaliphatic, aromatic, or an organic functional group.

Peroxy: —O—OR^{a} wherein R^{a} is hydrogen, aliphatic, heteroaliphatic, haloaliphatic, haloheteroaliphatic, aromatic, or an organic functional group.

Phosphate: —O—P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Silyl Ether: —OSiR^{a}R^{b}, wherein each of R^{a} and R^{b} independently is selected from hydrogen, aliphatic, heteroaliphatic, haloaliphatic, haloheteroaliphatic, aromatic, or an organic functional group.

Sulfinyl: —S(O)R^{a}, wherein R^{a} is selected from hydrogen, aliphatic, heteroaliphatic, haloaliphatic, haloheteroaliphatic, aromatic, or an organic functional group.

Sulfonyl: —SO₂R^{a}, wherein R^{a} is selected from hydrogen, aliphatic, heteroaliphatic, haloaliphatic, haloheteroaliphatic, aromatic, or an organic functional group.

Sulfonamide: —SO₂NR^{a}R^{b} or —N(R^{a})SO₂R^{b}, wherein each of R^{a} and R^{b} independently is selected from hydrogen, aliphatic, heteroaliphatic, haloaliphatic, haloheteroaliphatic, aromatic, or an organic functional group.

Sulfonate: —SO₃⁻, wherein the negative charge of the sulfonate group may be balanced with an M⁺ counter ion, wherein M⁺ may be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5.}

Thial: —C(S)H.

Thiocarboxylic acid: —C(O)SH, or —C(S)OH.

Thiocyanate: —S—CN or —N═C═S.

Thioester: —C(O)SR^{a} or —C(S)OR^{a} wherein R^{a} is selected from hydrogen, aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S— heteroaryl; or -aliphatic-S-aliphatic, -aliphatic-S-aromatic, -aromatic-S-aliphatic, or -aromatic-S-aromatic.

Thioketone: —C(S)R^{a} wherein R^{a} is selected from hydrogen, aliphatic, heteroaliphatic, haloaliphatic, haloheteroaliphatic, aromatic, or an organic functional group.

### B. Abbreviations/Ligand Names

BippyPhos: 5-(Di-tert-butylphosphino)-1′, 3′, 5′-triphenyl-1′H-[1,4′]bipyrazole

BrettPhos: 2-(Dicyclohexylphosphino)3,6-dimethoxy-2′,4′,6′-triisopropyl-1,1′-biphenyl [097] cataCXium® A: Di(1-adamantyl)-n-butylphosphine

DAB: diazabutadiene

DMP: Dimethylphenyl

Dppf: 1,1′-Bis(diphenylphosphino)ferrocene

Dppp: 1,3-bis(diphenylphosphino)propane

DPEPhos: Bis[(2-diphenylphosphino)phenyl] ether

HTE: High-throughput experimentation

JackiePhos: 2-{Bis[3,5-bis(trifluoromethyl)phenyl]phosphino}-3,6-dimethoxy-2′,4′,6′-triisopropyl-1,1′-biphenyl

MAH: Maleic anhydride

Me₄tBuXPhos: 2-Di-tert-butylphosphino-3,4,5,6-tetramethyl-2′,4′,6′-triisopropyl-1,1′-biphenyl

RuPhos: 2-Dicyclohexylphosphino-2′,6′-diisopropoxybiphenyl

SPhos: 2-Dicyclohexylphosphino-2′,6′-dimethoxybiphenyl

tBuBrettPhos: 2-(Di-tert-butylphosphino)-2′,4′,6′-triisopropyl-3,6-dimethoxy-1,1′-biphenyl

tBuXPhos: 2-Di-tert-butylphosphino-2′,4′,6′-triisopropylbiphenyl

XantPhos: 4,5-Bis(diphenylphosphino)-9,9-dimethylxanthene

XPhos: 2-Dicyclohexylphosphino-2′,4′,6′-triisopropylbiphenyl

### II. Introduction

Palladium-catalyzed cross-coupling is among the most powerful tools in modern synthetic chemistry, with extensive applications in total synthesis, pharmaceutical discovery and process chemistry, agrochemistry, and materials chemistry. Given the large number of reaction variables to optimize for every new system, and the extraordinary array of reported reaction conditions, microscale high-throughput experimentation (HTE) is becoming an important tool for practical implementation of cross-coupling catalysis. This technique enables simultaneous exploration of multiple reaction variables using a minimum amount of material, providing a holistic picture of reaction systems and revealing interactions between variables that are not evident through one-variable-at-a-time studies.

As is true for nearly all homogeneous catalysts, ligand identity is one of the most important factors in cross-coupling reaction design. Accordingly, screening against a ligand library is a key step in both standard and high-throughput optimization workflows. One strategy is to use a precatalyst series, where the desired ligands are already metallated to Pd to form discrete complexes. While this ensures complete metallation and gives a fixed Pd/ligand ratio, it requires a large number of specific, individual Pd complexes to be available; but not every useful ligand is available as part of a precatalyst. In addition, the Pd/ligand ratio is often must be controlled and in some conventional systems, “extra” ligand is added to reactions involving precatalysts to improve catalyst stability, which can result in unwanted side-products and/or difficulty in isolating desired products. A more general and operationally simpler screening strategy is to combine the free ligand with an appropriate Pd precursor to generate the catalyst in situ. This is especially useful when exploring a large ligand library, and also can reveal unexpected catalyst activation effects; however, this also introduces additional variables and uncertainties, such as what Pd precursor should be used and how to ensure that metallation is successful.

Since the advent of Pd-catalyzed cross-coupling, Pd(II) precursors are the most common choice for in situ catalyst formation. Simple Pd(II) sources, such as halide and carboxylate salts, are convenient, stable, and readily available; however, multiple potential reduction pathways to generate active Pd(0) makes these systems more complex than they appear. Several other Pd(II) sources take advantage of the reactivity of allyl ligands to enable formation of metallated Pd(0) complexes in situ. For example, CpPd(allyl) and CpPd(cinnamyl) are known to react with phosphines and generate Pd(0) via reductive elimination, but this reactivity is ligand-specific.

A more common set of Pd(II) allyl sources are the chloride-bridged dimers. These palladium precursors are frequently used to generate active catalysts in situ, or are the basis of single-component precatalysts; however, comproportionation reactions can result in the formation of inactive Pd(I) μ-allyl dimers depending on the substitution pattern of the allyl ligand. To combat this, some in the art have designed a precatalyst scaffold (e.g., (η3-1-tBu-indenyl)₂(μ-Cl)₂Pd₂); however, one general drawback of allyl-based Pd(II) sources is the difficulty in coordinating very large ligands, including many biaryl phosphines that are highly active for C—N cross-coupling. Others in the art have demonstrated that chloride abstraction to generate cationic [LPd(allyl)]⁺ precatalysts is successful where L is large; however, this strategy is not suitable for in situ catalyst formation.

In stark contrast to the prevalence of Pd(II) sources for in situ catalyst formation, there is a paucity of Pd(0) sources available for the same purpose. Using a Pd(0) source has the advantage of not requiring a reduction step during catalyst formation/activation; however, Pd₂dba₃ and its various solvates are effectively the only option for a Pd(0) source in catalyst screening. While easily prepared and versatile, there are issues with Pd₂dba₃, including its relatively poor solubility (problematic from a solution-dosing standpoint in high-throughput screening), batch-to-batch inconsistency, differential reactivity based on solvate structure, and instability during storage either in solution or in the solid-state. There is also evidence that phosphine metallation rates vary by ligand structure.

Disclosed herein are embodiments of a new and versatile Pd(0) precursor complex and embodiments of a phosphorus-based Pd(0) catalyst derived therefrom. The disclosed Pd(0) precursor complex is particularly suited for HTE screening and offers several specific advantages for use in a variety of applications, including HTE, such as (but not limited to) good solubility, solution stability, and rapid/complete substitution of the Pd(0) precursor complex ligand with catalytically-relevant phosphorus-containing ligand groups to provide phosphorus-based Pd(0) catalysts. Embodiments of the disclosed Pd(0) precursor complex are superior at identifying ligand hits when evaluated alongside other common Pd sources. And, embodiments of the Pd(0) precursor complex and/or phosphorus-based Pd(0) catalyst are able to catalyze preparative-scale reactions (≥1 mmol) at low Pd loadings.

### III. Catalyst Embodiments

Disclosed herein are embodiments of a Pd(0) precursor complex and a phosphorus-based Pd(0) catalyst made using the complex. In particular embodiments, the Pd(0) precursor complex and/or phosphorus-based Pd(0) catalyst has a structure according to Formula I, illustrated below.

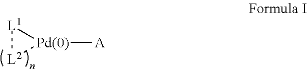

With reference to Formula I, the A group is a compound comprising at least one olefin moiety capable of coordinating with Pd(0); L¹ is a first ligand atom that is part of a first ligand group; L², if present (such as when n=1), is a second ligand atom that is part of a second ligand group; and n is 0 or 1. In particular embodiments, if L² is present, it is either indirectly bound to L¹ through one or more additional atoms to thereby provide a bidentate ligand group bound to the Pd(0); or it is not bound to L¹ and thereby provides a second ligand group, in addition to the first ligand group provided by L¹, bound to the Pd(0). In some other embodiments, n is 0 and thus L² is not present and thus L¹ is a first ligand atom that is part of a monodentate ligand group bound to the Pd(0).

In some embodiments, the A group is a cyclic or acyclic compound comprising at least one olefin moiety that coordinates with the Pd(0). In particular embodiments, the A group has a structure according to Formula II, illustrated below.

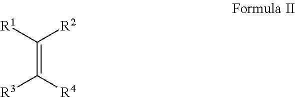

With reference to Formula II, each of R¹, R², R³, and R⁴ independently is (i) selected from hydrogen, aliphatic, aromatic, or an electron-withdrawing group provided that at least one of R¹, R², R³, or R⁴ is other than hydrogen; or (ii) R¹ and R³, or R² and R⁴ join together to provide a cyclic group, such as a 5- to 7-membered cyclic group, and the remaining R¹ and R³ groups, or R² and R⁴ groups are hydrogen. In particular embodiments, the A group is maleic anhydride (wherein, with reference to Formula II, R¹ and R³ are hydrogen and R² and R⁴ join together to provide the 5-membered anhydride). In other embodiments, the A group has a structure wherein at least one of R¹, R², R³, and R⁴ (e.g., wherein one, two, three, or four of R¹, R², R³, and R⁴) are an electron-withdrawing group, such as an ester group, a carboxyl group, a cyano group, an aldehyde group, a ketone group, or a nitro group, and any remaining R¹, R², R³, or R⁴ groups are hydrogen or phenyl. In some embodiments, the A group has a structure wherein R¹ is an ester or cyano, and each of R², R³, and R⁴ is hydrogen. In yet additional embodiments, the A group has a structure wherein one of R¹, R², R³, or R⁴ is an alkenyl group so as to provide a diene-containing compound comprising at least two olefin moieties that can be conjugated or unconjugated. Such diene-containing compounds can be cyclic or acyclic. In representative embodiments, the A group can be selected from any of the groups illustrated below.

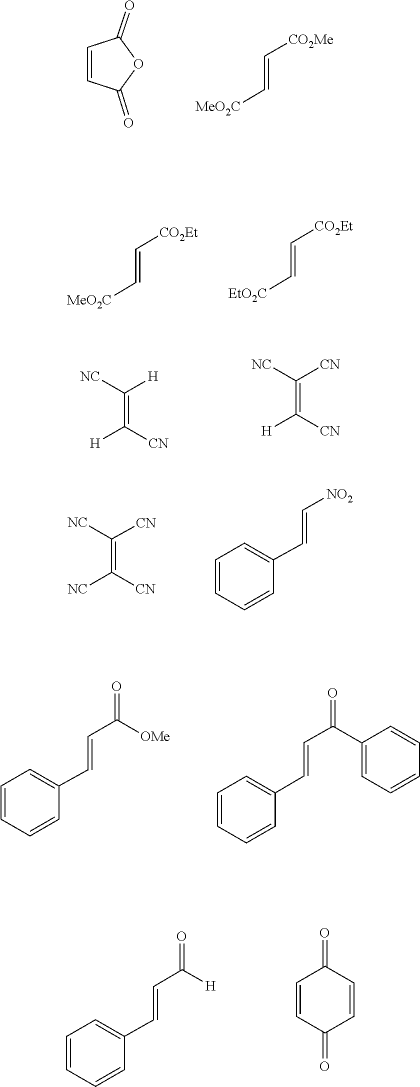

In some embodiments of Formula I, L¹ and L² are both nitrogen atoms that are part of a single bidentate ligand group. In some such embodiments, the two nitrogen atoms are indirectly bound to each other so as to provide a N,N-substituted diazabutadiene moiety. Such embodiments of Formula I can be used as precatalysts in methods described herein and can be used to generate phosphorus-based Pd(0) catalysts disclosed herein. Such embodiments of Formula I can have structure further satisfying Formula III, illustrated below. Also, when L¹ and L² are both nitrogen, each nitrogen atom is further bound to a B ring group or a B′ ring group as illustrated in Formula III.

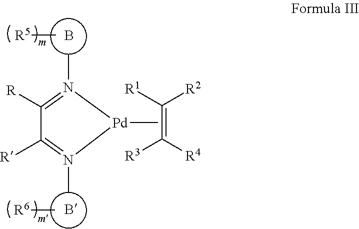

With reference to Formula III, each of R and R′ independently can be selected from hydrogen, aliphatic, heteroaliphatic, aromatic, or an organic functional group, or R and R′ can join together to provide a ring system, including an aromatic, heterocyclic, or alicyclic ring system; each B ring and B′ ring independently is an aromatic ring system; each R⁵ independently is selected from aliphatic, heteroaliphatic, haloaliphatic, aromatic, an organic functional group, or a combination thereof; each R⁶ independently is selected from aliphatic, heteroaliphatic, haloaliphatic, aromatic, an organic functional group, or a combination thereof; each and each of m and m′ independently is an integer selected from 0 to 10, such as 1 to 10, or 2 to 10, or 3 to 10, or 4 to 10, or 0, 1, 2, 3, 4, 5, 6, 7, 8, 9, or 10. In some embodiments, each of R and R′ independently can be selected from hydrogen, alkyl, alkenyl, alkynyl, heteroalkyl (e.g., alkoxy), heteroalkenyl, heteralkynyl, aryl, or heteroaryl; or join together to provide an aromatic or alicylic ring system. Each of R¹, R², R³, and R⁴ for the illustrated the A group can be as recited above for Formula II. In some such embodiments, each of R¹, R², R³, and R⁴ are selected as described herein to provide a maleic anhydride group. In other embodiments, each of R¹, R², R³, and R⁴ are selected as described herein to provide a fumarate group or a fumoranitrile group. In particular embodiments, each of the B and B′ ring groups is an aryl group, such as phenyl or naphthyl; each R⁵ and R⁶ independently is selected from alkyl, such as lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); haloalkyl, such as lower haloalkyl (e.g., CF₃); heteroalkyl, such as lower heteroalkyl (e.g., OMe); halide; cyano; ester; amide; or amine; and each of m and m′ independently is an integer selected from 0 to 5. In representative embodiments, each of the B and B′ rings is phenyl, each of m and m′ is 2 and each R⁵ and each R⁶ are each methyl or isopropyl. In some such embodiments, the two methyl or two isopropyl groups are bound to the phenyl ring at the ortho positions.

In some embodiments of Formula III, the N,N-substituted diazabutadiene moiety can have a structure wherein R and R′ join together to form a ring system. In some such embodiments, the ring system can comprise an aromatic ring system or an alicyclic group. In such embodiments, the fused ring system can be selected from phenanthrene, acenaphthene, camphor, and the like. Such complex embodiments can have a structure according to Formulas IIIA, IIIB, or IIIC.

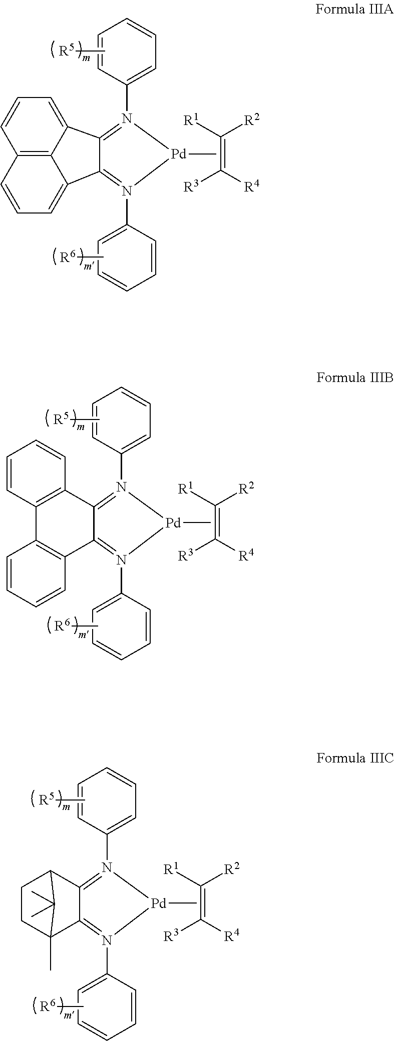

With reference to Formulas IIIA, IIIB, and IIIC, each of R⁵, R⁶, m, and m′ can be as recited above for Formula III and each of R¹, R², R³, and R⁴ for the illustrated the A group can be as recited above for Formula II and/or Formula III. In particular embodiments of Formulas IIIA, IIIB, and/or IIIC, each of m and m′ is 2 and each R⁵ and each R⁶ are either methyl or isopropyl.

In some embodiments, the N,N-substituted diazabutadiene moiety comprises R and R′ groups that are hydrogen. Such embodiments can have a structure according to Formula IIID, illustrated below. In particular embodiments of Formula IIID, each of m and m′ is 2 and each R⁵ and each R⁶ are either methyl or isopropyl.

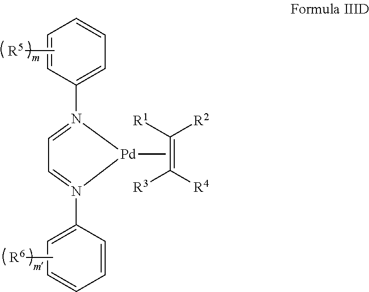

Representative, non-limiting Pd(0) precursor complexes of Formulas I, III, and/or lIlA-IIID are illustrated below.

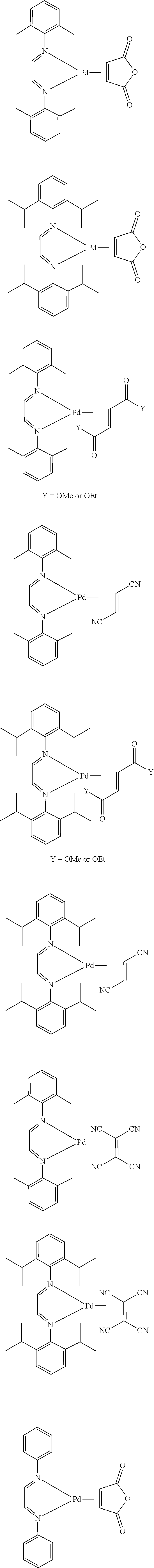

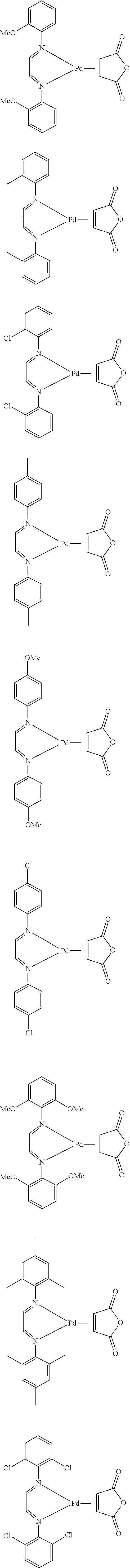

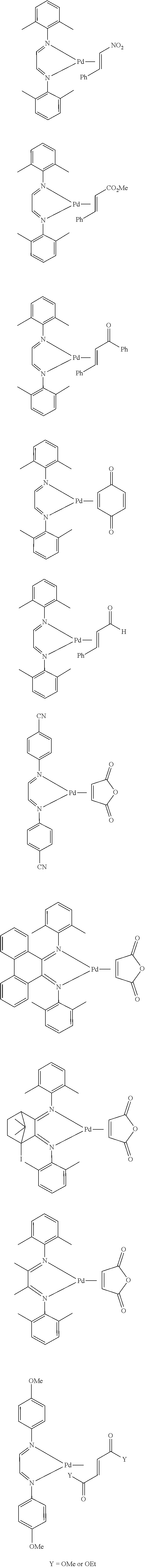

In an independent embodiment of a complex according to Formula IIID (or Formula III, where the B and B′ rings are phenyl), if (i) the A group (that is, the (R¹)(R²)C═C(R³)(R⁴) group) is dimethyl fumarate, diethyl fumarate, fumaronitrile, or tetracyanoethylene, (ii) m and m′ are both 1, and (iii) R⁵═R⁶, then R⁵ and R⁶ are not, or are other than, OMe. In yet another independent embodiment of a complex according to Formula IIIA (or Formula III, where the B and B′ rings are phenyl and the N,N-substituted diazabutadiene moiety further comprises a fused acenaphthene ring system), if (i) the A group is maleic anhydride, (ii) m and m′ are 1, and (iii) R⁵═R⁶, then R⁵ and R⁶ are not, or are other than, OMe, methyl, isopropyl, chloro, or trifluoromethyl. In yet another independent embodiment of a complex according to Formula IIIA (or Formula III, where the B and B′ rings are phenyl and the N,N-substituted diazabutadiene moiety further comprises a fused acenaphthene ring system), if (i) the A group is maleic anhydride, (ii) m and m′ are 2, and (iii) R⁵═R⁶, then R⁵ and R⁶ are not, or are other than, methyl, isopropyl, or trifluoromethyl. In an independent embodiment, the complex is not

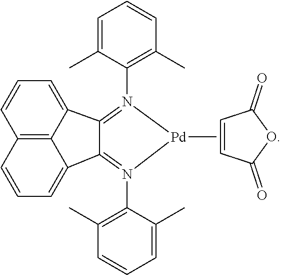

Pd(0) precursor complexes according to Formulas III and IIIA-IIID can be used as precatalysts to generate other Pd(0) complexes, such as phosphorus-based Pd(0) catalysts. In some such embodiments, phosphorus-based Pd(0) catalysts can have a structure according to Formula I, wherein n is 0 and thus L² is not present; and L¹ is a phosphorus atom. The phosphorus atom is further bound to three substituents (or one or more cyclic groups wherein two or three of the substituents are bound to one another) thereby providing a phosphorus-containing ligand group (or groups). In particular embodiments, L¹ is P(R⁷)(R⁸)(R⁹), as illustrated in Formula IV below.

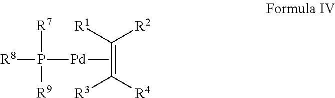

With reference to Formula IV, each of R¹, R², R³, and R⁴ are as recited for any of Formulas I-IIID and each of R⁷, R⁸, and R⁹ independently is selected from aliphatic, heteroaliphatic, haloaliphatic, haloheteroaliphatic, aromatic, or an organic functional group; or two or more of R⁷, R⁸, and R⁹ can join together, with the phosphorus atom to which they are attached, to provide a cyclic group (such as a heterocyclic ring system). In particular embodiments, each of R⁷, R⁸, and R⁹ independently is selected from alkyl, alkenyl, alkynyl, heteroalkyl, heteroalkenyl, heteroalkynyl, haloalkyl, haloalkenyl, haloalkynyl, haloheteroakyl, haloheteroalkenyl, haloheteroalkynyl, aryl, heteroaryl, or an organic functional group; or all of R⁷, R⁸, and R⁹ can join together, with the phosphorus atom to which they are attached, to provide a 1,3,5-triaza-7-phosphadamantyl group; or two of R⁷, R⁸, or R⁹ can be part of a single aromatic group (e.g., a binol group). In yet additional embodiments, each of R⁷, R⁸, and R⁹ independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); phenyl; pyridinyl; binaphthyl; alkoxy; aroxy; amine; 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; 2′,4′,6′-triisopropyl-2,3,4,5-tetramethyl-1,1′-biphenyl; 3,5-(CF₃)-phenyl; 4-OMe-phenyl; or 4-Cl-phenyl.

In an independent embodiment of Formula IV, if (i) the A group (that is, the (R¹)(R²)C═C(R³)(R⁴) group) is maleic anhydride, and (ii) two of R⁷, R⁸, and/or R⁹ are phenyl or cyclohexyl, then the remaining R⁷, R⁸, or R⁹ is not

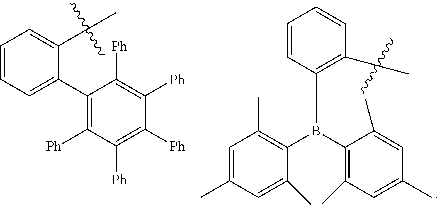

In another independent embodiment of Formula IV, if (i) the A group is maleic anhydride or a fumaronitrile, and (ii) two of R⁷, R⁸, and/or R⁹ are OEt, then the remaining R⁷, R⁸, or R⁹ is not OEt. In another independent embodiment of Formula IV, if (i) the A group is maleic anhydride, and (ii) two of R⁷, R⁸, or R⁹ form a binol group, then the remaining R⁷, R⁸, or R⁹ is not, or is other than, —OC(Ph)H—CH₂—SCH₂CH₃. In yet another independent embodiment of Formula IV, if (i) the A group is methyl fumarate, and (ii) two of R⁷, R⁸, and/or R⁹ are OEt or OPh, then the remaining R⁷, R⁸, or R⁹ is not, or is other than, OEt or OPh. In another independent embodiment of Formula IV, if (i) the A group is fumoranitrile, and (ii) two of R⁷, R⁸, or R⁹ form a binol group, the remaining R⁷, R⁸, or R⁹ is not, or is other than, —OC(Ph)H—CH₂—SCH₂CH₃.

In other embodiments, phosphorus-based Pd(0) catalysts can have a structure according to Formula I, wherein n is 1 and thus both L¹ and L² are present. In such embodiments, L¹ and L² are each phosphorus atoms that are bound to three substituents (or one or more cyclic groups wherein two or three of the substituents are bound to one another). In some embodiments, the L¹ and L² components are provided as two separate monodentate phosphorus-containing ligand groups. In other embodiments, the L¹ and L² components are provided as a bidentate ligand group wherein the L¹ and L² phosphorus atoms are indirectly bound to one another through one or more additional ligand group atoms (e.g., one or more carbon atoms and/or heteroatoms). In some embodiments, the Pd(0) precursor complex can have a structure according to Formula VA or VB, shown below. Formula VB shows embodiments comprising a bidentate ligand group wherein the L¹ and L² phosphorus atoms are indirectly bound to one another through substituents R⁹ and R¹⁰.

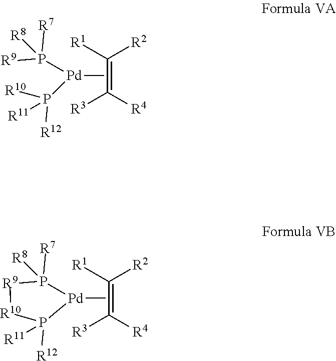

With reference to Formulas VA and VB, each of R¹, R², R³, and R⁴ are as recited for any of Formulas I-IV and each of R⁷, R⁸, R⁹, R¹⁰, R¹¹, and R¹² independently is selected from aliphatic, heteroaliphatic, haloaliphatic, haloheteroaliphatic, aromatic, or an organic functional group; or two or more of R⁷, R⁸, and R⁹ (and/or independently two or more of R¹⁰, R¹¹, and R¹²) can join together, with the phosphorus atom to which they are attached, to provide a cyclic group (such as a heterocyclic ring system); or, in the case of Formula VB, R⁹ and R¹⁰ independently are aliphatic, heteroaliphatic, haloaliphatic, haloheteroaliphatic, aromatic, or an organic functional group and are bound together through one or more carbon atoms and/or heteroatoms. In particular embodiments of Formula VA, each of R⁷, R⁸, R⁹, R¹⁰, R¹¹, and R¹² independently is selected from alkyl, alkenyl, alkynyl, heteroalkyl, heteroalkenyl, heteroalkynyl, haloalkyl, haloalkenyl, haloalkynyl, haloheteroakyl, haloheteroalkenyl, haloheteroalkynyl, aryl, heteroaryl, or an organic functional group; or all of R⁷, R⁸, and R⁹ (and/or all of R¹⁰, R¹¹, and R¹²) can join together, with the phosphorus atom to which they are attached, to provide a 1,3,5-triaza-7-phosphadamantyl group; or two of R⁷, R⁸, or R⁹ can be part of a single aromatic group (e.g., a binol group). In yet additional embodiments, each of R⁷, R⁸, R⁹, R¹⁰, R¹¹, and R¹² independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); phenyl; pyridinyl; binaphthyl; alkoxy, aroxy; amine; oxydibenzene; cyclopentadiene (including Fe-complexed cyclopentadiene ring systems, such as 1,1′-ferrocene); 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; 2′,4′,6′-triisopropyl-2,3,4,5-tetramethyl-1,1′-biphenyl; 3,5-(CF₃)-phenyl; 4-OMe-phenyl; or 4-Cl-phenyl.

In some embodiments of Formula VB, each of R⁷, R⁸, R¹¹, and R¹² independently is selected from lower alkyl (e.g., methyl; ethyl; propyl; isopropyl; butyl; iso-butyl; tert-butyl; sec-butyl; pentyl and any isomers or cyclic versions thereof; hexyl and any isomers or cyclic versions thereof; heptyl and any isomers or cyclic versions thereof; octyl and any isomers or cyclic versions thereof; nonyl and any isomers or cyclic versions thereof; decyl and any isomers or cyclic versions thereof); phenyl; pyridinyl; binaphthyl; alkoxy, aroxy; amine; oxydibenzene; cyclopentyldiene; 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; 2′,4′,6′-triisopropyl-2,3,4,5-tetramethyl-1,1′-biphenyl; 3,5-(CF₃)-phenyl; 4-OMe-phenyl; or 4-Cl-phenyl; and R⁹ and R¹⁰ are bound together to provide a xanthene ring system, a dimethyl-xanthene ring system, or a propyl group.

Representative catalysts of Formulas IV, VA, and VB are illustrated below.

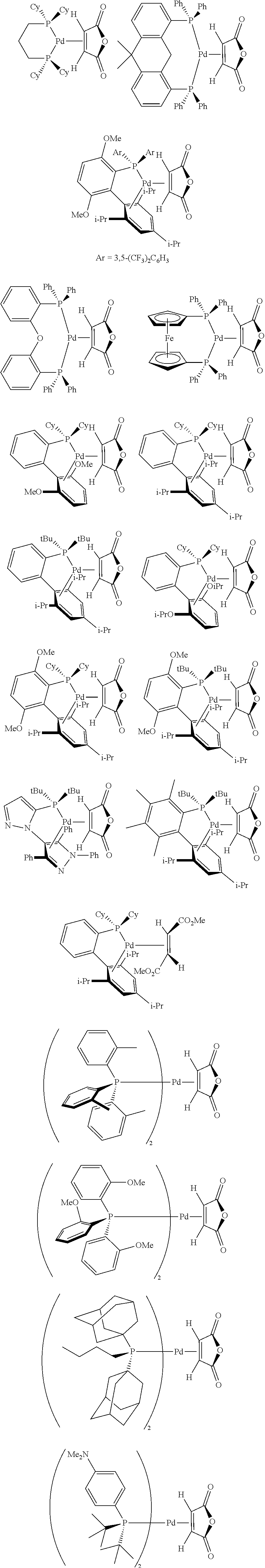

In an independent embodiment of Formula VA, if (i) the A group (that is, the (R¹)(R²)C═C(R³)(R⁴) group) is maleic anhydride, and (ii) two of R⁷, R⁸ and/or R⁹ and two of R¹⁰, R¹¹ and/or R¹² are phenyl, t-butyl, propylbenzene, 4-OMe-phenyl, 4-Cl-phenyl, or —SiMe₂CH₂CH₂(CF₂)₅—CF₃, then the remaining R⁷, R⁸, or R⁹ and remaining R¹⁰, R¹¹, or R¹² is not, or is other than, phenyl, propylbenzene, 2-pyrdinyl, 4-OMe-phenyl, 4-Cl-phenyl, or —SiMe₂CH₂CH₂(CF₂)₅—CF₃. In yet another independent embodiment of Formula VA, if (i) the A group is maleic anhydride and (iia) two of R⁷, R⁸ and/or R⁹ and two of R¹⁰, R¹¹ and/or R¹² are OMe or OPh, then the remaining R⁷, R⁸, or R⁹ and remaining R¹⁰, R¹¹, or R¹² is not, or is other than, OMe or OPh. In yet another independent embodiment of Formula VA, if (i) the A group is a fumarate, and (ii) two of R⁷, R⁸ and/or R⁹ and two of R¹⁰, R¹¹ and/or R¹² are phenyl, methyl, ethyl, or t-butyl, then the remaining R⁷, R⁸, or R⁹ and remaining R¹⁰, R¹¹, or R¹² is not, or is other than, methyl, ethyl, phenyl, or phenyl substituted with an imine; or (iib) if R⁷, R⁸, and R⁹, together with the phosphorus atom to which they are bound, form a 1,3,5-triaza-7-phosphadamantyl group, then R¹⁰, R¹¹, or R¹² do not also form a 1,3,5-triaza-7-phosphadamantyl group. In another independent embodiment of Formula VA, if (i) A group is a fumaronitrile and (iia) two of R⁷, R⁸ and/or R⁹ and two of R¹⁰, R¹¹ and/or R¹² are phenyl, then the remaining R⁷, R⁸, or R⁹ and remaining R¹⁰, R¹¹, or R¹² is not, or is other than, phenyl, binaphthyl, or 2-pyridinyl; or (iib) if R⁷, R⁸, and R⁹, together with the phosphorus atom to which they are bound, form a 1,3,5-triaza-7-phosphadamantyl group, then R¹⁰, R¹¹, or R¹² do not also form a 1,3,5-triaza-7-phosphadamantyl group.

In an independent embodiment of Formula VB, if (i) the A group (that is, the (R¹)(R²)C═C(R³)(R⁴) group) is maleic anhydride, maleate, or a fumarate, and if R⁷, R⁸, R¹¹, and R¹² are phenyl, then R⁹ and R¹⁰ do not form an ethyl group, a propyl group, or a 1,1′-ferrocene group. In another independent embodiment of Formula VB, if (i) the A group (that is, the (R¹)(R²)C═C(R³)(R⁴) group) is a fumaronitrile, and (ii) R⁷, R⁸, R¹¹, and R¹² are phenyl or cyclohexyl, then R⁹ and R¹⁰ do not form an ethyl group or a —CH₂N(Me)CH₂— group. In yet another independent embodiment of Formula VB, if (i) the A group is maleic anhydride and (ii) R⁷, R⁸, R¹¹, and R¹² are isopropyl, then R⁹ and R¹⁰ do not form a group having a structure:

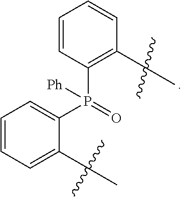 [SMILES: *c1ccccc1P(=O)(c1ccccc1)c1ccccc1*]

### IV. Method Embodiments

Also disclosed herein are methods for making and using the Pd(0) precursor complex and phosphorus-based Pd(0) catalyst embodiments of the present disclosure.

The Pd(0) precursor complex embodiments of the present disclosure can be made according to a method embodiment wherein a Pd₂dba₃ complex (e.g., Pd₂dba₃·CHCl₃) is used as a precursor. In some embodiments, the method comprises exposing the Pd₂dba₃, in the presence of a solvent, to a suitable ligand compound, which provides the L² and/or L¹ components of Formula I and an A group, to provide a reaction mixture. In particular embodiments, the solvent is acetone. In particular embodiments, the L² and L¹ components of Formula I can be provided by a single N,N-substituted diazabutadiene ligand compound to provide complexes wherein L²=L¹=N atoms of the N,N-substituted diazabutadiene group.

In some embodiments, the method can be conducted at ambient temperature and the reaction mixture can be allowed to mix for a suitable period of time to produce the desired Pd(0) precursor complex. The resulting Pd(0) precursor complex can be isolated using a suitable solid compound isolation technique, such as a precipitation method. In particular embodiments, a precipitation method is used whereby the reaction mixture is exposed to an ether solvent (e.g., tert-butyl methyl ether) and stirred in this solvent for a suitable period of time (e.g., 15 minutes). The resulting slurry can be decanted to isolate a solid therefrom, which can then be dissolved in acetone and filtered to remove any palladium black. This washing procedure can be repeated any number of times. The combined filtrates from the one or more washing steps can then be concentrated to provide the isolated complex as a solid.

In some additional embodiments, the method can further comprise making the Pd₂dba₃ compound from Pd(OAc)₂ by exposing Pd(OAc)₂ to dibenzylideneacetone (or “dba”) in the presence of a base and solvent (e.g., an alcohol solvent, such as methanol).

Method embodiments according to the above disclosure are summarized in Scheme 1, below. A representative method embodiment with representative L¹ and L² components wherein L¹=L²=N atoms of a N,N-substituted diazabutadiene group are illustrated in Scheme 2.

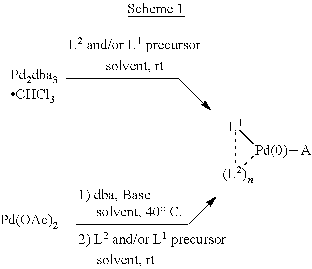

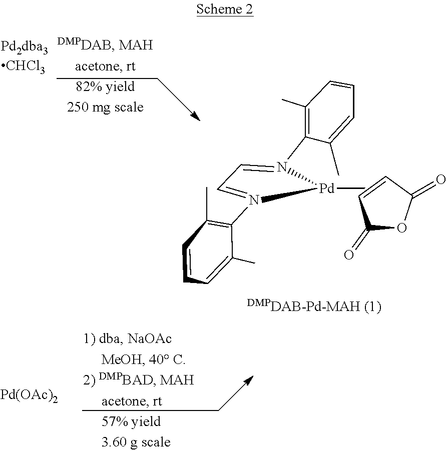

In yet additional embodiments, the method can further comprise converting the Pd(0) precursor complex to other Pd(0) precursor complex embodiments, such as phosphorus-based Pd(0) catalysts embodiments with L² and/or L¹ components that provide phosphorus-containing ligand groups, such as in catalyst embodiments of Formulas IV, VA, and VB described herein. Such method embodiments can comprise exposing a Pd(0) precursor complex having a structure according to any of Formulas III and/or IIIA-IIID to a mono- or bidentate phosphorus-containing ligand group, such as a mono- or bidentate phosphine compound, a phosphite compound, or a phosphoramidite compound, in the presence of a solvent (e.g., THF) and allowing the resulting reaction mixture to mix for a suitable time period. The method can further comprise isolating the phosphorus-based Pd(0) catalyst by removing the THE solvent and triturating the resulting solid with a different solvent (e.g., hexanes or diethyl ether) and then decanting the liquid phase. This can be repeated one or more times until any excess reactants are removed. The desired product can be isolated as a solid after removing any remaining solvent. Such method embodiments provide rapid conversion to the corresponding phosphorus-based Pd(0) catalyst, even at ambient temperature, with minimal to no side-product formation. In some embodiments, this transformation of a N,N-substituted diazabutadiene precatalyst (e.g., complexes according to Formulas III, IIIA, IIIB, IIIC, or IIID) can be carried out in situ when the N,N-substituted diazabutadiene precatalyst is used in a Pd-mediated coupling reaction, such as those described herein.

The disclosed Pd(0) precursor complex embodiments and/or phosphorus-based Pd(0) catalysts can be used in multiple different applications. For example, the Pd(0) precursor complex embodiments can be used for Pd-catalyzed chemical reactions, including those involved in HTE. In particular embodiments, Pd(0) precursor complexes according to any one of Formulas III or IIIA-IIID can be used as precatalysts in myriad different Pd-catalyzed chemical reactions. Exemplary such methods are described below and are provided solely as non-limiting examples.

The Pd(0) precursor complex embodiments disclosed herein can be used in a method wherein a first coupling partner and a second coupling partner are bound together. The method can comprise exposing the first coupling partner and/or the second coupling partner to a Pd(0) precursor complex according to Formula III (or any one of Formula IIIA-IIID) and a phosphorus-containing ligand group to promote forming a chemical bond between the first coupling partner and the second coupling partner. In such embodiments, the method can further comprising providing a solvent and/or a base. In particular embodiments, the method can further comprise first combining the Pd(0) precursor complex according to Formula III (or any one of Formula IIIA-IIID) and the phosphorus-containing ligand group to generate a phosphorus-based Pd(0) catalyst according to Formulas IV or VA or VB and then exposing the phosphorus-based Pd(0) catalyst to the first coupling partner and second coupling partner; however, the components of the method can be added in any order. In some embodiments, the first coupling partner and the second coupling partner can be combined before adding the Pd(0) precursor complex and the phosphorus-containing ligand group. In other embodiments, the first coupling partner can first be combined with the Pd(0) precursor complex and the phosphorus-containing ligand group, followed by adding the second coupling partner. In yet other embodiments, the second coupling partner can first be combined with the Pd(0) precursor complex and the phosphorus-containing ligand group, followed by adding the first coupling partner. In additional embodiments, the first and/or second coupling partners can first be exposed to the Pd(0) precursor complex, followed by adding the phosphorus-containing ligand group. In other additional embodiments, the first and/or second coupling partners can first be exposed to the phosphorus-containing ligand group, followed by adding the Pd(0) precursor complex. The amount of the Pd(0) precursor complex can range from 0.001 mol % to 40 mol %, such as 0.01 mol % to 15 mol %, or 0.1 mol % to 5 mol %. In some embodiments, the phosphorus-containing ligand group can be added in an amount so as to provide 1 to 2 (e.g., 1, 1.2, 1.5, or 1.75) mol equivalents relative to the Pd(0) precursor complex.

In some embodiments, the Pd(0) precursor complex can be used to promote different types of Pd-mediated couplings. Buchwald-Hartwig amination is one example of a Pd-mediated coupling. This type of coupling is one of the most prevalent palladium-catalyzed cross couplings in the art. Solely by way of example, and as described by particular working embodiments of the present disclosure, primary amines and second amines can be coupled to aryl halides using Pd(0) precursor complex embodiments disclosed herein. By way of example, benzylamine (a primary amine) can be coupled with 2-chloropyridine using a Pd(0) precursor complex as a precatalyst and further including a phosphorus-containing ligand group to thereby generate, in situ, a phosphorus-based Pd(0) catalyst. In one such embodiment, benzylamine and 2-chloropyridine were reacted with the Pd(0) precursor complex ^{DMP}DAB-Pd-MAH in THE at 60° C. for 1 hour with NaO-tBu and 26 mol % of a phosphorus-containing ligand group (e.g., a phosphine) and 13 mol % of Pd loading. With reference to this exemplary method, normalized fractions between HPLC product peak area and internal standard peak area (see Table 2 of Example 9A) indicated that the ^{DMP}DAB-Pd-MAH exhibited superior catalytic activity in this C—N coupling, compared to comparative Pd sources currently used in the art (i.e., Pd(OAc)₂, [Pd(allyl)Cl]2, and Pd₂dba₃·CHCl₃). In yet another example, a secondary amine (e.g., morpholine) was coupled with 1-bromo-4-methoxybenzene under the same reaction conditions, but at a temperature of 80° C. With reference to this exemplary method, various phosphorus-based Pd(0) catalyst embodiments generated in situ from the ^{DMP}DAB-Pd-MAH Pd(0) precatalyst (e.g., BrettPhos/^{DMP}DAB-Pd-MAH and RuPhos/^{DMP}DAB-Pd-MAH) exhibited good catalytic activity. Preparative scale for these two C—N couplings with much lower loading of isolated pre-ligated precatalysts or catalyst generated in situ from ^{DMP}DAB-Pd-MAH and a corresponding phosphorus-containing ligand group (or ligand groups) also gave excellent product yields (see Table 3 of Example 9B).

Other reactions can be carried out using Pd(0) precursor complex embodiments disclosed herein, such as the amination of sulfonamides, which can provide a N-arylsulfonamide substructure that is commonly found in marketed medicinal drugs. In exemplary embodiments of such a reaction, a Pd(0) precursor complex having a structure according to Formula III (e.g., such as one of Formulas IIIA-IIID) can be used in combination with a phosphine ligand to generate a phosphorus-based Pd(0) catalyst (e.g., such as a catalyst according to Formula IV, VA, or VB), in situ, that can catalyze such couplings at higher conversions than comparative Pd sources, such as Pd(OAc)₂, [Pd(allyl)Cl], and Pd₂dba₃·CHCl₃. In a representative embodiment, ^{DMP}DAB-Pd-MAH was used in combination with phosphine ligands selected from Xantphos, Xphos, tBuXPhos, Me₄tBuXPhos, Bippyphos, and/or JackiePhos. Results for these representative examples are provided by Table 4 of Example 9C.

In yet other embodiments, the Pd(0) precursor complex can be used to carryout challenging and N1-selective C—N coupling of unsymmetric imidazoles. Exemplary embodiments of such reactions are described herein in working examples (such as in Example 9D). In a representative embodiment, ^{DMP}DAB-Pd-MAH was used in combination with phosphine ligands selected from BrettPhos, Xphos, tBuXPhos, tBuBrettPhos, Me₄tBuXPhos, and/or Bippyphos. Results from these representative examples are provided by Table 5 of Example 9D.

In addition to C—N couplings, the Heck reaction, another prevalent Pd-catalyzed coupling with excellent functional group tolerance, can be carried out using Pd(0) precursor complexes disclosed herein. This type of arylation is extensively applied in organic synthesis, particularly in total synthesis of natural products. In some embodiments of such methods, a Pd(0) precursor complex according to Formula III and/or IIIA-IIID can be used in combination with a phosphorus-containing ligand group to provide a phosphorus-based Pd(0) catalyst according to Formula IV, VA, or VB that can catalyze the Heck reaction between two coupling partners. In exemplary embodiments of this method, ^{DMP}DAB-Pd-MAH gave higher yields than comparative Pd sources, such as Pd(OAc)₂, [Pd(allyl)Cl]2, and Pd₂dba₃·CHCl₃, even on scale-up. Phosphine ligand compounds selected from PCy₃, XPhos, P(o-tol)₃, cataCXium A, dppp, and P(tBu)₃ were used. Results from these representative examples are provided by Table 6A of Example 9E.

In yet another embodiment, C—O coupling can be carried out using Pd(0) precursor complex embodiments disclosed herein. This type of coupling is attractive due to the common presence of C—O bonds in a variety of interesting compounds, ranging from biological products to polymers to pharmaceuticals. Exemplary embodiments of such reactions are described herein in working examples (e.g., see Example 9F). In some embodiments, a Pd(0) precursor complex according to Formula III and/or IIIA-IIID was used in combination with a phosphorus-containing ligand group to provide a phosphorus-based Pd(0) catalyst according to Formula IV, VA, or VB. In representative examples, ^{DMP}DAB-Pd-MAH was used in combination with BrettPhos, XPhos, tBuXPhos, tBuBrettPhos, Me4tBuXPhos, or Bippyphos. Results from these representative examples are provided in Table 7 of Example 9F.

As another example that demonstrates the applicability of catalyst precursor embodiments disclosed herein, a screen for the synthesis of spiro-OMeTAD, an important hole-transport material perovskite-based solar cells, is described herein. Exemplary embodiments of such reactions are described herein in working examples (e.g., see Example 9G). In some embodiments, a Pd(0) precursor complex according to Formula III and/or IIIA-IIID was used in combination with a phosphorus-containing ligand group (e.g., BrettPhos, XPhos, tBuXPhos, SPhos, RuPhos, or XantPhos) to provide a phosphorus-based Pd(0) catalyst to Formula IV, VA, or VB. In particular embodiments using ^{DMP}DAB-Pd-MAH as a precatalyst, the Pd(0) precursor complex embodiments generated from the ^{DMP}DAB-Pd-MAH and XPhos, SPhos, RuPhos, and XantPhos ligands exhibited good catalytic activity. Results from these representative examples are provided in Table 8 of Example 9G.

In addition to C—N couplings, Mizoroki-Heck couplings (e.g., arylation of methyl methacrylate by p-bromoacetophenone) can be carried out using Pd(0) precursor complex embodiments disclosed herein. Results from an exemplary embodiment are provided in Table 9 of Example 9H.

### V. Overview of Several Embodiments

Disclosed herein are embodiments of a complex, having a structure according to Formula III (as disclosed herein), wherein each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; each of R and R′ independently is selected from hydrogen, aliphatic, heteroaliphatic, aromatic, or an organic functional group, or R and R′ together to provide an aromatic, heterocyclic, or alicyclic ring system; each of the B ring and B′ ring independently is an aromatic ring system; each R⁵ independently is selected from aliphatic, heteroaliphatic, haloaliphatic, aromatic, an organic functional group, or a combination thereof; each R⁶ independently is selected from aliphatic, heteroaliphatic, haloaliphatic, aromatic, an organic functional group, or a combination thereof; and each of m and m′ independently is an integer selected from 0 to 10.

In some embodiments, R¹ and R³, or R² and R⁴ join together to provide a 5-membered cyclic group.

In any or all of the above embodiments, the 5-membered cyclic group is maleic anhydride.

In any or all of the above embodiments, the B ring and B′ ring are both phenyl.

In any or all of the above embodiments, each R⁵ is lower alkyl; lower haloalkyl; lower heteroalkyl; halide; cyano; ester; amide; or amine.

In any or all of the above embodiments, each R⁶ is lower alkyl; lower haloalkyl; lower heteroalkyl; halide; cyano; ester; amide; or amine.

In any or all of the above embodiments, the lower alkyl group is methyl or isopropyl.

In any or all of the above embodiments, each of m and m′ independently is 2.

In any or all of the above embodiments, R and R′ are hydrogen or join together to provide an aromatic or alicyclic ring system.

In any or all of the above embodiments, the complex has a structure according to Formula IIIA, IIIB, IIIC, or IIID as disclosed herein.

In any or all of the above embodiments, the complex has a structure according to any complex species disclosed herein.

Also disclosed herein are embodiments of a catalyst having a structure according to Formula IV, VA, or VB (as disclosed herein), wherein for Formulas IV or VA, each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; each of R⁷, R⁸, R⁹, R¹⁰, R¹¹, and R¹² independently is selected from aliphatic, heteroaliphatic, haloaliphatic, haloheteroaliphatic, aromatic, or an organic functional group; or two or more of R⁷, R⁸, and R⁹, and/or independently two or more of R¹⁰, R¹¹, and R¹², can join together, with the phosphorus atom to which they are attached, to provide a heterocyclic ring system; and wherein for formula VB, each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; or each of R⁷, R⁸, R¹¹, and R¹² independently is selected from aliphatic, heteroaliphatic, haloaliphatic, haloheteroaliphatic, aromatic, or an organic functional group and R⁹ and R¹⁰ independently are aliphatic, heteroaliphatic, haloaliphatic, haloheteroaliphatic, aromatic, or an organic functional group and are bound together through one or more carbon atoms and/or heteroatoms.

In any or all of the above embodiments, R¹ and R³, or R² and R⁴, join together to provide a 5-membered cyclic group.

In any or all of the above embodiments, the 5-membered cyclic group is maleic anhydride.

In any or all of the above embodiments, the catalyst has a structure according to Formula IV and each of R⁷ and R⁸ is phenyl, cyclohexyl, t-butyl, or 3,5-(CF³)₂-phenyl, and R⁹ is selected from 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; or 2′,4′,6′-triisopropyl-2,3,4,5-tetramethyl-1,1′-biphenyl.

In any or all of the above embodiments, the catalyst has a structure according to Formula VA and each of R⁷, R⁸, R¹¹, and R¹² independently is phenyl or cyclohexyl and R⁹ is selected from 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; or 2′,4′,6′-triisopropyl-2,3,4,5-tetramethyl-1,1′-biphenyl.

In any or all of the above embodiments, the catalyst has a structure according to Formula VA and each of R⁷, R⁸, R⁹, R¹⁰, R¹¹, and R¹² independently is aromatic or aliphatic.

In any or all of the above embodiments, the catalyst is selected from any of the catalyst species disclosed herein

Also disclosed herein are embodiments of a method of making the complex according to any or all of the above complex embodiments, comprising exposing a Pd₂dba₃ complex to a ligand compound comprising a N,N-substituted diazabutadiene group to form a reaction mixture.

In any or all of the above embodiments, the ligand group comprising the N,N-substituted diazabutadiene group is N,N′-bis(2,6-dimethylphenyl)ethan-1,2-diimine.

In any or all of the above embodiments, the method further comprises isolating a complex according to any or all of the above embodiments by exposing the reaction mixture to tert-butyl methyl ether, stirring the reaction mixture with the tert-butyl methyl ether to form a slurry; decanting the slurry to isolate a solid therefrom, and optionally filtering the solid to remove any palladium black.

Also disclosed herein are embodiments of a method of making the catalyst according to any or all of the above catalyst embodiments, comprising exposing a phosphorus-containing ligand group to a complex having a structure according to Formula III, wherein each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; each of R and R′ independently is selected from hydrogen, aliphatic, heteroaliphatic, aromatic, or an organic functional group, or R and R′ together to provide an aromatic, heterocyclic, or alicyclic ring system; each of the B ring and B′ ring independently is an aromatic ring system; each R⁵ independently is selected from aliphatic, heteroaliphatic, haloaliphatic, aromatic, an organic functional group, or a combination thereof; and each R⁶ independently is selected from aliphatic, heteroaliphatic, haloaliphatic, aromatic, an organic functional group, or a combination thereof; and each of m and m′ independently is an integer selected from 0 to 10.

In any or all of the above embodiments, the phosphorus-containing ligand group is selected from 1,3-bis(diphenylphosphino)propane, Bis[(2-diphenylphosphino)phenyl] ether, 4,5-Bis(diphenylphosphino)-9,9-dimethylxanthene, 1,1′-Bis(diphenylphosphino)ferrocene, 2-Dicyclohexylphosphino-2′,6′-dimethoxybiphenyl, 2-Dicyclohexylphosphino-2′,6′-diisopropoxybiphenyl, 2-Dicyclohexylphosphino-2′,4′,6′-triisopropylbiphenyl, 2-Di-tert-butylphosphino-2′,4′,6′-triisopropylbiphenyl, 2-(Dicyclohexylphosphino)3,6-dimethoxy-2′,4′,6′-triisopropyl-1,1′-biphenyl, 2-(Di-tert-butylphosphino)-2′,4′,6′-triisopropyl-3,6-dimethoxy-1,1′-biphenyl, 5-(Di-tert-butylphosphino)-1′, 3′, 5′-triphenyl-1′H-[1,4′]bipyrazole, 2-Di-tert-butylphosphino-3,4,5,6-tetramethyl-2′,4′,6′-triisopropyl-1,1′-biphenyl, 2-{Bis[3,5-bis(trifluoromethyl)phenyl]phosphino}-3,6-dimethoxy-2′,4′,6′-triisopropyl-1,1′-biphenyl, or Di(1-adamantyl)-n-butylphosphine.

Also disclosed herein are embodiments of a method, comprising using a complex or catalyst according to any or all of the above complex and/or catalyst embodiments as a catalyst in a palladium-mediated coupling reaction.

### VI. EXAMPLES

Unless otherwise stated, all manipulation and procedures were conducted within a glovebox under a dry, oxygen-free N₂ atmosphere. The diimine ligands as well as (dba)₃Pd₂·CHCl₃ were synthesized in-house following established procedures. MAH was commercially purchased and used as received. Unless otherwise stated, reaction and wash solvents were either purchased as anhydrous grade and used as received or taken through multiple freeze-pump-thaw cycles and stored over 4 Å molecular sieves. All phosphine ligands employed were commercially purchased and used as received. All NMR solvents were commercially purchased and used as received. NMR spectra were acquired on Bruker 300 MHz or 500 MHz NMR spectrometers. IR spectra were obtained using a Thermo Scientific IR200 Infrared spectrometer, using anhydrous KBr and pellet forming apparatuses which were stored in a desiccator.

### Example 1

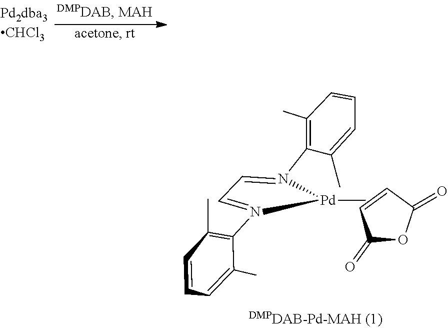

From Pd₂dba₃·CHCl₃: This synthesis was conducted under ambient atmosphere using commercial materials as-received. A 250 mL round bottom flask containing a stirbar was charged with Pd₂dba₃·CHCl₃ (251.5 mg, 0.243 mmol), N,N′-bis(2,6-dimethylphenyl)ethan-1,2-diimine (134.4 mg, 0.510 mmol, 2.1 equivalents), and maleic anhydride (51.4 mg, 0.522 mmol, 2.15 equivalents). Acetone (45 mL) was added to dissolve/suspend the components. The reaction mixture was stirred at room temperature for three hours, producing a dark purple/red homogeneous solution. The reaction solution was concentrated under vacuum to approximately 2 mL. TBME (40 mL) was added and the solution was stirred for 15 minutes, producing a purple/red slurry. After the solid settled, the TBME was decanted, leaving a purple/red solid. The solid was dissolved in a minimum of acetone and filtered through Celite to remove palladium black. The Celite bed was thoroughly rinsed with acetone until the rinsings were colorless, and the combined filtrate was evaporated under vacuum. The resulting purple/red solid was washed with TBME (3×10 mL) and dried under vacuum to give complex 1 (187 mg, 82% yield).

### Example 2

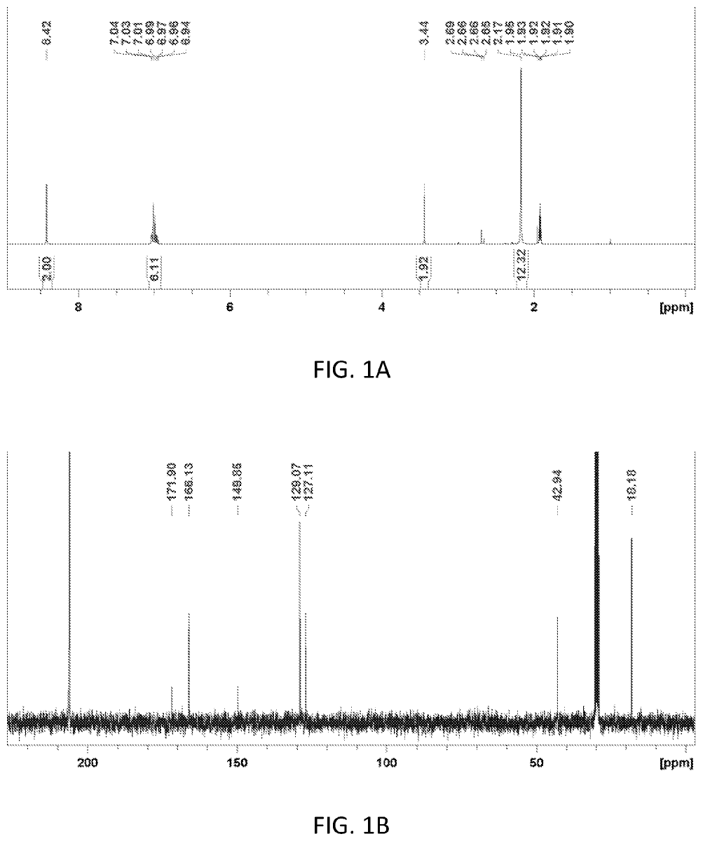

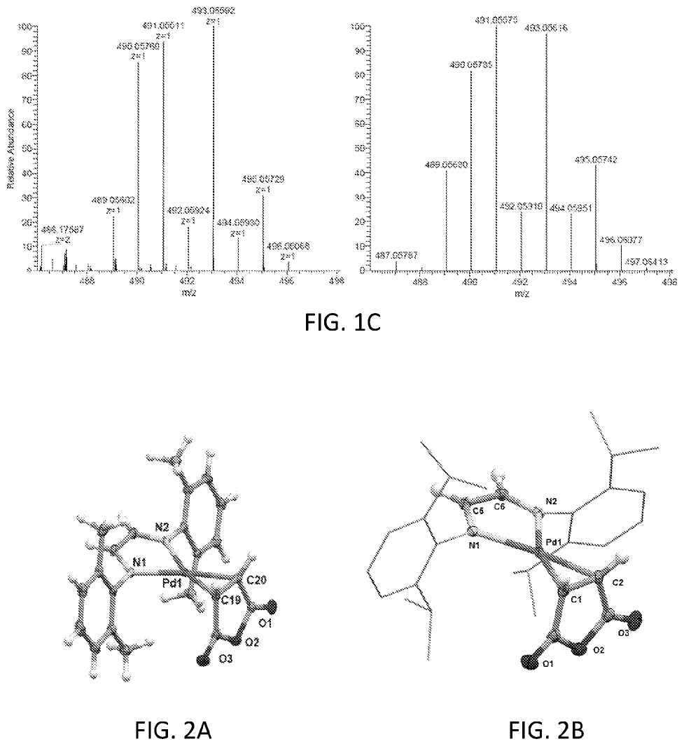

From Pd(OAc)₂: A 1 L round bottom flask containing a stirbar was charged with dibenzylideneacetone (6.26 g, 26.7 mmol, 2 equivalents) and sodium acetate (10.96 g, 133.6 mmol, 10 equivalents). Methanol (250 mL) was added and the mixture stirred to ensure dissolution of the dba. With stirring, solid Pd(OAc)₂ (3.00 g, 13.4 mmol) was added through a powder funnel, which was rinsed with methanol (50 mL) to ensure quantitative transfer. The flask was immersed in an oil bath kept at 40-45° C. The reaction mixture was stirred vigorously at this temperature for 3 hours. The flask was then cooled to room temperature. The resulting dark slurry was filtered through filter paper in a Buchner funnel to collect the crude “Pd(dba)₂” solid. The solid was washed successively with methanol (3×30 mL), water (3×30 mL), and acetone (2×10 mL). This solid was then transferred to a 1 L round bottom flask containing a stir bar, and slurried in acetone (300 mL). Solid N,N′-bis(2,6-dimethylphenyl)ethan-1,2-diimine (3.71 g, 14.0 mmol, 1.05 equivalents) and maleic anhydride (1.38 g, 14.0 mmol, 1.05 equivalents) were added through a powder funnel, which was rinsed with acetone (100 mL) to ensure quantitative transfer. The reaction mixture was stirred at room temperature for 3 hours. The dark red solution was filtered through a bed of Celite using a medium porosity frit to remove palladium black. The Celite bed was thoroughly rinsed with acetone until the rinsings were colorless, and the combined filtrate was evaporated under vacuum. TBME (100 mL) was added and the solid triturated in the flask. The purple/red solid was collected by suction filtration, and the filter cake was washed with TBME (6×10 mL, until rinsings are colorless) to completely remove dba. The solid was dried under vacuum to give complex 1 (3.60 g, 57% yield from Pd(OAc)₂). ¹H NMR: (300 MHz; acetone-d₆) δ 2.17 (s, 12H, 4×Ar-CH₃), 3.44 (s, 2H, —CH═CH—), 6.99 (m, 6H, 6×Ar-H), 8.42 (s, 2H, 2×—CH═NAr). ¹³C{¹H} NMR: (75 MHz; acetone-d₆) 18.2 (4×Ar—CH₃), 42.9 (—CH═CH—), 127.1 (Ar), 129.1 (Ar), 149.9 (Ar), 166.1 (—N═C—C═N—), 171.9 (2×C═O). HRMS (ESI) of [C₂₂H₂₂N₂O₃Pd·Na]⁺ (major isotopomer, sodium adduct): 491.05575 (calc'd); 491.05511. See FIGS. 1A-1C for spectra.

### Example 3

Complex 1 is a soluble, air and moisture stable mononuclear Pd(0) complex. Analysis by NMR spectroscopy generates simple and easy to interpret spectra that are consistent with the given structure, providing an excellent method to quickly assess purity. This is in contrast to the solution behavior of Pd₂dba₃, where the presence of multiple species and extensive magnetic inequivalence leads to complex NMR spectra, and poor solubility leads to low signal-to-noise. The solid-state molecular structure of complex 1 also was obtained via single-crystal X-ray diffraction (see FIG. 2A), confirming the proposed structure.

Single crystals of C₂₂H₂₂N₂O₃Pd (complex 1) were selected using a MitEGen loop using paratone oil. A suitable crystal was selected and run Bruker APEX-II CCD diffractometer. The crystal was kept at 99.96 K during data collection. Using Olex2, the structure was solved with the olex2.solve structure solution program using Charge Flipping and refined with the XL refinement package using Least Squares minimization.

Crystal Data for C₂₂H₂₂N₂O₃Pd (M=468.81 g/mol): triclinic, space group P-1 (no. 2), a=9.1137(9) Å, b=10.2220(10) Å, c=12.2985(12) Å, α=70.989(2)°, β=70.567(2)°, γ=71.163(2)°, V=991.53(17) Å³, Z=2, T=99.96 K, μ(MoKα)=0.961 mm⁻¹, Dcalc=1.570 g/cm³, 16832 reflections measured (3.622°≤2Θ≤56.148°), 4746 unique (Rᵢₙₜ=0.0136, R_{sigma}=0.0130) which were used in all calculations. The final R₁ was 0.0162 (I>2σ(I)) and wR₂ was 0.0427 (all data).

### Example 4

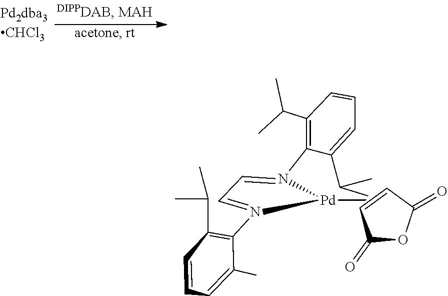

This example describes the synthesis of bis(N,N′-Bis(2,6-diisopropylphenyl)ethan-1,2-diimine)(η₂-furan-2,5-dione)palladium(0). This synthesis was conducted in a fume hood in open air using non-purified solvents. A 250 mL round bottom flask was charged with 103.mg of (dba)₃Pd₂·CHCl₃, 78.1 mg of N,N′-Bis(2,6-diisopropylphenyl)ethan-1,2-diimine, 20.5 mg of MAH, 25 mL of acetone and a magnetic stir bar. The reaction mixture was stirred at room temperature for 3 hours which produced a dark red homogenous solution. The reaction solution was concentrated using a rotovap to approximately 2 mL, and 25 mL of TBME was then added. This solution was passed through a ground glass frit with a Celite bed to remove any deposited palladium black and the filtrate was collected in a 30 mL glass vial. The vial was then placed upon a rotovap and a red solid was isolated. The solid was dissolved in a minimum amount of dichloromethane and 3 volume equivalents of pentane was carefully layered on top. The vial was then carefully placed in a freezer. After a week, the vial was retrieved, the bulk of the mother liquor was carefully removed using a Pasteur pipette and stored in a 30 mL glass vial in a freezer, leaving behind the dba byproduct. After three weeks, the mother liquor was retrieved from the freezer and large crystals of the desired complex were observed and isolated. Yield not determined. ¹H NMR: (300 MHz; CDCl₃) δ 1.12 (two overlapping d, 12H, 4×—CH₃), 1.29 (d, 12H, J=7 Hz, 4×—CH₃), 3.14 (br m, 4H, 4×—CH—), 3.63 (s, 2H, —CH═CH—), 7.19 (m, 6H, 6×Ar—H), 8.10 (s, 2H, 2×—CH═NAr).

Single crystals of C₃₁H₄₀Cl₂N₂O₃Pd (dichloromethane solvate) were selected using a MitEGen loop using paratone oil. A suitable crystal was selected and mounted on a ‘Bruker APEX-II CCD’ diffractometer. The crystal was kept at 99.98 K during data collection. Using Olex2, the structure was solved with the olex2.solve structure solution program using Charge Flipping and refined with the XL refinement package using Least Squares minimisation.

Crystal Data for C₃₁H₄₀Cl₂N₂O₃Pd (M=665.95 g/mol): monoclinic, space group P2₁/c (no. 14), a=10.3444(11) Å, b=16.2472(17) Å, c=19.627(2) Å, β=103.898(2)°, V=3202.1(6) Å3, Z=4, T=99.98 K, μ(MoKα)=0.779 mm-1, Dcalc=1.381 g/cm3, 55978 reflections measured (3.294°≤2Θ≤5 56.066°), 7712 unique (Rᵢₙₜ=0.0290, R_{sigma}=0.0194) which were used in all calculations. The final R₁ was 0.0215 (I>2σ(I)) and wR₂ was 0.0526 (all data). See FIG. 2B.

### Example 5

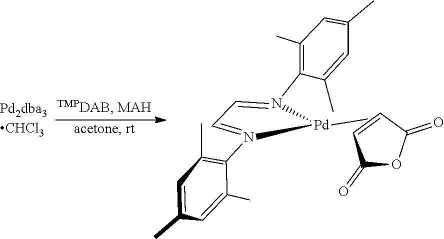

This synthesis was conducted under ambient atmosphere using commercial materials as-received. A 250 mL round bottom flask containing a stirbar was charged with Pd₂dba₃·CHCl₃ (250.0 mg, 0.242 mmol), N,N′-bis(2,4,6-trimethylphenyl)ethan-1,2-diimine (148.3 mg, 0.510 mmol, 2.1 equivalents), and maleic anhydride (49.7 mg, 0.510 mmol, 2.1 equivalents). Acetone (45 mL) was added to dissolve/suspend the components. The reaction mixture was stirred at room temperature for three hours, producing a dark purple/red homogeneous solution. The reaction solution was concentrated under vacuum to approximately 2 mL. TBME (40 mL) was added and the solution was stirred for 15 minutes, producing a purple/red slurry. After the solid settled, the TBME was decanted, leaving a purple/red solid. The solid was dissolved in a minimum of acetone and filtered through Celite to remove palladium black. The Celite bed was thoroughly rinsed with acetone until the rinses were colorless, and the combined filtrate was evaporated under vacuum. The resulting purple/red solid was washed with TBME (7×10 mL) and dried under vacuum to give the desired complex (105 mg, 44% yield). ¹H NMR: (300 MHz; acetone-d₆) δ 2.26 (s, 12H, 4×Ar—CH₃), 2.30 (s, 6H, 2×Ar—CH₃), 3.56 (s, 2H, —CH═CH—), 6.96 (s, 4H, 4×Ar—H), 8.50 (s, 2H, 2×—CH═NAr).

### Example 6

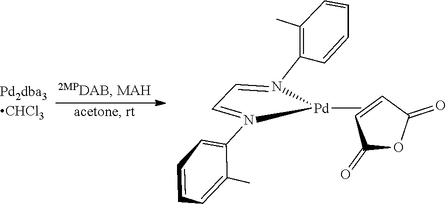

This synthesis was conducted under ambient atmosphere using commercial materials as-received. A 250 mL round bottom flask containing a stirbar was charged with Pd2dba3·CHCl₃ (250.0 mg, 0.242 mmol), N,N′-bis(2-methylphenyl)ethan-1,2-diimine (119.9 mg, 0.510 mmol, 2.1 equivalents), and maleic anhydride (49.7 mg, 0.510 mmol, 2.1 equivalents). Acetone (40 mL) was added to dissolve/suspend the components. The reaction mixture was stirred at room temperature for three hours, producing a dark purple/red homogeneous solution. The reaction solution was concentrated under vacuum to approximately 2 mL. TBME (40 mL) was added and the solution was stirred for 15 minutes, producing a purple/red slurry. After the solid settled, the TBME was decanted, leaving a purple/red solid. The solid was dissolved in a minimum of acetone and filtered through Celite to remove palladium black. The Celite bed was thoroughly rinsed with acetone until the rinses were colorless, and the combined filtrate was evaporated under vacuum. The resulting purple/red solid was washed with TBME (7×10 mL) and dried under vacuum to give the desired complex.

### Example 7

In this example the stability of complex 1 over time in both the solid-state (for long term storage) and in solution (for manual or automated solution dispensing) was evaluated. Complex 1 is stable in the solid-state for at least 6 months at room temperature under ambient atmosphere, with no discernable change to the NMR spectra over this time. Prior studies of Pd(0) complexes of this type describe rapid solution decomposition to metallic Pd, which would severely hamper its use in solution-based dispensing to high-throughput screening plates. The concentration of complex 1 was therefore monitored over time at room temperature by ¹H NMR spectroscopy versus an internal standard using 5 different deuterated solvents and a concentration of complex 1 of 20 mg/mL.

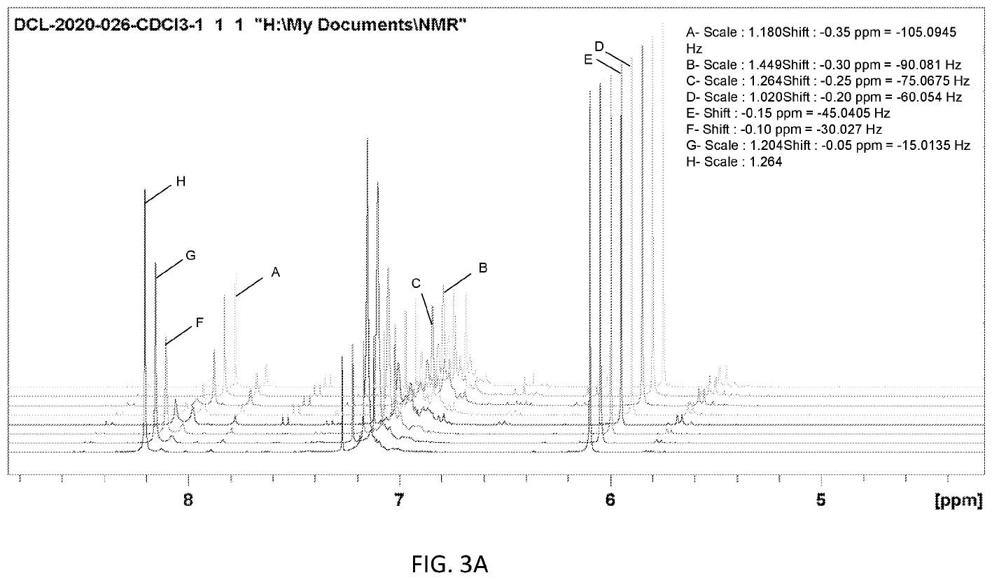

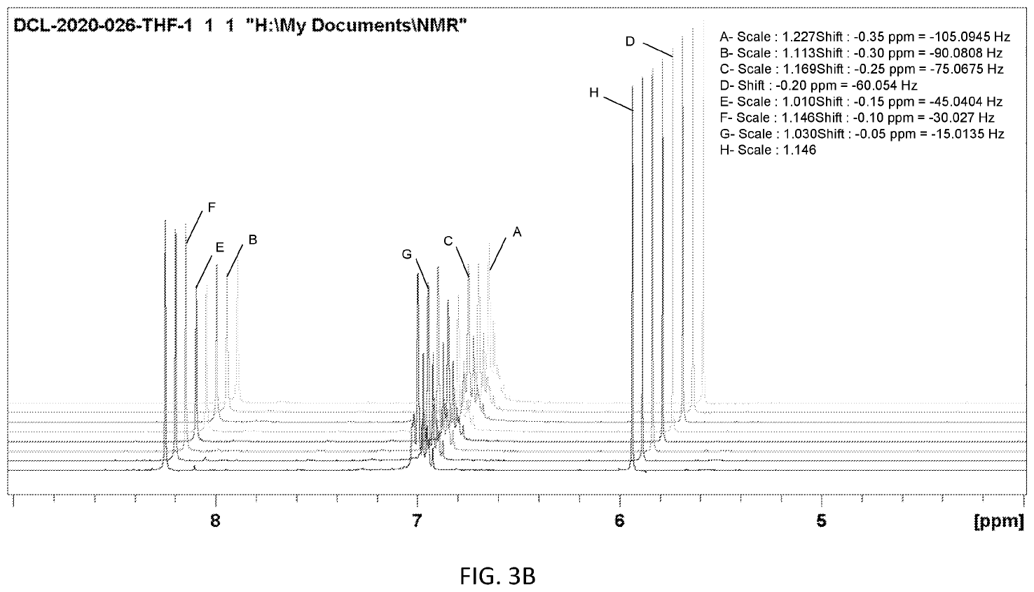

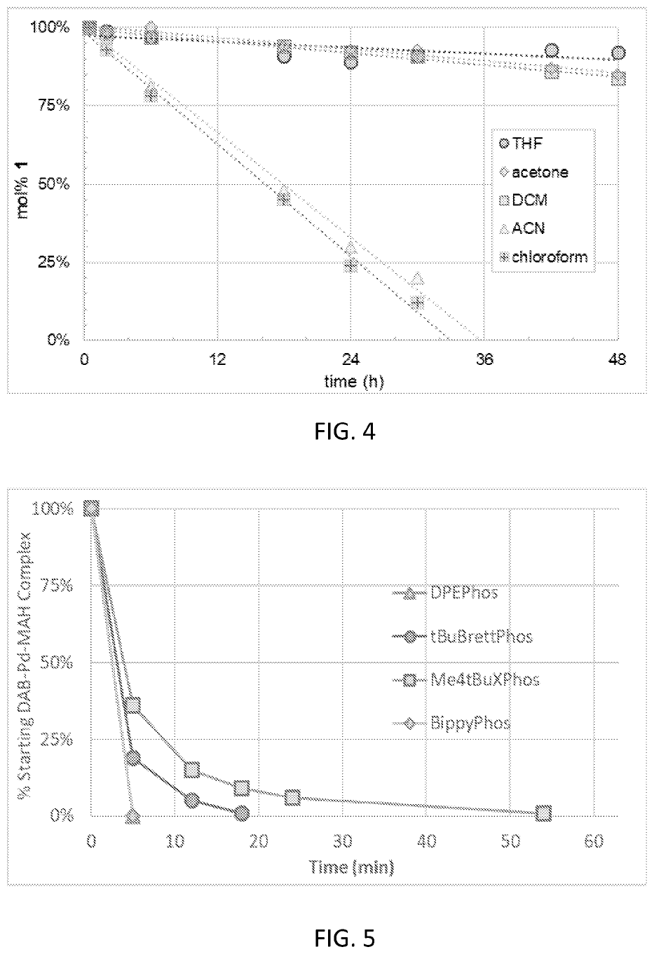

Solution Stability—Complex 1 (12.0 mg, 0.0256 mmol) was dissolved in six different deuterated solvents (CDCl₃, CD₂Cl₂, d₆-acetone, CD₃CN, d₆-DMSO, and d₈-THF; 0.6 mL each) to generate solutions of 20 mg 1/1 mL solvent. Complex 1 is not soluble in d₆-DMSO, so this solvent was omitted from the stability analysis. 1,3,5-Trimethoxybenzene (˜3 mg) was added to each solution as an internal standard. Initial ¹H NMR spectra were obtained for each solution after 30 minutes (300 MHz), and the peak area ratio for the imine C—H signal (8.43 ppm in d₆-acetone) and the internal standard Ar—H signal was recorded. Subsequent ¹H NMR spectra were obtained at 2, 6, 18, 24, 30, 42, and 48 hours; FIGS. 3A and 3B contain representative stack plots for spectra obtained in CDCl₃ (FIG. 3A) and d₆-THF (FIG. 3B). The peak area ratio for the imine C—H signal and the internal standard Ar—H signal for each spectrum was divided by the initial ratio to generate the normalized concentration data in Table 1. For CDCl₃ and CD₃CN, a palladium mirror was clearly visible on the inside wall of the NMR tubes after <18 hours, whereas the NMR tubes containing the CD₂Cl₂, d₆-acetone, and d₈-THF solutions remained mirror-free over 48 hours. FIG. 4 shows a graph plotting the results observed for this example.

TABLE 1
Normalized [complex 1] (starting from an initial
concentration of 20 mg/mL) for solutions using five different
deuterated solvents over 48 hours to assess stability.

| Time | | | [1]/[1]_{30 min} | | |
|---|---|---|---|---|---|
| (h) | CDCl₃ | CD₂Cl₂ | d₆-acetone | CD₃CN | d₆-THF |
| 0.5 | 1.00 | 1.00 | 1.00 | 1.00 | 1.00 |
| 2 | 0.93 | 0.98 | 0.99 | 0.97 | 0.99 |
| 6 | 0.78 | 0.97 | 1.00 | 0.81 | 0.97 |
| 18 | 0.45 | 0.94 | 0.94 | 0.48 | 0.91 |
| 24 | 0.24 | 0.92 | 0.93 | 0.30 | 0.89 |
| 30 | 0.12 | 0.91 | 0.93 | 0.20 | 0.91 |
| 42 | n/d | 0.86 | 0.87 | n/d | 0.93 |
| 48 | n/d | 0.84 | 0.85 | n/d | 0.92 |

In both acetonitrile and chloroform, rapid decomposition was observed for some embodiments. In some examples, only 25-30% of complex 1 remains after 24 hours, and a Pd mirror was observed. Complex 1 is quite stable in THF, acetone, and DCM. After 6 hours, a typical length of time for a single plate solution dispense, the amount of 1 remaining in these solvents is 97-100%. After 24 hours, the length of a longer automated multiplate dispense, 89-93% of complex 1 remains. In some examples, complex 1 is used as freshly-prepared THF solutions for dispensing to HTE plates.

### Example 8

In this example, six (MAH)Pd(phosphine) were synthesized. Given the simple synthesis and stability of complex 1, its suitability as a precursor for in situ catalyst formation during THE was evaluated. In some embodiments, it is desirable for phosphine metallation to Pd(0) to be rapid at room temperature to give a single, well-defined species. This can ensure self-consistent results across a screening plate that do not depend on secondary factors such as catalyst activation. In this example, complex 1 was mixed with 2 molar equivalents of a variety of mono and bidentate phosphines in d₆-acetone and monitored to the rate and extent of ligand substitution by ¹H and ³¹P NMR spectroscopy. In every case, rapid and complete conversion to a single new ³¹P-containing species was observed, even for extremely large cone angle ligands.

Based on these results, a series of 13 phosphine-based Pd(0) catalysts (catalyst 2-14) were prepared using a set of ligands commonly used in cross-coupling catalysis and a novel Pd(0) precursor disclosed herein. All of these syntheses were carried out at room temperature in THF, and the products isolated by simple precipitation under ambient atmosphere. The prepared catalysts ranged from the dppp-Pd-MAH (see catalyst 2) to those with large biaryl and bispyrazolyl ligands, including tBuBrettPhos (see catalyst 11), tBuBippyPhos (see catalyst 12), Me₄tBuXPhos (see catalyst 13), and JackiePhos (see catalyst 14). These complexes are effective as single-component Pd(0) precursor complexes for a variety of Pd-catalyzed reactions.

Unless otherwise noted, the following general procedure was used to prepare the following phosphine-Pd-MAH complexes: Starting materials were handled and weighed under inert dinitrogen atmosphere in the glovebox (due to oxygen-sensitivity of the phosphines). A 20 mL vial or 50 mL round-bottom flask was charged with complex 1 (100.0 mg, 0.2133 mmol), the corresponding phosphine ligand (1.05 equiv, 0.2240 mmol), and a cross-shaped magnetic stirbar. Anhydrous, degassed THF (5-15 mL) was added, and the reaction mixture stirred for 1-2 hours. During this time, the solution changes colour from an initial dark red/purple to yellow/orange; the exact final colour and the rate of colour change depends on the phosphine used. After the 1-2 hours stirring, the solution can be opened to ambient atmosphere if desired. The solvent was then removed in vacuo to give a yellow to orange residue. This residue was triturated with hexanes or diethyl ether (2-4 mL), followed by decantation of the liquid phase (with or without centrifugation as required). This trituration/decantation process was repeated 2-5 more times to remove the byproduct, as well as any excess phosphine. The solid was then dried in vacuo to give the product.

Additionally, the ligand substitution progress for four phosphines reacting with complex 1 at room temperature was followed by ¹H NMR spectroscopy using the following procedure. Inside a nitrogen glovebox, a 1.8 mL HPLC vial was charged with ligand (0.0134 mmol for DPEPhos and DIPP-NHC; 0.0271 mmol for monodentate phosphines) and d₈-THF (0.2 mL). The vial was sealed with an aluminum crimp cap containing a PTFE-lined rubber septum and removed from the glovebox. An NMR tube was charged with ^{DMP}DAB-Pd-MAH (1, 6.3 mg, 0.0134 mmol) and 1,3,5-trimethoxybenzene (˜0.0134 mmol) with a total d₈-THF volume of 0.5 mL, and the tube capped with a screw-cap containing a septum. An initial ¹H NMR spectrum was obtained to lock and shim on the sample, and to establish the relative integration between the signals for the starting Pd complex and the internal standard. The solution of phosphine was then added to the NMR tube via syringe, and ¹H NMR spectra were taken at regular intervals to monitor the reaction progress (FIG. 5).

### Example 8A

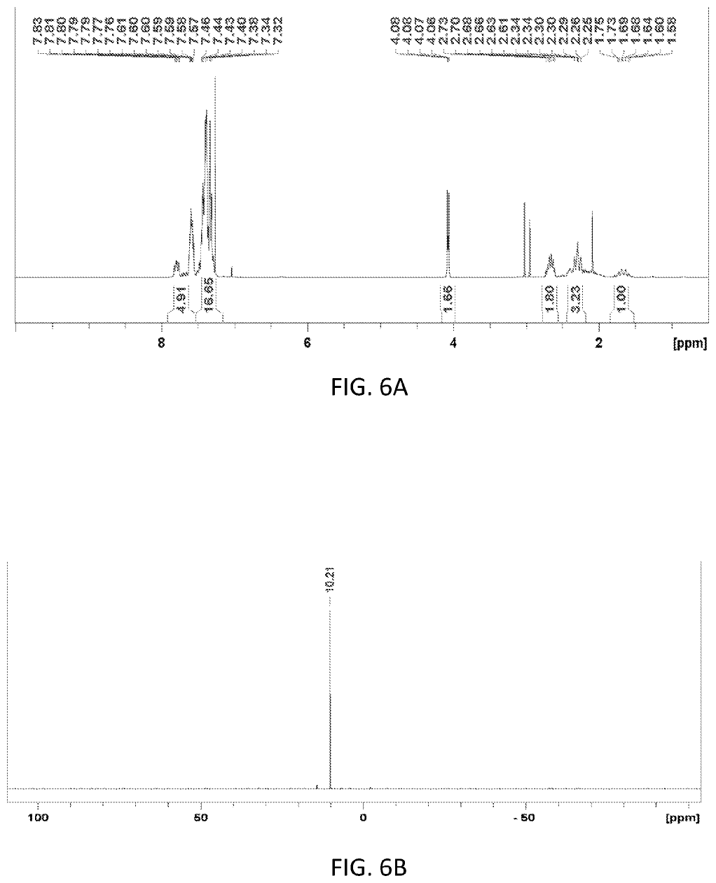

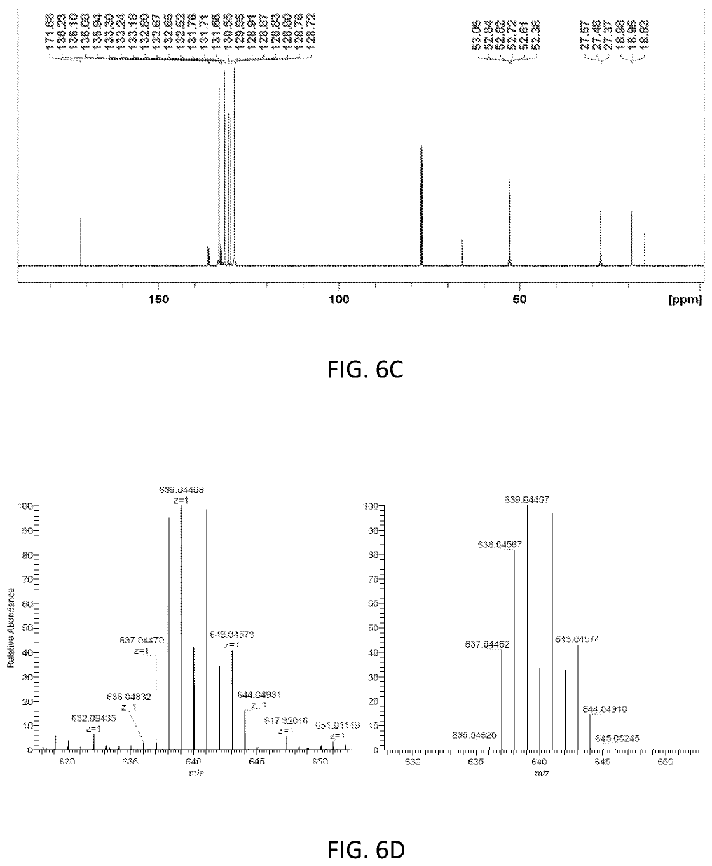

dppp-Pd-MAH (catalyst 2)—Prepared according to the general procedure using complex 1 (123.0 mg, 0.2623 mmol), dppp (113.6 mg, 0.2755 mmol), and THE (5 mL). Trituration/decantation 3× with diethyl ether (4 mL). Tan solid: 130.0 mg (80%). ¹H NMR: (300 MHz; CDCl₃) δ 1.58-1.75 (m, 1H, dppp tether), 2.25-2.34 (m, 3H, dppp-tether), 2.61-2.73 (m, 2H, dppp tether), 4.07 (m, 2H, —CH═CH—), 7.32-7.46 (m, 16H, Ar—H), 7.57-7.83 (m, 4H, Ar—H); ¹³C{¹H} NMR: (125 MHz; CDCl₃) δ 18.9 (t, J=3.8 Hz), 27.5 (m), 52.7 (m), 128.8 (t, J=5.0 Hz), 128.9 (t, J=5.0 Hz), 130.0, 130.6, 131.7 (t, J=6.3 Hz), 132.7 (m), 133.2 (t, J=7.5 Hz), 136.1 (m), 171.6; ³¹P{¹H} NMR: (121 MHz; CDCl₃) δ 10.2. HRMS (ESI) of [C₃₁H₂₈O₃P₂Pd·Na]⁺ (major isotopomer, sodium adduct): 639.04407 (calc'd); 639.04408 (found). Spectra are shown in FIGS. 6A-6D.

### Example 8B

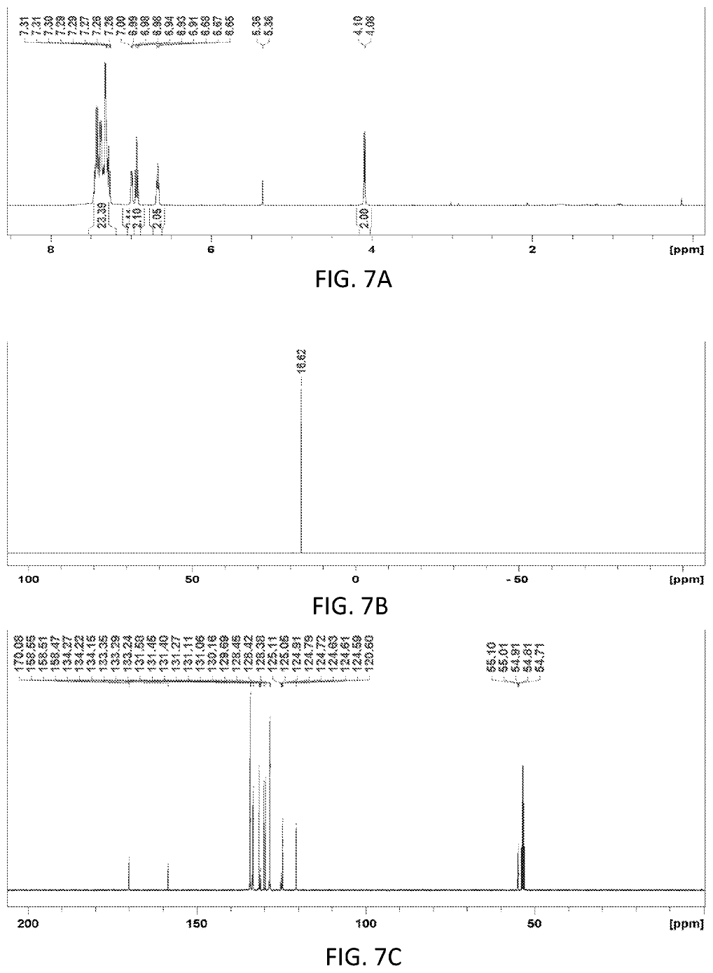

DPEPhos-Pd-MAH (catalyst 3)—Prepared according to the general procedure using complex 1 (100.0 mg, 0.2133 mmol), DPEPhos (120.6 mg, 0.2240 mmol), and THF (5 mL). Trituration/decantation 3× with diethyl ether (4 mL). Tan solid: 135.7 mg (86%). Crystals for X-ray diffraction were grown at room temperature from DCM/Et₂O (Et₂O as anti-solvent) by layering Et₂O on top of a concentrated solution of catalyst 3 in DCM. ¹H NMR: (500 MHz; CD₂Cl₂) δ 4.09 (m, 2H, —CH═CH—), 6.67 (m, 2H, Ar—H), 6.93 (t, 2H, J=7.6 Hz), 6.99 (dd, 2H, J=3.7, 8.4 Hz), 7.26-7.46 (m, 24H, Ar—H); ¹³C{¹H} NMR: (125 MHz; CD₂Cl₂) δ 54.9 (m), 120.6, 124.6 (t, J=2.6 Hz), 124.9 (m), 128.4 (t, J=4.7 Hz), 129.7, 130.2, 131.3 (m), 131.6, 133.3 (t, J=6.8 Hz), 133.4 (m), 134.2 (t, J=7.5 Hz), 158.5 (t, J=4.8 Hz), 170.1; ³¹P{¹H} NMR: (202 MHz; CD₂Cl₂) b 16.6. HRMS (ESI) of [C₄₀H₃₀O₄P₂Pd·Na]⁺ (major isotopomer, sodium adduct): 765.05463 (calc'd); 765.05497 (found). Spectra are shown in FIGS. 7A-7D.

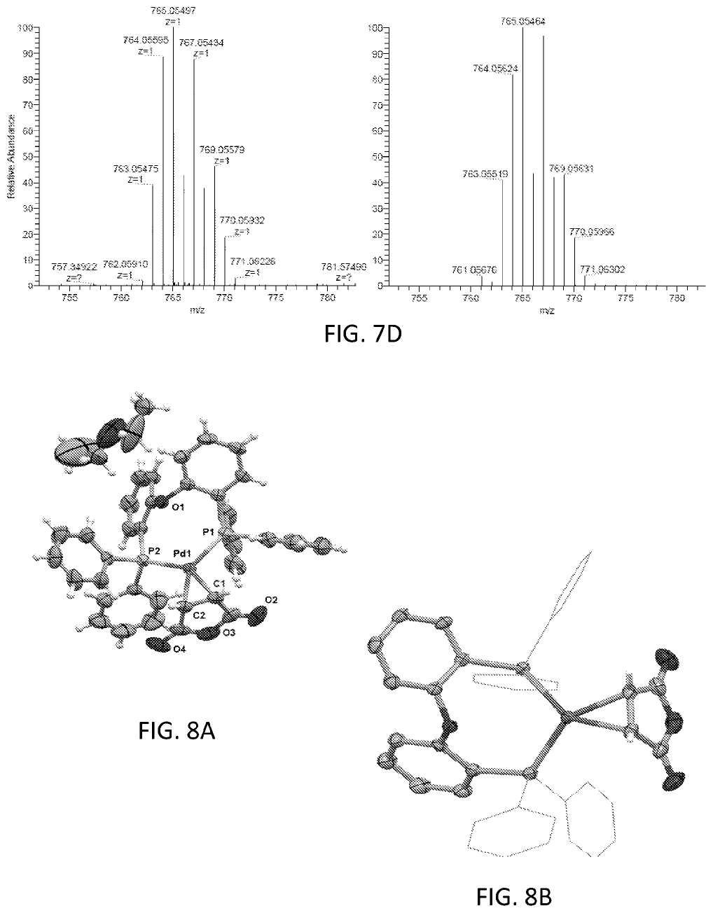

Single crystals of C₄₄H₄₀O₅P₂Pd [catalyst 3·Et₂O] were selected using a MiteEGen loop using paratone oil. A suitable crystal was selected run on a Bruker APEX-II CCD diffractometer. The crystal was kept at 273.15 K during data collection. Using Olex2, the structure was solved with the olex2.solve structure solution program using Charge Flipping and refined with the XL refinement package using Least Squares minimization. FIG. 8A shows the solid-state molecular structure of catalyst 3, including diethyl ether solvate and FIG. 8B shows the solid-state molecular structure determined by single crystal XRD wherein hydrogen atoms omitted for clarity, except those on the maleic anhydride ligand and phosphine substituents are shown in wireframe for clarity.

Crystal structure determination of [catalyst 3] for C₄₄H₄₀O₅P₂Pd (M=817.10 g/mol): triclinic, space group P-1 (no. 2), a=11.0488(7) Å, b=12.4133(8) Å, c=15.6581(10) Å, α=99.623(2)°, β=106.450(2)°, γ=105.981(2)°, V=1908.8(2) Å³, Z=2, T=273.15 K, μ(MoKα)=0.615 mm⁻¹, Dcalc=1.422 g/cm³, 75385 reflections measured (5.074°≤2Θ≤56.036°), 9217 unique (Rᵢₙₜ=0.0824, R_{sigma}=0.0586) which were used in all calculations. The final R₁ was 0.0541 (I>2σ(I)) and wR₂ was 0.1133 (all data).

### Example 8C

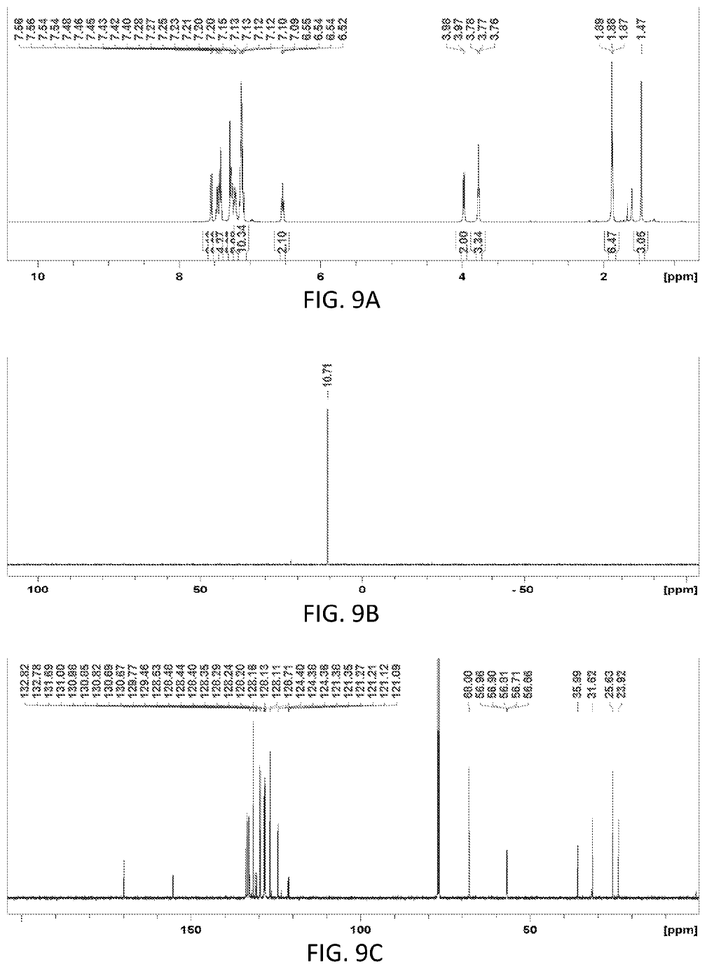

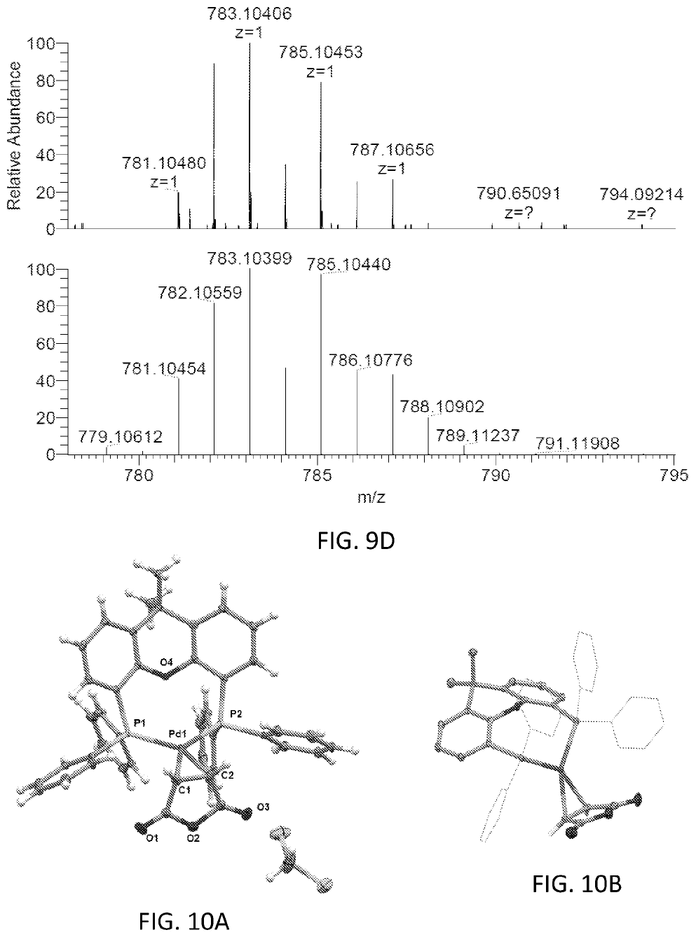

XantPhos-Pd-MAH (catalyst 4)—prepared according to the general procedure using complex 1 (100.0 mg, 0.2133 mmol), XantPhos (129.6 mg, 0.2240 mmol), and THF (5 mL). Trituration/decantation 3× with THF (4 mL). White solid: 147.7 mg (81%). Catalyst isolated as the THF solvate (1 equivalent THF, as observed by ¹H NMR spectroscopy, remains after extensive vacuum drying of the isolated solid). Crystals for X-ray diffraction were grown at room temperature from DCM/Et₂O (Et₂O as anti-solvent) by layering Et₂O on top of a concentrated solution of catalyst 4 in DCM. ¹H NMR: (500 MHz; CDCl₃) δ 1.47 (s, 3H, —CH₃), 1.87 (m, 4H, THF), 1.89 (s, 3H, —CH₃), 3.77 (m, 4H, THF), 3.98 (m, 2H, —CH═CH—), 6.54 (td, 2H, J=1.0, 8.0 Hz), 7.09-7.28 (m, 16H, Ar—H), 7.42 (t, 4H, J=7.4 Hz), 7.46 (t, 2H, J=7.4 Hz), 7.55 (dd, 2H, J=1.0, 8.0 Hz); ¹³C{¹H} NMR: (125 MHz; CDCl₃) δ 23.9, 25.6 (THF), 31.6, 36.0, 56.8 (m), 68.0 (THF), 121.2 (m), 124.4 (t, J=2.6 Hz), 126.7, 128.2 (m), 128.4 (m), 129.5, 129.8, 130.8 (m), 131.7, 132.9 (m), 133.5 (m), 133.9 (m), 155.4 (t, J=5.2 Hz), 169.8; ³¹P{¹H} NMR: (202 MHz; CDCl₃) δ 10.7. HRMS (ESI) of [C₄₃H₃₄O₄P₂Pd·H]⁺ (major isotopomer, proton adduct): 783.10399 (calc'd); 783.10406 (found). Spectra are shown in FIGS. 9A-9D.

Single crystals of C_{43.5}H₃₅ClO₄P₂Pd [catalyst 4.0.5DCM] were selected using a MitEGen loop and paratone oil. A suitable crystal was selected and run on a Bruker APEX-II CCD diffractometer. The crystal was kept at 99.99 K during data collection. Using Olex2, the structure was solved with the olex2.solve structure solution program using Charge Flipping and refined with the XL refinement package using Least Squares minimization. FIG. 10A shows the solid-state molecular structure of catalyst 4, including diethyl ether solvate and FIG. 10B shows the solid-state molecular structure determined by single crystal XRD wherein hydrogen atoms omitted for clarity, except those on the maleic anhydride ligand and phosphine substituents are shown in wireframe for clarity.

Crystal structure determination of [catalyst 4.0.5DCM] for C_{43.5}H₃₅ClO₄P₂Pd (M=825.50 g/mol): monoclinic, space group C2/c (no. 15), a=24.7326(18) Å, b=13.2010(8) Å, c=23.4604(17) Å, β, =111.779(2)°, V=7113.0(9) Å³, Z=8, T=99.99 K, μ(MoKα)=0.732 mm⁻¹, Dcalc=1.542 g/cm³, 34983 reflections measured (3.546°≤2Θ≤55.946°), 8524 unique (Rᵢₙₜ=0.0645, R_{sigma}=0.0608) which were used in all calculations. The final R₁ was 0.0384 (I>2σ(1)) and wR₂ was 0.0830 (all data).

### Example 8D

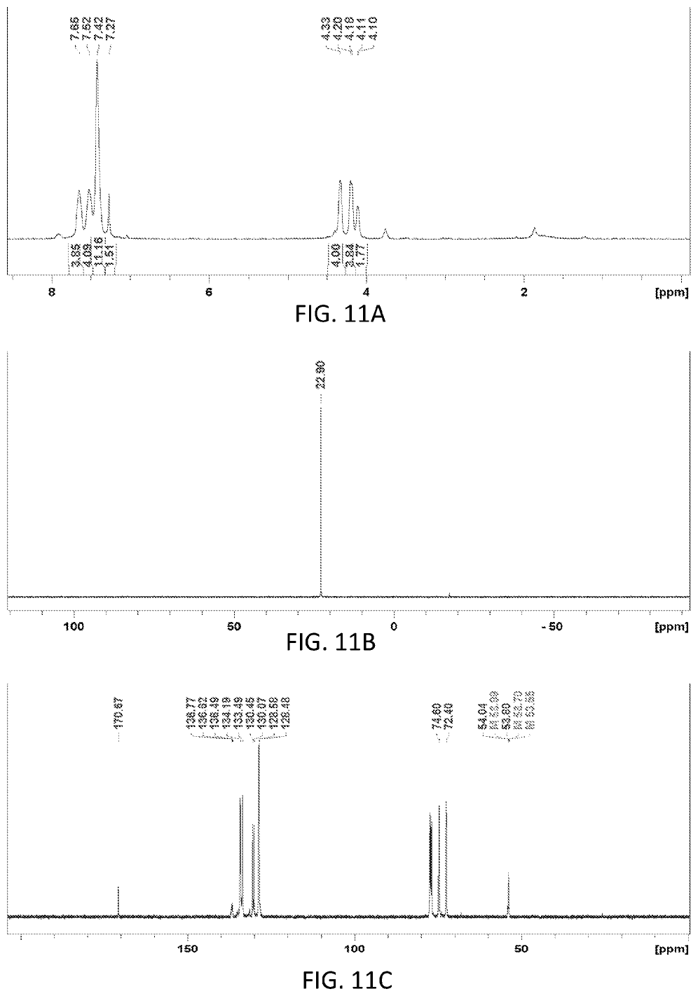

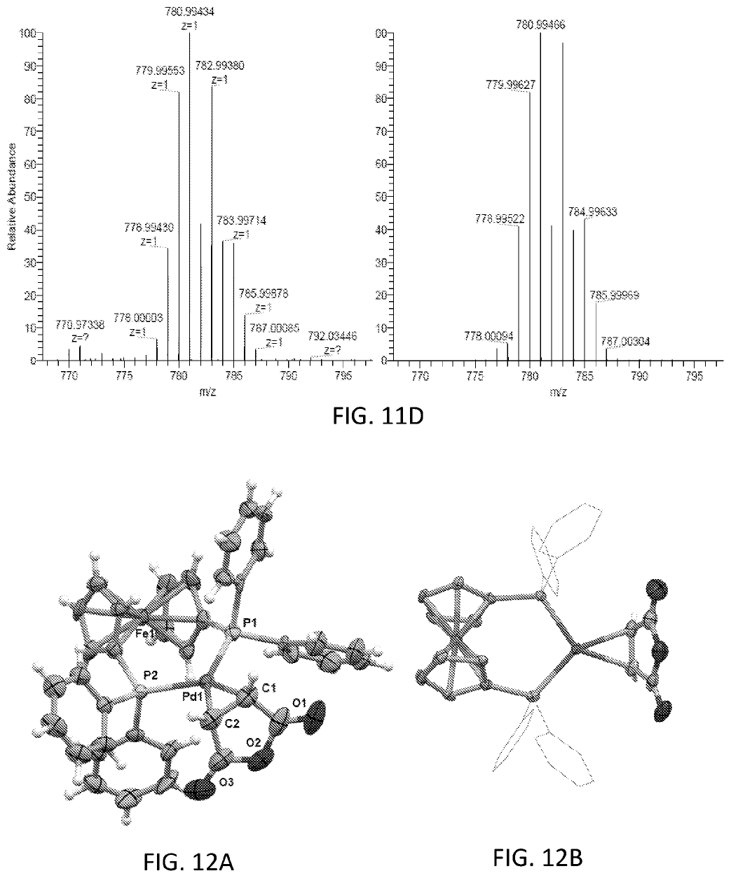

dppf-Pd-MAH (catalyst 5)—Prepared according to the general procedure using complex 1 (100.0 mg, 0.2133 mmol), dppf (124.2 mg, 0.2240 mmol), and THE (5 mL). Trituration/decantation 3× with diethyl ether (4 mL). Brown solid: 150.0 mg (93%). Crystals for X-ray diffraction were grown at room temperature from DCM/Et₂O (Et₂O as anti-solvent) by layering Et₂O on top of a concentrated solution of catalyst 5 in DCM. ¹H NMR: (300 MHz; CDCl₃) δ 4.11 (br m, 2H, —CH═CH—), 4.19 (br m, 4H, Cp-H), 4.33 (br m, 4H, Cp-H), 7.42 (br m, 12H, Ph-H), 7.52 (br m, 4H, Ph-H), 7.65 (br m, 4H, Ph-H); ¹³C{¹H} NMR: (125 MHz; CDCl₃) δ 53.8 (m), 72.4, 74.6, 128.5 (d, J=13.4 Hz), 130.2 (d, J=48.0 Hz), 133.8 (m), 136.6 (m), 170.7; ³¹P{^{1H}}NMR: (202 MHz; CDCl₃) δ 22.9. HRMS (ESI) of [C₃₈H₃₀FeO₃P₂Pd—Na]+(major isotopomer, Na⁺ adduct): 780.99466 (calc'd); 780.99434 (found). Spectra are shown in FIGS. 11A-11D.

Single crystals of C₃₈H₃₀FeO₃P₂Pd [catalyst 5] were selected using a MiteEGen loop using paratone oil. A suitable crystal was selected and run on Bruker APEX-II CCD diffractometer. The crystal was kept at 273.15 K during data collection. Using Olex2, the structure was solved with the olex2.solve structure solution program using Charge Flipping and refined with the XL refinement package using Least Squares minimization. FIG. 12A shows the solid-state molecular structure of catalyst 5, including diethyl ether solvate and FIG. 12B shows the solid-state molecular structure determined by single crystal XRD wherein hydrogen atoms omitted for clarity, except those on the maleic anhydride ligand and phosphine substituents are shown in wireframe for clarity.

Crystal structure determination of [catalyst 5] for C₃₈H₃₀FeO₃P₂Pd (M=758.81 g/mol): monoclinic, space group Cc (no. 9), a=26.339(12) Å, b=8.380(4) Å, c=17.649(9) Å, β=124.36(2)°, V=3216(3) Å³, Z=4, T=273.15 K, μ(MoKα)=1.147 mm⁻¹, Dcalc=1.567 g/cm³, 52738 reflections measured (5.21°≤2Θ≤56.218°), 7730 unique (Rᵢₙₜ=0.1286, R_{sigma}=0.1243) which were used in all calculations. The final R₁ was 0.1147 (I>2σ(I)) and wR₂ was 0.1548 (all data).

### Example 8E

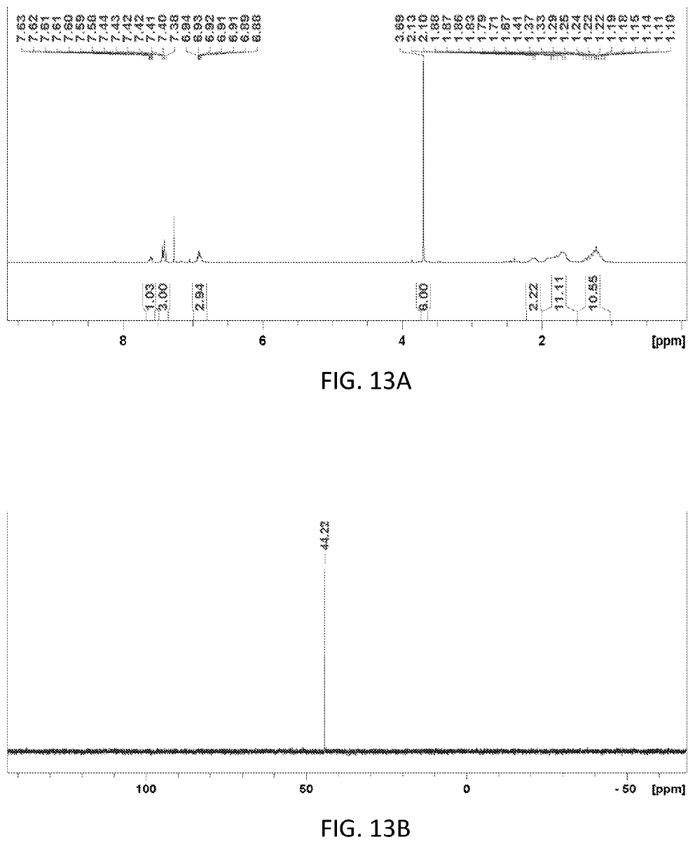

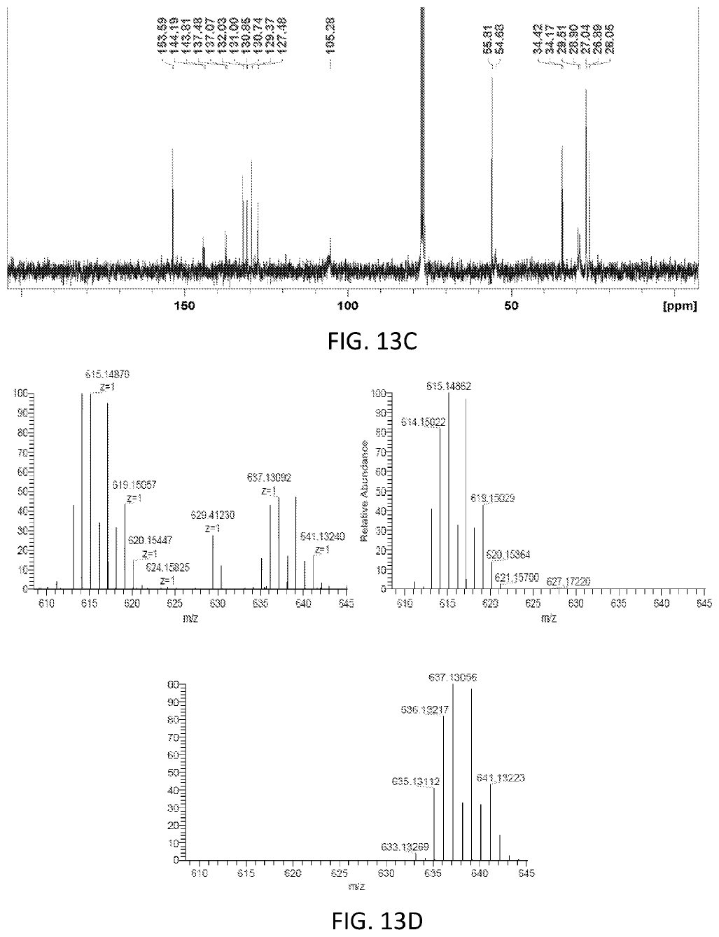

SPhos-Pd-MAH (catalyst 6)—Prepared according to the general procedure using complex 1 (100.0 mg, 0.2133 mmol), SPhos (91.9 mg, 0.2240 mmol), and THF (5 mL). Trituration/decantation 3× with diethyl ether (4 mL). Yellow solid: 86.6 mg (66%). ¹H NMR: (300 MHz; CDCl₃) δ 1.10-1.40 (m, 10H, Cy-H), 1.60-1.96 (br m, 10H, Cy-H), 2.12 (br m, 2H, Cy-H), 3.69 (s, 6H, 2×OCH₃), 4.5 (v br, 1H, —CH═CH—), 6.91 (m, 3H, Ar—H), 7.37-7.46 (m, 3H, Ar—H), 7.60 (m, 1H, Ar—H); ¹³C{¹H} NMR: (75 MHz; CDCl₃) δ 26.1, 27.0 (d, J=11.1 Hz), 28.9 (m), 29.5 (br), 34.3 (d, J=19.0), 54.7 (br), 55.8, 105.3 (br), 127.5 (d, J=4.0 Hz), 129.4, 130.7 (d, J=2.1 Hz), 130.9 (d, J=11.1 Hz), 132.0, 137.3 (d, 31.0 Hz), 143.8, 144.2, 153.6; ³¹P{¹H} NMR: (121 MHz; CDCl₃) δ 44.2. HRMS (ESI) of [C₃₀H₃₇O₅PPd·H]⁺ (major isotopomer, H⁺ adduct): 615.14862 (calc'd); 615.14870 (found). Spectra are shown in FIGS. 13A-13D.

### Example 8F

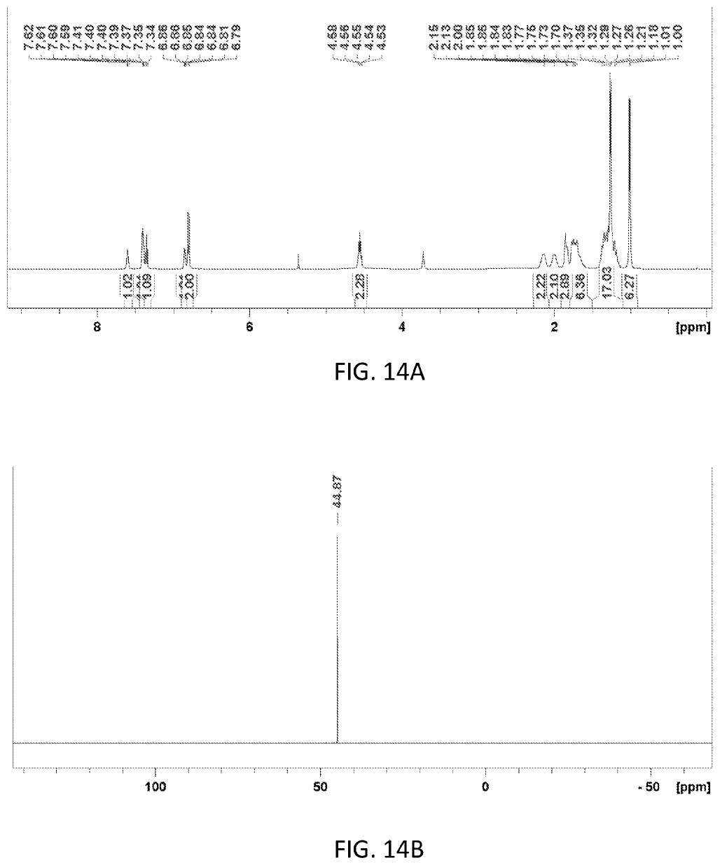

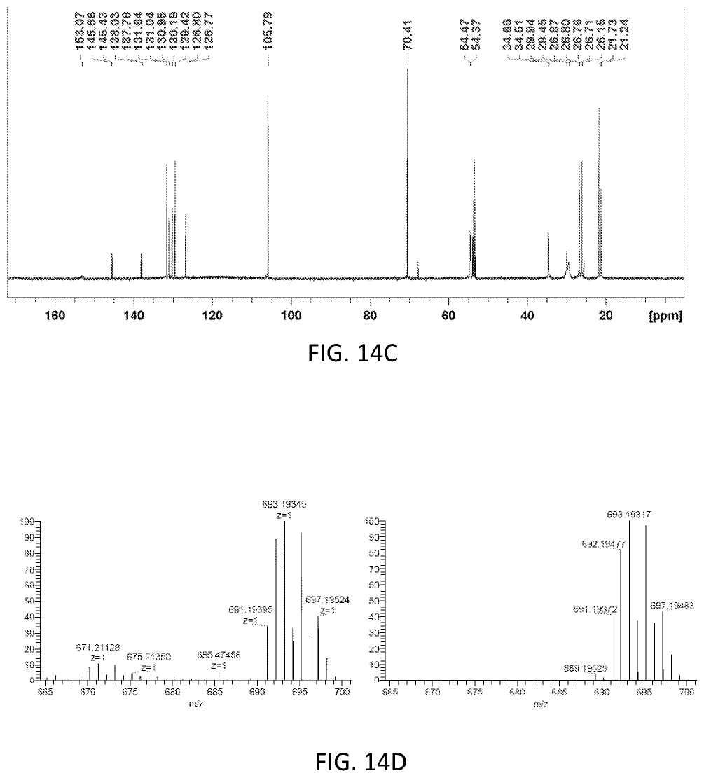

RuPhos-Pd-MAH (catalyst 7): Prepared according to the general procedure using complex 1 (99.3 mg, 0.2118 mmol), RuPhos (98.8 mg, 0.2118 mmol), and THE (15 mL). Trituration/decantation 6× with hexanes (4 mL). Yellow solid: 86.8 mg (61%). ¹H NMR: (500 MHz, CD₂Cl₂) δ 1.01 (d, 6H, J=5.9 Hz, O—CH(CH₃)₂), 1.11-1.43 (m, 10H, Cy-H), 1.26 (d, 6H, J=5.9 Hz, O—CH(CH₃)₂), 1.60-1.80 (m, 6H, Cy-H), 1.60-1.80 (m, 6H), 1.85 (m, 2H, Cy-H), 2.00 (m, 2H, Cy-H), 2.13 (m, 2H, Cy-H), 2.50 (br s, 1H, —CH═CH—), 4.50 (br s, 1H, —CH═CH—), 4.55 (sept, 2H, J=5.8 Hz, 2×O—CH(CH₃)₂), 6.80 (d, 2H, J=8.3 Hz, Ar—H), 6.83-6.89 (m, 1H, Ar—H), 7.35 (t, 1H, J=8.3 Hz, Ar—H), 7.38-7.44 (m, 2H, Ar—H), 7.57-7.63 (m, 1H, Ar—H); ¹³C{¹H} NMR: (500 MHz, CD₂Cl₂) δ 21.2, 21.7, 25.6, 26.2, 26.8 (d, J=11.1 Hz), 26.8 (d, J=13.9 Hz), 29.4, 29.9, 34.6 (d, J=19.3 Hz), 54.4, 54.5, 70.4, 105.8, 126.8 (d, J=3.8 Hz), 129.4, 130.2, 131.0 (d, J=10.8 Hz), 131.6, 137.9 (d, J=32.0 Hz), 145.5 (d, J=28.9 Hz), 153.1; ³¹P{¹H} NMR: (300 MHz, CD₂Cl₂) δ 44.9. HRMS (ESI) of [C₃₄H₄₅O₅PPd·Na]+(major isotopomer, Na⁺ adduct): 693.19317 (calc'd); 693.19345 (found). Spectra are shown in FIGS. 14A-14D.

### Example 8G

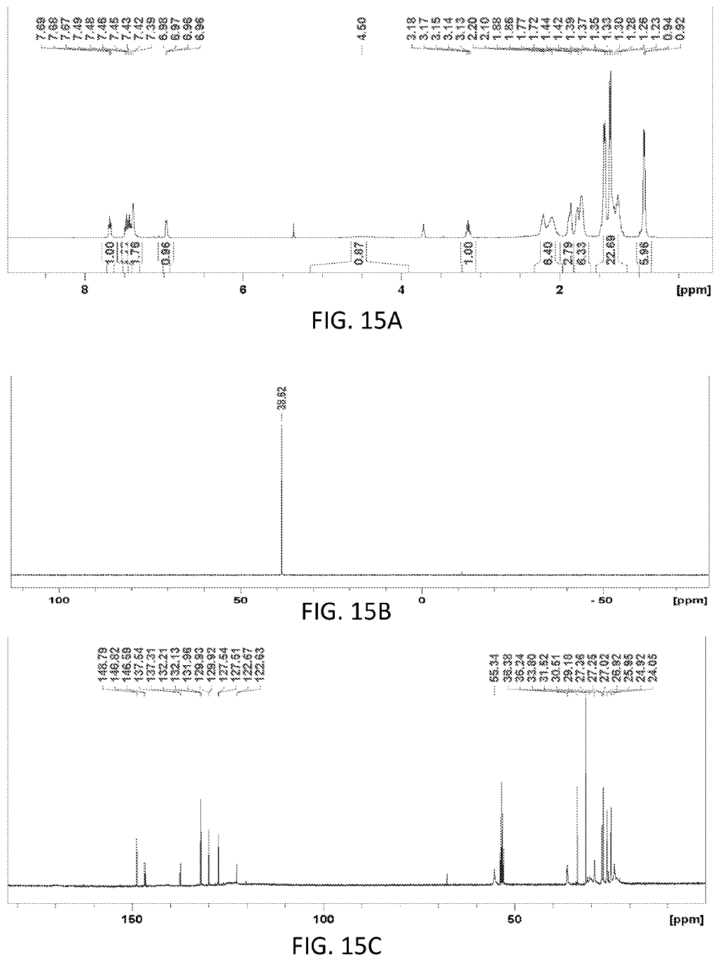

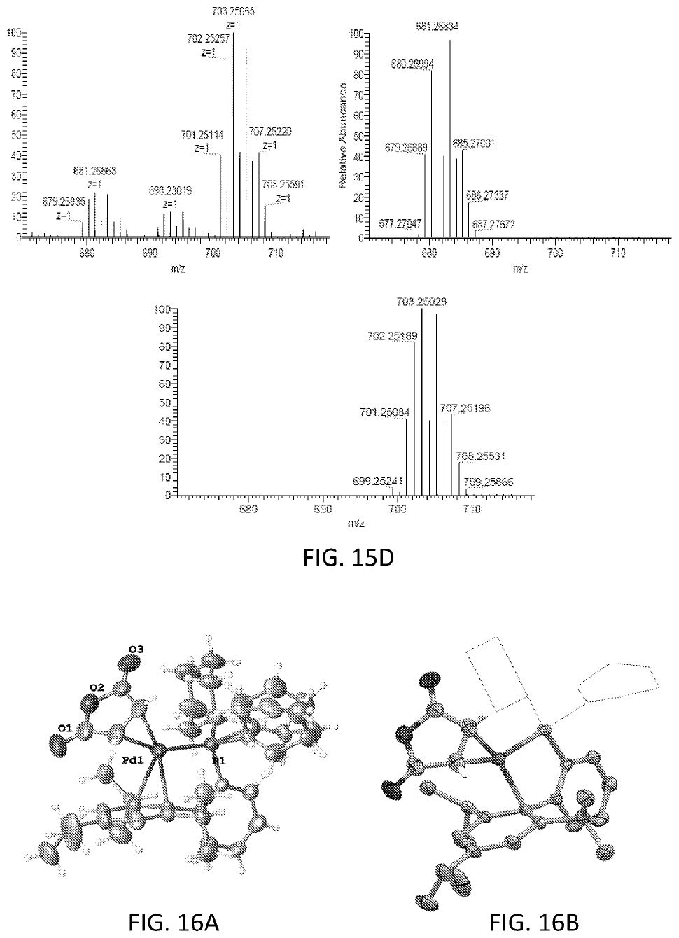

XPhos-Pd-MAH (catalyst 8): Prepared according to the general procedure using 1 (109.9 mg, 0.2344 mmol), XPhos (112.9 mg, 0.2367 mmol), and THE (10 mL). Trituration/decantation 6× with hexanes (4 mL). Yellow solid: 101.6 mg (64%). Crystals for X-ray diffraction were grown at room temperature from DCM/Et₂O (Et₂O as anti-solvent) by layering Et₂O on top of a concentrated solution of catalyst 8 in DCM. ¹H NMR: (500 MHz, CD₂Cl₂) δ 0.93 (d, 6H, J=6.3 Hz, —CH(CH₃)₂), 1.16-1.52 (m, 10H, Cy-H), 1.36 (d, 6H, J=6.7 Hz, —CH(CH₃)₂), 1.43 (d, 6H, J=6.4 Hz, —CH(CH₃)₂), 1.50-1.70 (br m, 6H, Cy-H), 1.87 (br m, 2H, Cy-H), 2.00-2.30 (br m, 6H, 2×—CH(CH₃)₂+Cy-H), 3.15 (sept, 1H, J=6.9 Hz, —CH(CH₃)₂), 4.48 (br s, 1H, —CH═CH—), 6.97 (br dd, 1H, J=2.8 Hz, 7.1 Hz, Ar—H), 7.39 (br s, 2H, Ar—H), 7.43 (t, 1H, J=7.4 Hz, Ar—H), 7.48 (t, 1H, J=7.4 Hz, Ar—H), 7.68 (t, 1H, J=6.0 Hz, Ar—H); ¹³C{¹H} NMR: 23.8, 24.0, 24.9, 26.0, 27.0 (d, J=3.0 Hz), 27.3 (d, J=3.0 Hz), 29.2, 30.3, 31.5, 33.8, 36.3 (d, J=17.7 Hz), 55.4, 122.7 (d, J=5.3 Hz), 127.5 (d, J=3.6 Hz), 129.9, 132.0, 132.2 (d, J=10.2 Hz), 137.4 (d, J=29.1 Hz), 146.7 (d, J=29.2 Hz), 148.8; ³¹P{¹H} NMR: 38.6. HRMS (ESI) of [C₃₇H₅₁O₃PPd—H]⁺ (major isotopomer, H⁺ adduct): 681.26834 (calc'd); 681.26863 (found). Spectra are shown in FIGS. 15A-15D.

Single crystals of C₃₇H₅₁O₃PPd [catalyst 8] were selected using a MiteEgen loop on a Bruker APEX-II CCD diffractometer. The crystal was kept at 273.15 K during data collection. Using Olex2, the structure was solved with the olex2.solve structure solution program using Charge Flipping and refined with the XL refinement package using Least Squares minimization. FIG. 16A shows the solid-state molecular structure of catalyst 8, including diethyl ether solvate and FIG. 16B shows the solid-state molecular structure determined by single crystal XRD wherein hydrogen atoms omitted for clarity, except those on the maleic anhydride ligand and phosphine substituents are shown in wireframe for clarity.

Crystal structure determination of [catalyst 8] for C₃₇H₅₁O₃PPd (M=680.14 g/mol): orthorhombic, space group Pbca (no. 61), a=18.282(4) Å, b=18.506(4) Å, c=20.018(4) Å, V=6773(2) Å³, Z=8, T=273.15 K, μ(MoKα)=0.629 mm⁻¹, Dcalc=1.334 g/cm³, 193553 reflections measured (5.136°≤2Θ≤52.968°), 6977 unique (Rᵢₙₜ=0.0435, R_{sigma}=0.0163) which were used in all calculations. The final R₁ was 0.0415 (I>2σ(I)) and wR₂ was 0.1104 (all data).

### Example 8H

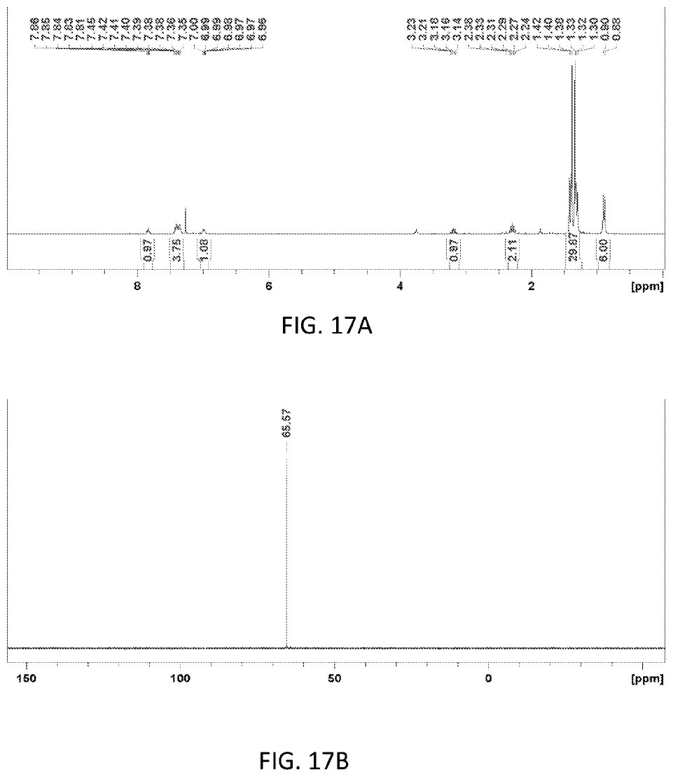

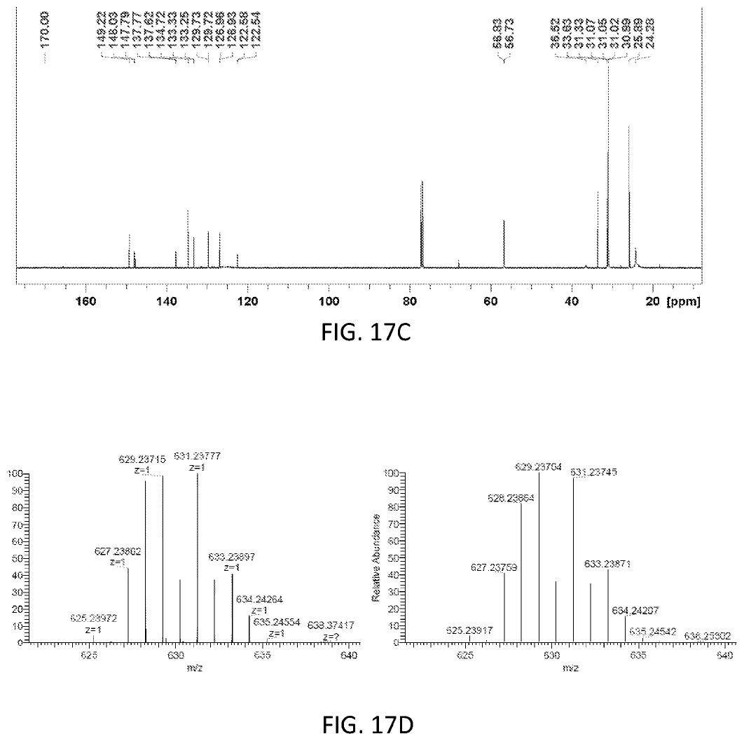

tBuXPhos-Pd-MAH (catalyst 9)—Prepared according to the general procedure using complex 1 (100.0 mg, 0.2133 mmol), tBuXPhos (95.1 mg, 0.2240 mmol), and THF (5 mL). Trituration/decantation 3× with diethyl ether (4 mL). Yellow solid: 97.0 mg (72%). ¹H NMR: (500 MHz, CDCl₃) δ 0.89 (d, 6H, J=6.6 Hz, —CH(CH₃)₂), 1.33 (d, 6H, J=6.6 Hz, —CH(CH₃)₂), 1.38 (d, 18H, J=13.9 Hz, 2×tBu), 1.42 (d, 6H, J=7.6 Hz, —CH(CH₃)₂), 2.30 (sept, 2H, J=7.0 Hz, 2×—CH(CH₃)₂), 3.20 (sept, 1H, J=7.2 Hz, —CH(CH₃)₂), 4.30 (br s, 1H, —CH═CH—), 7.00 (m, 1H, Ar—H), 7.37 (br s, 2H, Ar—H), 7.40-7.45 (m, 2H, Ar—H), 7.85 (m, 1H, Ar—H); ¹³C{¹H} NMR: 24.3 (br), 25.9, 31.0 (m), 31.3 (br), 33.6, 36.5 (br), 56.8 (d, J=13.3 Hz), 122.6 (d, J=5.0 Hz), 126.9, 127.0, 129.7 (d, J=2.1 Hz), 133.3 (d, J=10.4 Hz), 134.7, 137.7 (d, J=19.5 Hz), 147.9 (d, J=29.2 Hz), 149.2, 170.0 (v br); ³¹P{¹H} NMR: 65.6. HRMS (ESI) of [C₃₃H₄₇O₃PPd—H]⁺ (major isotopomer, H⁺ adduct): 629.23704 (calc'd); 629.23715 (found). Spectra are shown in FIGS. 17A-17D.

### Example 8I

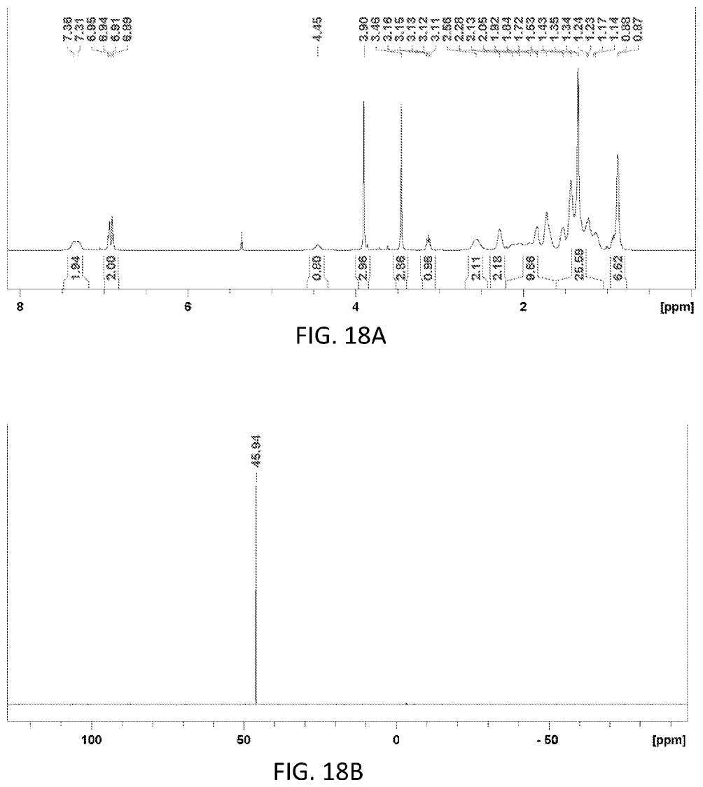

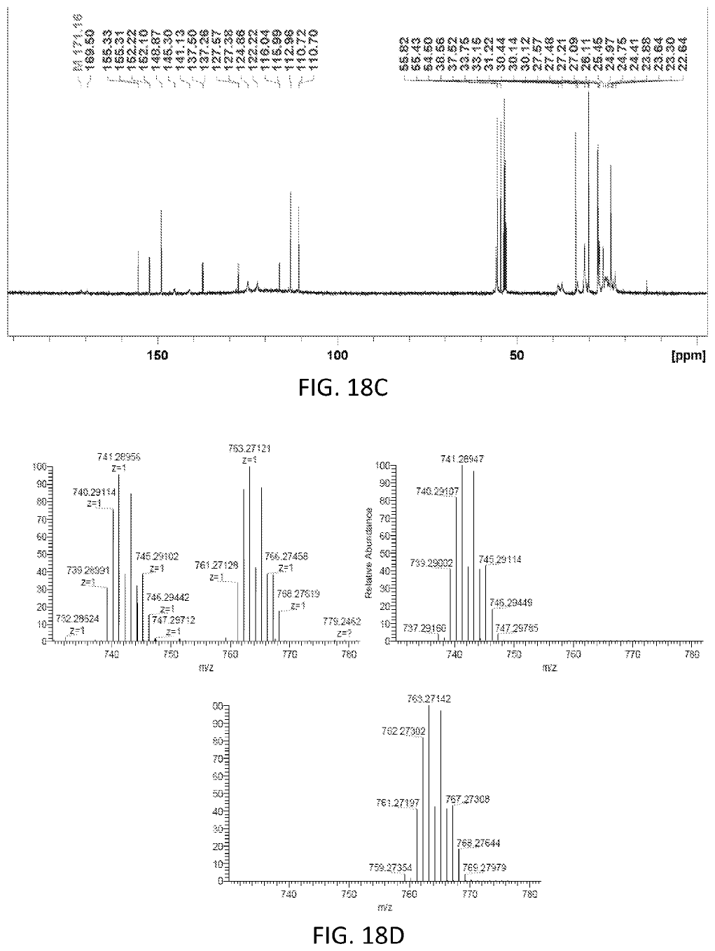

BrettPhos-Pd-MAH (catalyst 10): Prepared according to the general procedure using complex 1 (91.0 mg, 0.1941 mmol), BrettPhos (104.2 mg, 0.1941 mmol), and THF (9 mL). Trituration/decantation 5× with hexanes (5 mL). Yellow solid: 92.3 mg (64%). ¹H NMR: (500 MHz, CD₂Cl₂) δ 0.88 (br d, 6H, J=4.75 Hz), 1.04-2.21 (m, 20H, Cy-H), 1.35 (br d, 6H, J=6.7 Hz), 1.43 (br s, 6H), 2.28 (br s, 2H), 2.56 (br s, 2H), 3.13 (sept, 1H, J=6.7 Hz), 3.46 (s, 3H), 3.90 (s, 3H), 4.45 (br s, 1H), 6.83-7.00 (m, 2H), 7.21-7.50 (m, 2H); ¹³C{¹H} NMR: (125 MHz, CD₂Cl₂) δ 22.6, 22.8, 23.3, 23.6, 23.9, 24.4, 24.7, 25.0, 25.5, 26.1, 27.2 (d, J=15.4 Hz), 27.5 (d, J=11.4 Hz), 30.4, 31.2, 33.2, 33.8, 38.0 (d, J=130.5 Hz), 54.5, 55.4, 55.8, 110.7 (d, J=3.1 Hz), 113.0, 116.0 d (J=5.7 Hz), 122.2, 124.9, 127.5 (d, J=23.5 Hz), 137.3 (d, J=30.0 Hz), 141.2, 145.4, 148.8, 152.2 (d, J=14.9 Hz), 155.4 (d, J=2.5 Hz), 169.4, 171.1; ³¹P{¹H} NMR: (121 MHz, CD₂Cl₂) δ 46.0. HRMS (ESI) of [C₃₉H₅₅O₅PPd·H]⁺ (major isotopomer, H+ adduct): 741.28947 (calc'd); 741.28956 (found). Spectra are shown in FIGS. 18A-18D.

### Example 8J

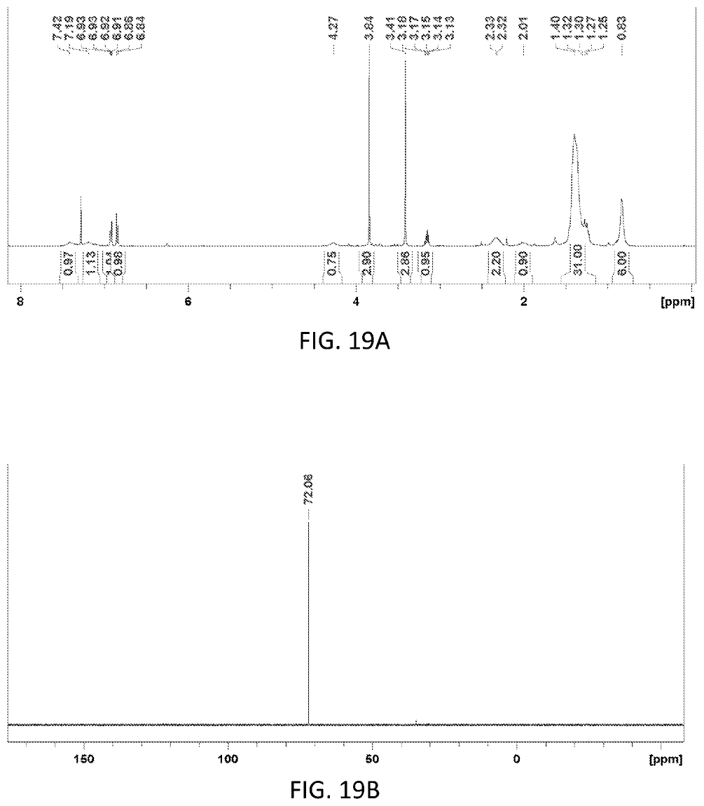

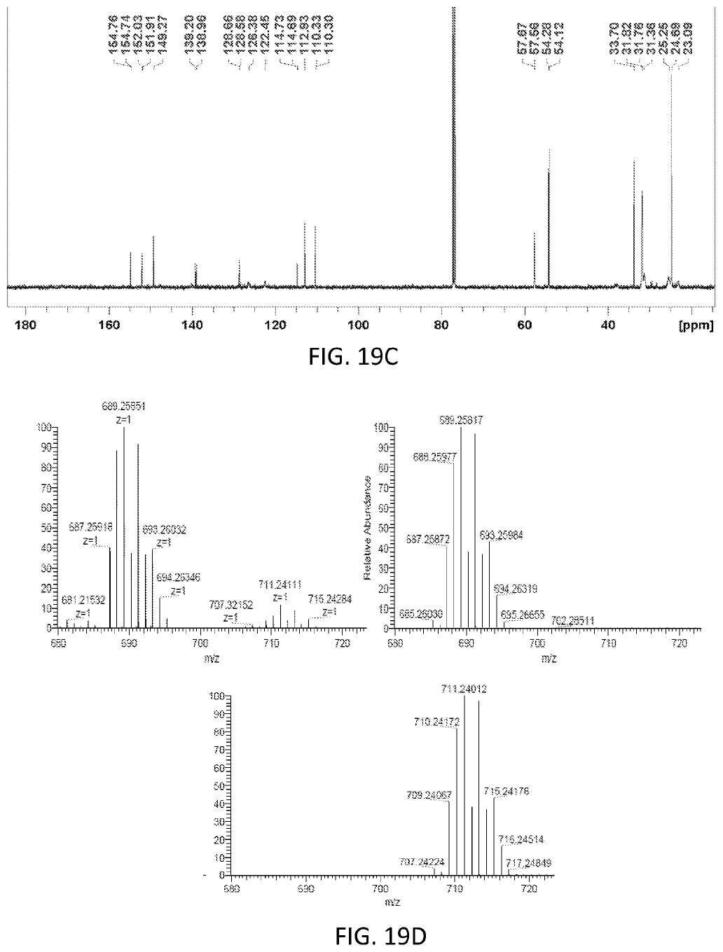

tBuBrettPhos-Pd-MAH (catalyst 11)—Prepared according to the general procedure using complex 1 (56.0 mg, 0.1194 mmol), tBuBrettPhos (60.8 mg, 0.1254 mmol), and THE (2.5 mL). Trituration/decantation 5× with hexanes (5 mL). Yellow solid: 44.0 mg (54%). ¹H NMR: (300 MHz, CDCl₃) δ 0.88 (br m, 6H), 1.20-1.50 (br m, 30H), 2.01 (br m, 1H, —CH═CH—), 3.15 (sept, 1H, J=6.8 Hz), 3.41 (s, 3H), 3.84 (s, 3H), 4.27 (br s, 1H, —CH═CH—), 6.85 (d, 1H, J=8.7 Hz), 6.93 (dd, 1H, J=1.8, 8.8 Hz), 7.19 (br s, 1H), 7.42 (br s, 1H); ¹³C{¹H} NMR: (500 MHz, CDCl₃) δ 23.1 (br), 24.7, 25.3 (br), 31.4 (br), 31.8 (d, J=7.8 Hz), 33.7, 54.1, 54.3, 57.6, 57.7, 110.3 (d, J=2.7 Hz), 112.9 (d, J=1.2 Hz), 114.7 (d, J=5.3 Hz), 122.5 (br), 126.4 (br), 128.6 (d, J=11.2 Hz), 139.1 (d, J=30.5 Hz), 149.3, 152.0 (d, J=15.2 Hz), 154.7 (d, J=2.1 Hz); ³¹P{¹H} NMR: (300 MHz, CD₂Cl₂) δ 72.1. HRMS (ESI) of [C₃₅H₅₁O₅PPd·H]⁺ (major isotopomer, H⁺ adduct): 689.25817 (calc'd); 689.25851 (found). Spectra are shown in FIGS. 19A-19D.

### Example 8K

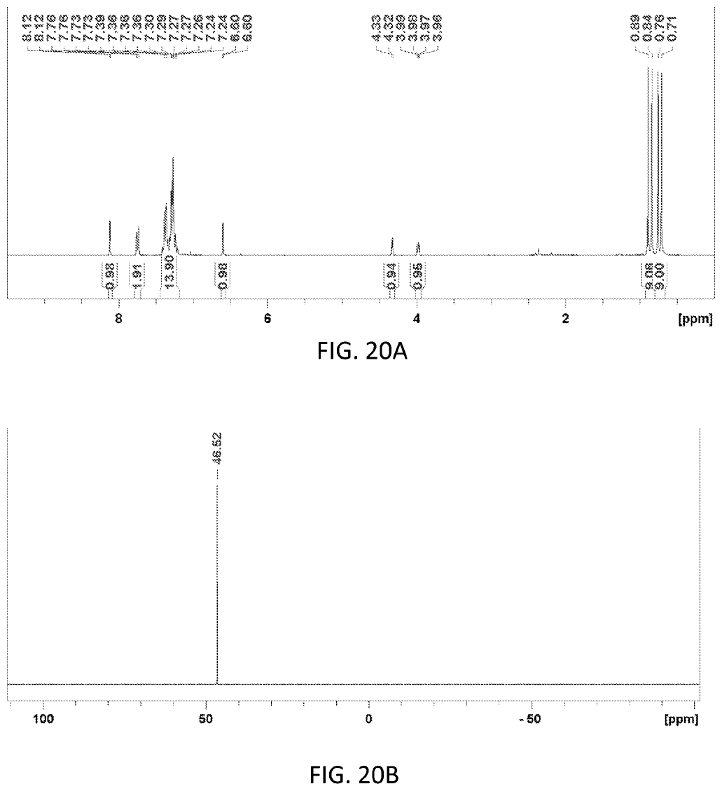

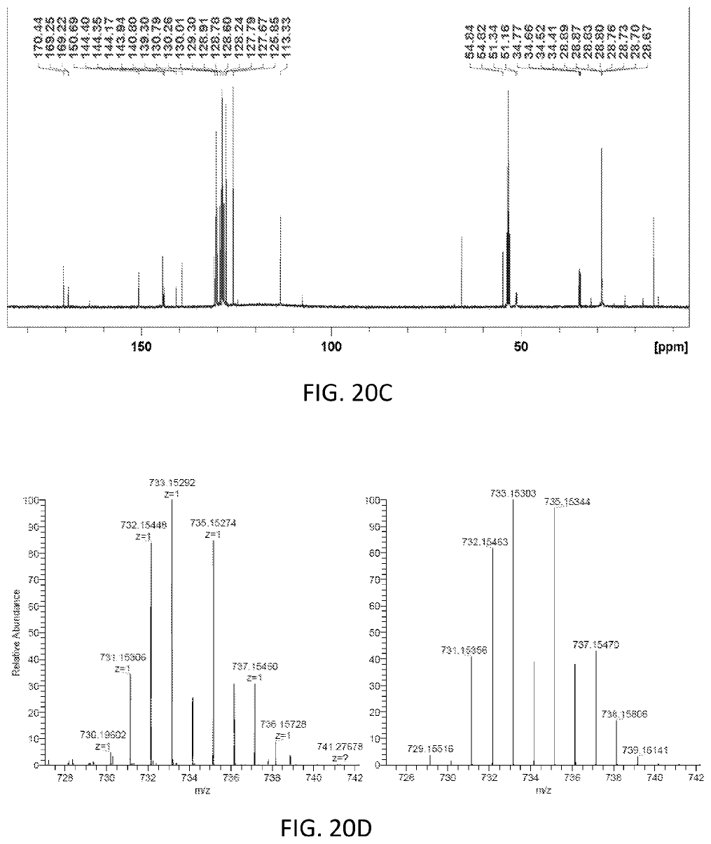

BippyPhos-Pd-MAH (catalyst 12): Prepared according to the general procedure using complex 1 (100.0 mg, 0.2133 mmol), BippyPhos (113.5 mg, 0.2240 mmol), and THE (5 mL). Trituration/decantation 5× with hexanes (5 mL). Tan solid: 130.0 mg (86%). ¹H NMR: (300 MHz, CDCl₃) δ 0.73 (d, 9H, J=15.0 Hz), 0.87 (d, 9H, J=15.3 Hz), 3.98 (dd, 1H, J=4.2, 6.3 Hz) 4.32 (d, 1H, J=4.1 Hz), 6.60 (d, 1H, J=2.1 Hz), 7.21-7.42 (m, 13H), 7.74 (m, 2H), 8.12 (d, 1H, J=1.9 Hz); ¹³C{¹H} NMR: (125 MHz, CD₂Cl₂) δ 28.7 (d, J=8.3 Hz), 28.8 (d, J=8.3 Hz), 34.5 (d, J=13.6 Hz), 34.7 (d, J=13.3 Hz), 51.3 (d, J=14.2 Hz), 54.8 (d, J=2.2 Hz), 107.7, 113.3, 125.9, 127.7, 127.8, 128.2, 128.6, 128.8, 128.9, 129.3, 139.0, 130.3, 130.8, 139.3, 140.8, 144.1 (d, J=29.3 Hz), 144.4 (d, J=5.8 Hz), 150.7, 169.2 (d, J=3.3 Hz), 170.4; ³¹P{¹H} NMR: (121 MHz, CDCl₃) δ 46.5. HRMS (ESI) of [C₃₆H₃₇N₄O₃PPd·Na]⁺ (major isotopomer, Na⁺ adduct): 733.15303 (calc'd); 733.15292 (found). Spectra are shown in FIGS. 20A-20D.

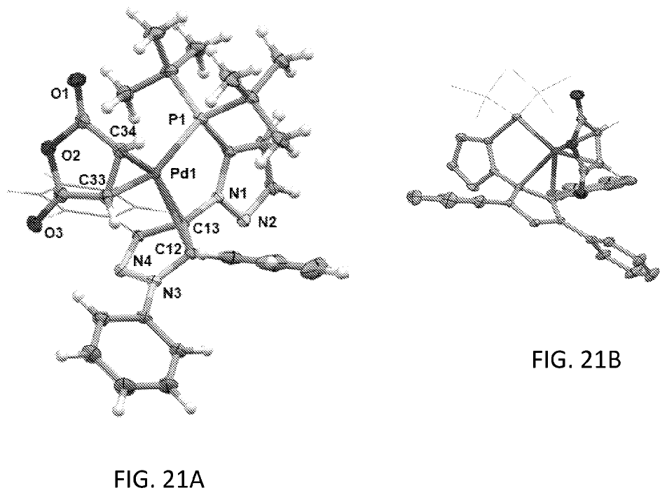

Single crystals of C_{39.5}H_{46.5}Cl₂N₄O_{3.5}PPd [catalyst 12·0.5Et₂O·DCM] were selected using a MitEGen loop using paratone oil. A suitable crystal was selected and run on a Bruker APEX-II CCD diffractometer. The crystal was kept at 100.0 K during data collection. Using Olex2, the structure was solved with the olex2.solve structure solution program using Charge Flipping and refined with the XL refinement package using Least Squares minimization. FIG. 21A shows the solid-state molecular structure of catalyst 12, including diethyl ether solvate and FIG. 21B shows the solid-state molecular structure determined by single crystal XRD wherein hydrogen atoms omitted for clarity, except those on the maleic anhydride ligand and phosphine substituents are shown in wireframe for clarity.

Crystal structure determination of [catalyst 12·0.5Et₂O-DCM] for C_{39.5}H_{46.5}Cl₂N₄O_{3.5}PPd (M=841.57 g/mol): monoclinic, space group P2₁/n (no. 14), a=12.848(2) Å, b=18.223(3) Å, c=16.659(3) Å, β=99.686(4°), V=3844.9(12) Å³, Z=4, T=100.0 K, μ(MoKα)=0.708 mm⁻¹, Dcalc=1.454 g/cm³, 35443 reflections measured (3.338°≤2Θ≤56.038°), 9241 unique (Rᵢₙₜ=0.0341, R_{sigma}=0.0344) which were used in all calculations. The final R₁ was 0.0393 (1>2σ(I)) and wR₂ was 0.1046 (all data).

### Example 8L

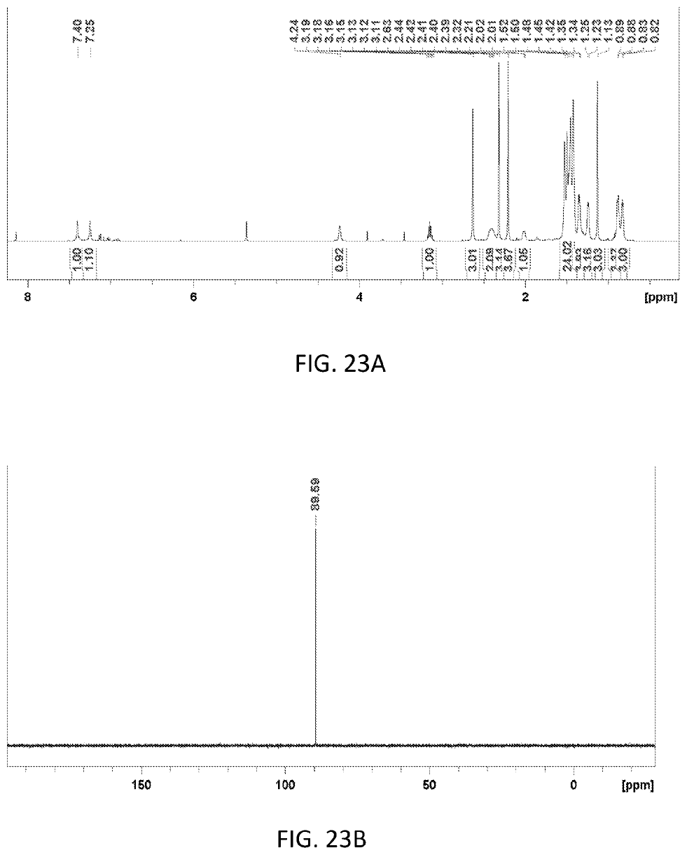

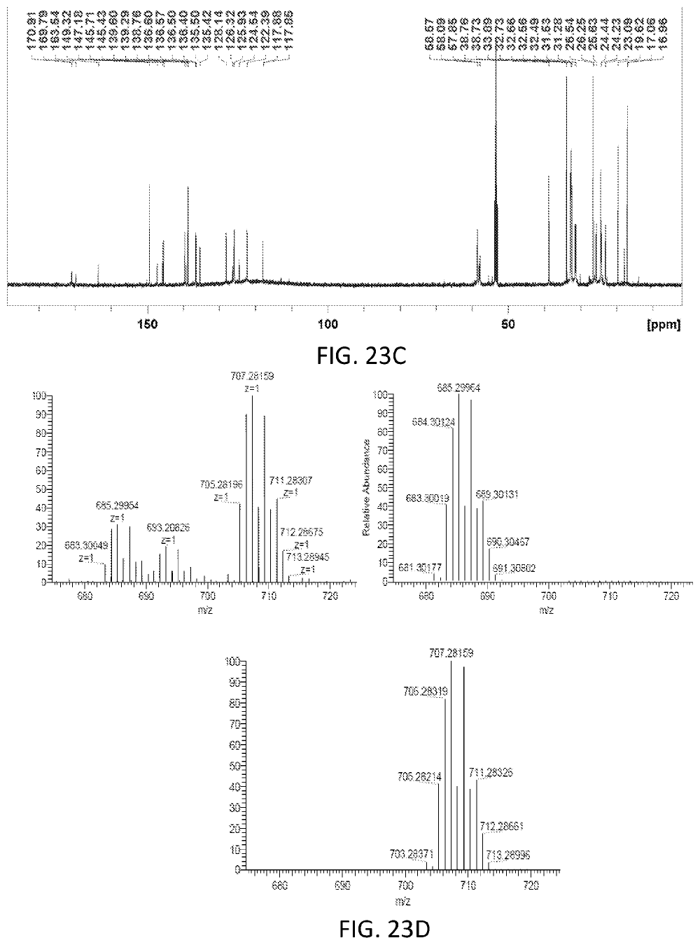

Me₄tBuXPhos-Pd-MAH (catalyst 13): Prepared according to the general procedure using complex 1 (101.8 mg, 0.2171 mmol), Me₄tBuXPhos (109.6 mg, 0.2126 mmol), and THE (11 mL). Trituration/decantation 7× with hexanes (5 mL). Yellow solid: 75.7 mg (51%). ¹H NMR: (300 MHz, C₆D₆) δ 0.53 (d, 9H, J=15.0 Hz), 0.77 (d, 9H, J=15.3 Hz), 1.41 (m, 4H, THF), 3.57 (m, 4H, THF), 3.89 (dd, 1H, J=4.2, 5.9 Hz) 4.11 (d, 1H, J=4.1 Hz), 6.07 (d, 1H, J=2.0 Hz), 6.80-7.02 (m, 8H), 7.06 (m, 2H), 7.41 (m, 2H), 7.76-7.86 (m, 5H); ¹³C{¹H} NMR: (125 MHz, CD₂Cl₂) δ 28.7 (d, J=8.3 Hz), 28.8 (d, J=8.3 Hz), 34.5 (d, J=13.6 Hz), 34.7 (d, J=13.3 Hz), 51.3 (d, J=14.2 Hz), 54.8 (d, J=2.2 Hz), 107.7, 113.3, 125.9, 127.7, 127.8, 128.2, 128.6, 128.8, 128.9, 129.3, 139.0, 130.3, 130.8, 139.3, 140.8, 144.1 (d, J=29.3 Hz), 144.4 (d, J=5.8 Hz), 150.7, 169.2 (d, J=3.3 Hz), 170.4; ³¹P{¹H} NMR: (121 MHz, C₆D₆) δ 46.6. Yield: 43%; ¹H NMR: (500 MHz, CD₂Cl₂) δ 0.83 (d, 3H, J=6.0 Hz), 0.88 (d, 3H, J=6.0 Hz), 1.13 (s, 3H), 1.24 (d, 3H, J=5.8 Hz), 1.35 (d, 3H, J=5.6 Hz), 1.38-1.60 (m, 24H), 2.01 (m, 1H), 2.21 (s, 3H), 2.21 (s, 3H), 2.40 (br m, 2H), 2.63 (s, 3H), 3.15 (sept, 1H, J=6.9 Hz), 4.24 (br s, 1H), 7.25 (s, 1H), 7.40 (s, 1H); ¹³C{¹H} NMR: (500 MHz, CD₂Cl₂) δ 17.0, 17.1, 19.6, 23.1, 24.2, 24.4, 25.6, 26.3, 26.5, 31.4 (d, J=30.2 Hz), 32.5, 32.6, 32.7, 32.7, 33.9, 38.7 (d, J=3.6 Hz), 58.0 (d, J=29.7 Hz), 58.6, 117.9 (d, J=4.5 Hz), 122.4, 124.5, 125.9, 126.3, 128.1, 135.5 (d, J=10.3 Hz), 136.4 (d, J=12.4 Hz), 136.6 (d, J=4.3 Hz), 138.8, 139.3, 139.6 (d, J=1.5 Hz), 145.6 (d, J=35.2 Hz), 147.2, 149.3, 163.5, 169.8; ³¹P{¹H} NMR: (300 MHz, C₆D₆) δ 89.6. HRMS (ESI) of [C₃₇H₅₅O₃PPd·H] (major isotopomer, H⁺ adduct): 685.29964 (calc'd); 685.29954 (found). Spectra are shown in FIGS. 23A-23D.

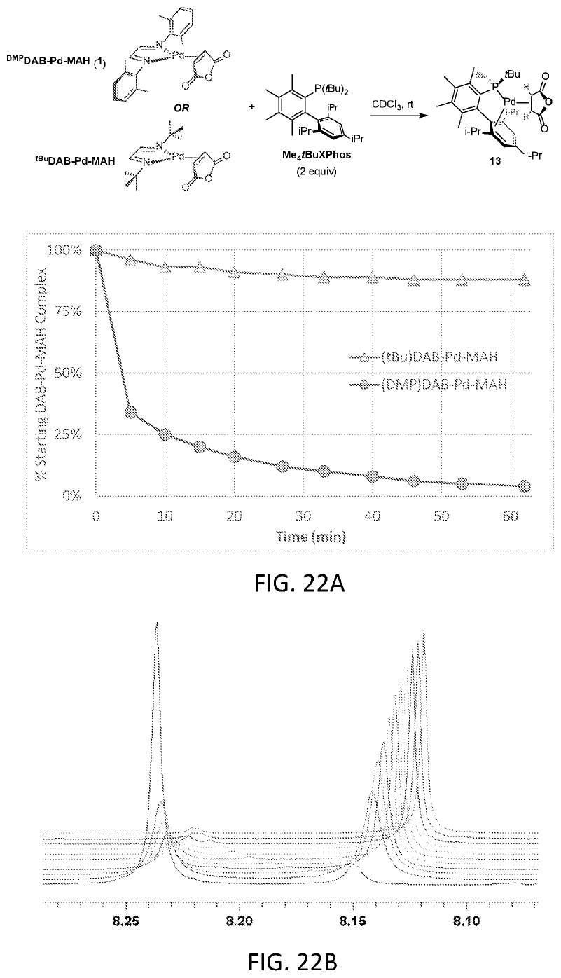

A comparison of ligand substitution reaction progress was assessed between ^{DMP}DAB-Pd-MAH (complex 1) or ^{tBu}DAB-Pd-MAH and Me₄tBuXPhos (2 equivalents) to generate catalyst 13. As shown by FIG. 22A, complex 1 is >95% converted after 60 min, whereas ˜90% of ^{tBu}DAB-Pd-MAH remains after the same time period. FIG. 22B shows an ¹H NMR spectrum stack plot of the ligand substitution between ^{DMP}DAB-Pd-MAH (1) (singlet at 8.24 ppm) and Me₄tBuXPhos (2 equivalents), with time increasing from front (0 minutes) to back (62 minutes). The singlet at 8.15 ppm is free ^{DMP}DAB (displaced by Me₄tBuXPhos).

### Example 8M

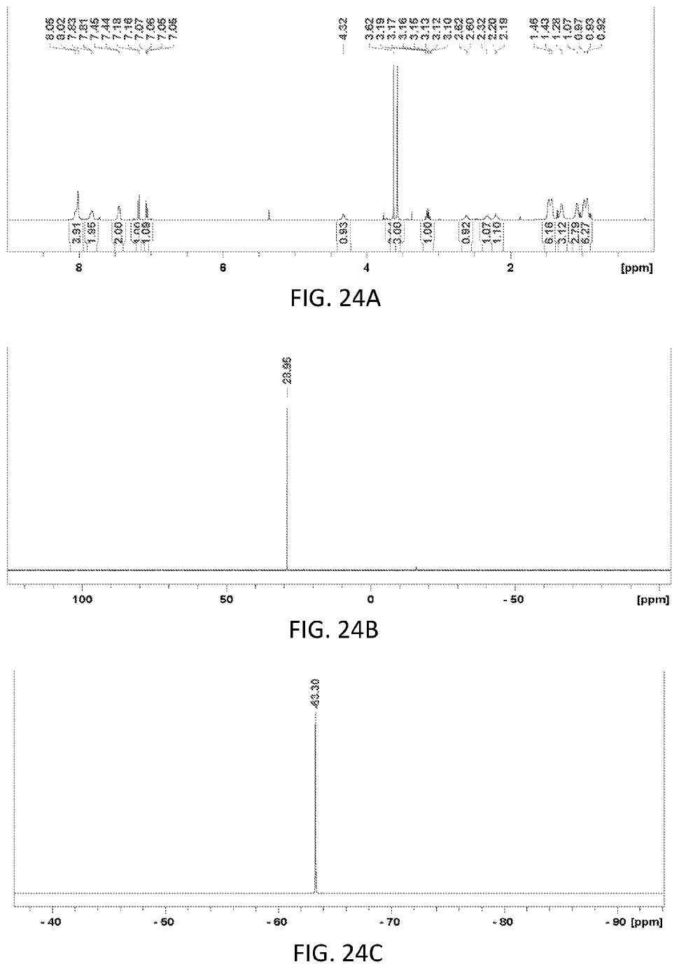

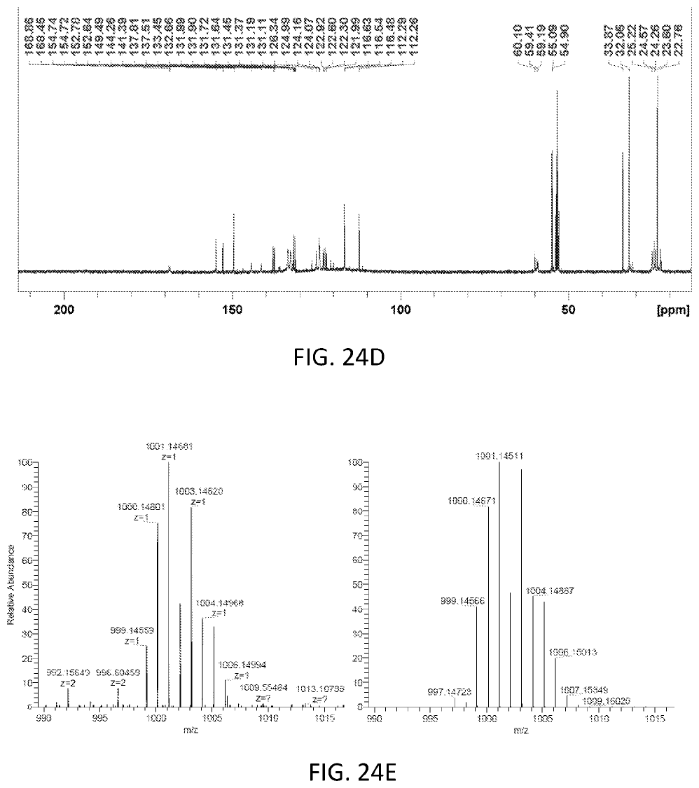

JackiePhos-Pd-MAH (catalyst 14): Prepared according to the general procedure using complex 1 (56.5 mg, 0.1205 mmol), JackiePhos (100.8 mg, 0.1265 mmol), and THF (4 mL). Trituration/decantation 6× with hexanes (4 mL). Yellow solid: 66.4 mg (55%). ¹H NMR: (500 MHz, CD₂Cl₂) δ 0.93 (br s, 3H), 0.97 (br s, 3H), 1.08 (br s, 3H), 1.29 (br s, 3H), 1.43 (br s, 3H), 1.46 (br s, 3H), 2.19 (Br s, 3H), 2.33 (br s, 1H), 2.26 (br d, 1H, J=6.9 Hz), 3.15 (sept, 1H, J=6.9 Hz), 3.57 (s, 3H), 3.62 (s, 3H), 4.32 (br s, 1H), 7.06 (dd, 1H, J=2.8 Hz, 9.0 Hz), 7.17 (d, 1H, J=8.9 Hz), 7.44 (br s, 1H), 7.46 (br s, 1H), 7.77-7.90 (m, 2H), 7.95-8.13 (m, 4H); ¹³C{¹H} NMR: (125 MHz, CD₂Cl₂) δ 22.8, 23.6, 24.3, 24.6, 25.2, 32.1, 33.9, 54.9, 55.1, 59.3 (d, J=29.3 Hz), 60.1, 112.3 (d, J=5.5 Hz), 116.5 (d, J=7.0 Hz), 116.6, 119.8, 120.6, 122, 122.4 (d, J=38.2 Hz), 122.9, 124.1, 124.2, 125.0, 126.3, 131.5 q (J=33.4 Hz), 131.6 q (J=33.5 Hz), 132.6, 133.5, 136.0 (d, J=34.9 Hz), 137.7 (d, J=38.0 Hz), 141.4, 144.3, 149.5, 152.7 (d, J=18.8 Hz), 154.7 (d, J=3.0 Hz), 168.5, 168.7; ³¹P{¹H} NMR: (202 MHz, CD₂Cl₂) δ 28.9; ¹⁹F{¹H} NMR: (282 MHz, CD₂Cl₂) δ 63.4. HRMS (ESI) of [C₄₃H₃₉F₁₂O₅PPd—H]⁺ (major isotopomer, H⁺ adduct): 1001.14511 (calc'd); 1001.14681 (found). Spectra are shown in FIGS. 24A-24E.

For this set of representative ligands, a single set of simple reaction conditions is sufficient to ensure complete metallation at room temperature, even for very large phosphines. This aspect of the reactivity of complex 1 makes it a potentially attractive Pd(0) precursor for HTE. One specific case is illustrative regarding the ability of complex 1 to undergo ligand substitution: Me₄tBuXPhos. This useful and sterically-demanding ligand is known to require elevated temperatures to react with Pd₂dba₃, and while Pd(II) precatalysts based on a palladacycle or allyl framework are known, in situ catalyst generation with this ligand is challenging. With 2 equivalents Me₄tBuXPhos and 1 equivalent of complex 1 in d₆-acetone, >95% conversion to catalyst 13 was observed after 60 min at room temperature. Thus, a simple room temperature pre-stir for ˜1 h is sufficient to generate catalyst 13 in situ for screening, as well as every other [phosphine]-Pd-MAH complex studied thus far.

### Example 8N

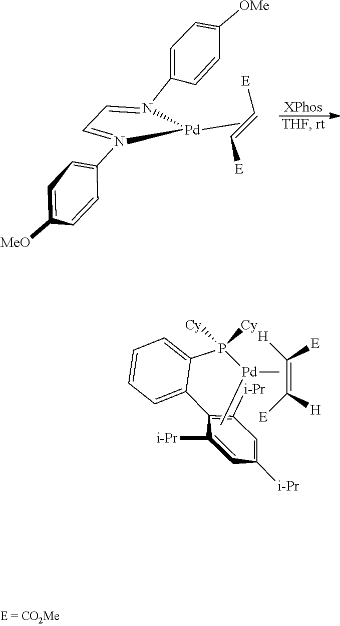

The reaction was conducted within a glovebox under a dry, oxygen-free N₂ atmosphere. A 100 mL round bottom flask was charged with bis(N,N′-bis(4-methoxyphenyl)ethane-1,2-diimine)(η2-trans-1,2-Ethylenedicarboxylic acid dimethyl ester)palladium(0) (100.0 mg, 0.193 mmol), XPhos (96.5 mg, 0.202 mmol), and 20 mL of tetrahydrofuran. The solution was stirred at room temperature for 1 hour, turning a clear dark amber-green soon after stirring began. The flask was then fitted with a vacuum-adapter and the solvent was evaporated off while stirring under vacuum, leaving a light green powder. To the flask, 15 mL of hexanes were added that had been freeze-pump-thawed before bringing into the glovebox, then the solution was decanted using a Pasteur pipette, leaving the displaced imine behind. The product was extracted 4 more times with 5 mL of hexanes, decanting by pipette each time. The hexanes solvent was then removed under vacuum. To extract the remaining imine, the product was stirred over 48 hours in 7 mL of hexanes.

The solution was filtered through a Pasteur pipette containing cotton and Celite, and the solvent extracted under vacuum. The final isolated yield of the orange crystals was 54.7 mg (39%). ¹H NMR (300 MHz, C6D6): δ 0.90 (d, 3H), 1.03 (d, 3H), 1.06-1.35 (m, 9H), 1.42 (d, 3H), 1.43 (d, 3H), 1.52 (d, 3H), 1.58 (d, 3H), 1.61-1.85 (m, 7H), 1.85-2.18 (m, 4H), 2.30 (sept, 1H), 2.46 (sept, 1H), 3.12 (br s, 1H), 3.31 (sept, 1H), 3.42 (s, 6H), 4.99 (br s, 1H), 6.85 (dd, 1H), 6.95 (tt, 1H), 7.02 (td, 1H), 7.29 (m, 1H), 7.32 (d, 1H), 7.47 (d, 1H). ³¹P{¹H} NMR (121 MHz, C₆D₆): δ 34.7.

### Example 80

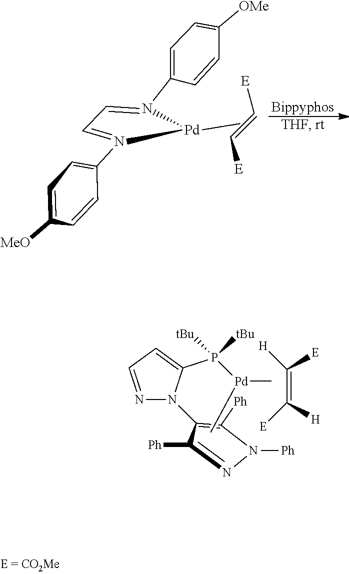

The reaction was conducted within a glovebox under a dry, oxygen-free N₂ atmosphere. A 100 mL round bottom flask was charged with bis(N,N′-bis(4-methoxyphenyl)ethane-1,2-diimine)(η2-trans-1,2-Ethylenedicarboxylic acid dimethyl ester)palladium(0) (35.6 mg, 0.0686 mmol), Bippyphos (36.5 mg, 0.0720 mmol), and 5 mL of tetrahydrofuran. The solution was stirred at room temperature for 3 hours. The resulting suspension was filtered to remove insoluble material, and the filtrate concentrated under vacuum. The solid residue was washed with hexanes (3×10 mL) and dried under vacuum. The final isolated yield of the tan-colored product was 29.7 mg (57%). ¹H NMR (300 MHz, CD₂Cl₂): δ 0.56 (d, 9H), 0.91 (d, 9H), 3.44 (s, 6H), 4.99 (br s, 1H), 6.52 (d, 1H), 7.10-7.35 (m, 13H), 7.65 (d, 2H), 7.95 (d, 1H), fumarate vinyl hydrogens not observed. ³¹P{¹H} NMR (121 MHz, CD₂Cl₂): δ 43.6.

### Example 9

In this example, certain complex embodiments were used for high-throughput screens. The traditional method to optimize the catalyst for a reaction is conducting a small quantity of millimole-scale reactions limited by one factor changed at one time. Instead of such an inefficient way, high-throughput experimentation was applied to determine catalytic conditions of several known palladium-catalyzed cross-coupling reactions with the ^{DMP}DAB-Pd-MAH catalyst precursor disclosed herein and three common palladium sources, including Pd(OAc)₂, [Pd(allyl)Cl]₂, and Pd₂dba₃·CHCl₃, as precatalysts.

Preparation of palladium sources: Four different palladium precatalysts were dissolved in corresponding solvents with high solubility in four different vials. 21.0 mg (0.0936 mmol) of palladium acetate was dissolved in 1348 μL of dichloromethane. 17.1 mg (0.0468 mmol) of allylpalladium(II) chloride dimer was dissolved in 2269 μL of acetone. 48.4 mg (0.0468 mmol) of recrystalized Pd₂dba₃·CHCl₃ was dissolved in 1968 μL of tetrahydrofuran. 43.9 mg of ^{DMP}DAB-Pd-MAH (0.0936 mmol) was dissolved in 2235 μL of acetone. These palladium sources stock solutions were dispensed into a 96-well-plate (24 wells for each palladium source). 40 uL of palladium acetate stock solution was dispensed into each of the 24 wells. 63.3 uL of allylpalladium chloride dimer stock solution was dispensed into each of the 24 wells. 56.3 uL of Pd₂dba₃·CHCl₃ stock solution was dispensed into each of the 24 wells. 63.3 uL of ^{DMP}DAB-Pd-MAH stock solution was dispensed into each of the 24 wells. The solvents were then evaporated through using Genevac. The 96-well-plate was stored in the glovebox for the further use.

Preparation of phosphine ligands: The ratio between the Pd loading and phosphine ligands is 2:1. Inside the glovebox, 0.0624 mmol of each phosphine ligand (33.5 mg of BrettPhos, 29.7 mg of XPhos, 26.5 mg of tBuXPhos, 30.2 mg tBuBrettPhos, 29.1 mg of RuPhos, 29.2 μL of P(tBu)₃) was dissolved in 600 μL of THE in 4 dram vials separately. 100 μL of each ligand stock solution was weighed, and the mass was recorded. 0.0052 mmol of each ligand stock solution (51.5 μL of BrettPhos stock solution, 53.3 μL of XPhos stock solution, 51.9 μL of tBuXPhos stock solution, 51.7 μL of tBuBrettPhos stock solution, 51.5 μL of RuPhos stock solution and 52.4 μL of P(tBu)₃) was dispensed into each of four wells containing four different palladium sources.

Preparation of reaction stock solution: A vial was in charge with 67.9 μL (0.72 mmol) of 2-chloropyridine, 78.8 μL (0.72 mmol) of benzylamine, 207.6 (2.16 mmol) mg of NaO-tBu, 36.3 mg (0.216 mmol) of 1,3,5-trimethoxybenzene, 5.4 mL of THE and a stir bar. After being mixed well, 161.1 μL of this reaction stock solution was added into each well. The top of 24-well-plate was screwed on, and the whole plate was taken out form the glovebox. The plate was placed on the hot plate and stirred for 3 hours at 80° C.

### Example 9A

N-Benzylpyridin-2-amine: A 4-dram vial was charged with 2-chloropyridine (426 μL, 4.50 mmol), benzylamine (590 μL, 5.40 mmol), NaOt-Bu (519 mg, 5.40 mmol), complex 1 (5.3 mg, 0.0113 mmol), BrettPhos (12.1 mg, 0.0225 mmol), and 9 mL of THF. The mixture was stirred at 60° C. for 1 h. Ethyl acetate was added to dilute the reaction mixture, and the diluted mixture was washed three times with water, dried over Mg₂SO₄ anhydrous and concentrated in the rotary evaporator. Finally, the crude product was dried in the vacuo to give 755 mg (4.10 mmol, 91%) product as a light yellow solid. ¹H NMR (300 MHz, CDCl₃, δ): 8.11 (d, J=4.0 Hz, 1H), 7.38 (m, 5H), 7.27 (m, 1H), 6.59 (m, 1H), 6.38 (d, J=8.4 Hz, 1H), 4.92 (br, 1H), 4.51 (d, J=5.8 Hz, 2H). ¹³C NMR (300 MHz, CDCl₃, δ): 158.7, 148.2, 139.2, 137.5, 128.6, 127.4, 127.2, 113.1, 106.8, 46.3.

Results from examples comparing a precatalyst embodiment (DAB-Pd-MAH) of the present disclosure with three other Pd sources (Pd(OAc)₂, [Pd(allyl)Cl]₂, and Pd₂dba₃·CHCl₃) are summarized in Table 2 below. The table provides the values for normalized HPLC product peak area/internal standard peak area. Different phosphine ligands also were used. Schemes also are provided summarizing conditions used for different embodiments.

TABLE 2
Conditions for Entries 2-6

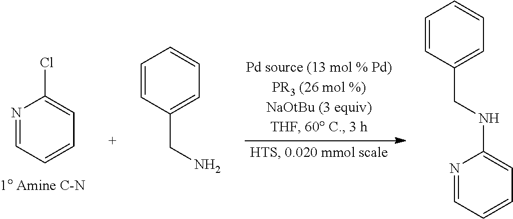 [SMILES: Clc1ccccn1 ; NCc1ccccc1 ; c1ccc(CNc2ccccn2)cc1]

Conditions for Entry 1

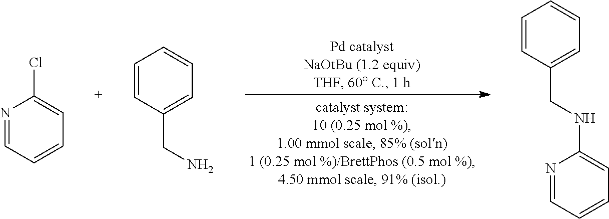 [SMILES: Clc1ccccn1 ; NCc1ccccc1 ; c1ccc(CNc2ccccn2)cc1]

| | | Pd(OAc)₂ | [Pd(allyl)Cl]₂ | Pd₂dba₃•CHCl₃ | DAB-Pd-MAH |
|---|---|---|---|---|---|
| 1 | BrettPhos | 0.63 | 0.57 | 0.56 | 0.98 |
| 2 | XPhos | 0.02 | 0.00 | 0.02 | 0.02 |
| 3 | tBuXPhos | 0.50 | 0.70 | 0.66 | 0.99 |
| 4 | tBuBrettPhos | 0.51 | 0.63 | 0.66 | 1.00 |
| 5 | RuPhos | 0.39 | 0.39 | 0.43 | 0.73 |
| 6 | P(tBu)₃ | 0.00 | 0.12 | 0.00 | 0.05 |

### Example 9B

4-(4-Methoxyphenyl)morpholine: A 4-dram vial was charged with 4-bromoanisole (128 μL, 1.00 mmol), morpholine (106 μL, 1.20 mmol), NaOt-Bu (116 mg, 1.20 mmol), complex 1 (9.4 mg, 0.02 mmol), RuPhos (18.7 mg, 0.04 mmol), and 2 mL of THF. The mixture was stirred at 80° C. for 2 h. Ethyl acetate was added to dilute the reaction mixture, and the diluted mixture was washed three times with water, dried over Mg₂SO₄ anhydrous and concentrated in the vacuo overnight to give 155.1 mg (0.92 mmol, 92%) product as a yellow solid. ¹H NMR (300 MHz, CDCl₃, δ): 6.87 (m, 4H), 3.84 (appt, J=4.8 Hz, 4H), 3.77 (s, 3H), 3.04 (appt, J=4.8 Hz, 4H). ¹³C NMR (300 MHz, CDCl₃, δ): 154.0, 145.6, 117.8, 114.5, 67.0, 55.5, 50.8.

Results from examples comparing a precatalyst embodiment (DAB-Pd-MAH) of the present disclosure with three other Pd sources (Pd(OAc)₂, [Pd(allyl)Cl]2, and Pd₂dba₃·CHCl₃) are summarized in Table 3 below. The table provides the values for normalized HPLC product peak area/internal standard peak area. Different phosphine ligands also were used. Schemes also are provided summarizing conditions used for different embodiments.

TABLE 3
Conditions for Entries 1-4 and 6

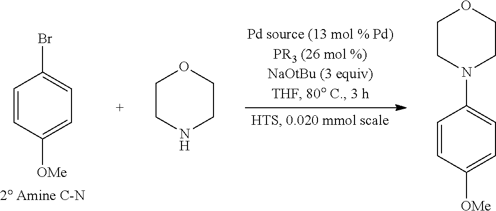 [SMILES: COc1ccc(Br)cc1 ; C1COCCN1 ; COc1ccc(N2CCOCC2)cc1]

Conditions for Entry 5

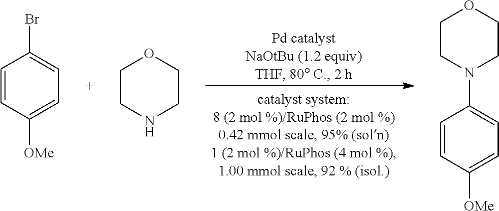 [SMILES: COc1ccc(Br)cc1 ; C1COCCN1 ; COc1ccc(N2CCOCC2)cc1]

| | | Pd(OAc)₂ | [Pd(allyl)Cl]₂ | Pd₂dba₃•CHCl₃ | DAB-Pd-MAH |
|---|---|---|---|---|---|
| 1 | BrettPhos | 0.66 | 0.79 | 0.76 | 0.81 |
| 2 | XPhos | 0.83 | 0.88 | 0.84 | 0.88 |
| 3 | tBuXPhos | 0.52 | 0.88 | 0.77 | 0.93 |
| 4 | tBuBrettPhos | 0.00 | 0.55 | 0.10 | 0.52 |
| 5 | RuPhos | 0.85 | 0.87 | 0.85 | 1.00 |
| 6 | P(tBu)₃ | 0.80 | 0.72 | 0.68 | 0.75 |

### Example 9C

N-(4-Methylphenyl)-4-methylbenzenesulfonamide: A 4-dram vial was charged with 4-bromotoluene (205 mg, 1.20 mmol), p-toluenesulfonamide (172 mg, 1.00 mmol), K₂CO₃ (415 mg, 3.00 mmol), complex 1 (23.4 mg, 0.05 mmol), tBuXPhos (42.5 mg, 0.10 mmol), and 4 mL of CPME. The mixture was stirred at 100° C. for 16 h. Ethyl acetate was added to dilute the reaction mixture, and the diluted mixture was washed three times with water, dried over MgSO₄ anhydrous and concentrated in the Genevac. The crude product was purified via a silica plug (DCM/TBME, 20:1) and dried over the Na₂SO₄ to give 183.7 mg (0.70 mmol, 70%) sulfate product as a pale yellow powder. ¹H NMR (300 MHz, CDCl₃, 6): 2.27 (s, 3H), 2.40 (s, 3H), 6.31 (br, 1H), 6.91 (d, J=8.4 Hz, 2H), 7.04 (d, J=8.3 Hz, 2H), 7.21 (d, J=8.0 Hz, 2H), 7.61 (d, J=8.3 Hz, 2H); ¹³C NMR (300 MHz, CDCl₃, δ): 143.7, 136.0, 135.0, 134.0, 129.6, 127.3, 122.0, 21.5, 20.8.

Results from examples comparing a precatalyst embodiment (DAB-Pd-MAH) of the present disclosure with three other Pd sources (Pd(OAc)₂, [Pd(allyl)Cl]2, and Pd₂dba₃·CHCl₃) are summarized in Table 4 below. The table provides the values for normalized HPLC product peak area/internal standard peak area. Different phosphine ligands also were used. Schemes also are provided summarizing conditions used for different embodiments. Additional comparisons and results are provided by Scheme 9C.

TABLE 4
Conditions for Entries 1, 2, and 4-6

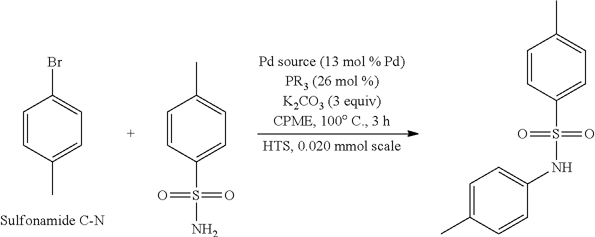

Conditions for Entry 3

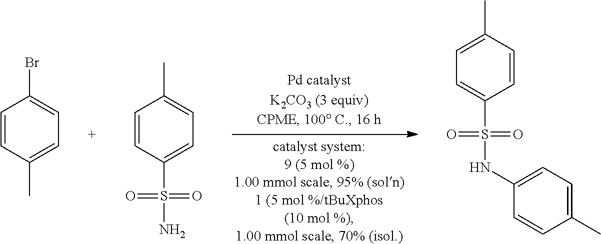

| | | Pd(OAc)₂ | [Pd(allyl)Cl]₂ | Pd₂dba₃•CHCl₃ | DAB-Pd-MAH |
|---|---|---|---|---|---|
| 1 | Xantphos | 0.07 | 0.17 | 0.12 | 0.16 |
| 2 | XPhos | 0.35 | 0.64 | 0.71 | 0.26 |
| 3 | tBuXPhos | 0.19 | 0.52 | 0.66 | 1.00 |
| 4 | Me₄tBuXPhos | 0.00 | 0.83 | 0.77 | 0.67 |
| 5 | Bippyphos | 0.18 | 0.75 | 0.83 | 0.40 |
| 6 | JackiePhos | 0.19 | 0.77 | 0.74 | 0.46 |

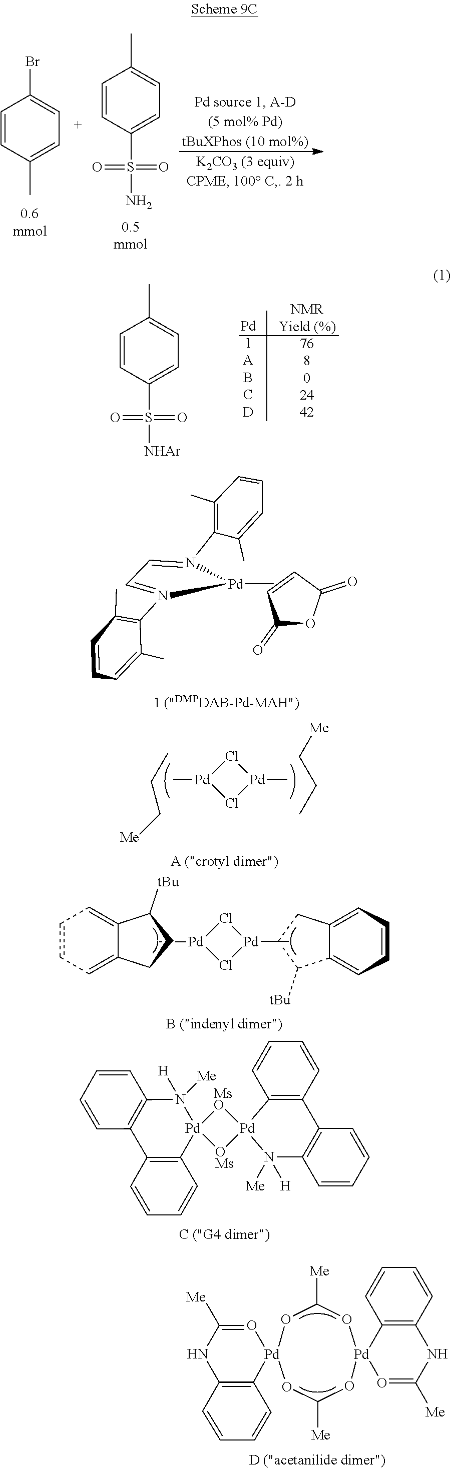

### Example 9D

4-Methyl-1-phenyl-1H-imidazole: A 4-dram vial was charged with bromobenzene (106 μL, 1.00 mmol), 4-methylimidazole (99 mg, 1.20 mmol), K3PO4 (425 mg, 2.00 mmol), catalyst 13 (10.3 mg, 0.015 mmol), and 1 mL of CPME. The mixture was stirred at 120° C. for 5 h. The mixture was extracted by DCM and concentrated by the Genevac. The crude product was purified via flash chromatography (ethyl acetate/hexane, 1:1) to give 101.1 mg (0.64 mmol, 64%) product as a pale yellow solid. ¹H NMR (300 MHz, CDCl3, δ): 7.76 (d, J=1.6 Hz, 1H), 7.49-7.44 (m, 2H), 7.37-7.31 (m, 3H), 7.01 (s, 1H), 2.30 (s, 3H); ¹³C NMR (300 MHz, CDCl₃, δ): 139.5, 137.4, 134.5, 129.8, 127.0, 121.0, 114.5, 13.7.

Results from examples comparing a precatalyst embodiment (DAB-Pd-MAH) of the present disclosure with three other Pd sources (Pd(OAc)₂, [Pd(allyl)Cl]2, and Pd₂dba₃·CHCl₃) are summarized in Table 5 below. The table provides the values for normalized HPLC product peak area/internal standard peak area. Different phosphine ligands also were used. Schemes also are provided summarizing conditions used for different embodiments.

TABLE 5
Conditions for Entries 1-3 and 6

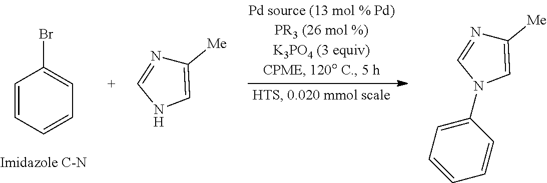 [SMILES: Brc1ccccc1 ; Cc1c[nH]cn1 ; Cc1cn(-c2ccccc2)cn1]

Conditions for Entries 4 and 5

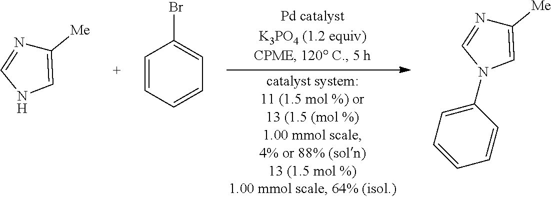 [SMILES: Cc1c[nH]cn1 ; Brc1ccccc1 ; Cc1cn(-c2ccccc2)cn1]

| | | Pd(OAc)₂ | [Pd(allyl)Cl]₂ | Pd₂dba₃•CHCl₃ | DAB-Pd-MAH |
|---|---|---|---|---|---|
| 1 | BrettPhos | 0.00 | 0.03 | 0.04 | 0.16 |
| 2 | XPhos | 0.00 | 0.00 | 0.00 | 0.10 |
| 3 | tBuXPhos | 0.02 | 0.00 | 0.06 | 0.36 |
| 4 | tBuBrettPhos | 0.01 | 0.30 | 0.74 | 1.00 |
| 5 | Me₄tBuXPhos | 0.00 | 0.26 | 0.48 | 0.35 |
| 6 | Bippyphos | 0.00 | 0.00 | 0.00 | 0.11 |

### Example 9E

(E)-3-(4-Acetylphenyl)-2-methyl Acrylic Acid Methyl Ester: A 4-dram vial was charged with 4-bromoacetophenone (185 mg, 0.93 mmol), methyl methacrylate (300 μL, 2.78 mmol), Cy₂NMe (220 μL, 1.03 mmol), complex 1 (4.5 mg, 0.0095 mmol), P(tBu)₃ (4.5 μL, 0.0095 mmol) and 0.84 mL of CPME. The mixture was stirred at 80° C. for 26 h. The mixture was extracted by DCM and washed by 1 M HCl and water, followed by the drying over the MgSO₄. The filtrate was concentrated by the Genevac to give 142.5 mg (0.65 mmol, 76%) product as a light brown solid with 15% side product from the second arylation. ¹H NMR (300 MHz, CDCl₃, 6): 7.98 (d, J=8.4 Hz, 2H), 7.70 (apparent s, 1H), 7.46 (d, J=8.3 Hz, 2H), 3.83 (s, 3H), 2.62 (s, 3H), 2.12 (d, J=1.5 Hz, 3H). ¹³C NMR (CDCl3, 75 MHz): δ 197.3, 168.6, 140.4, 137.5, 136.3, 130.3, 129.6, 128.3, 52.2, 26.6, 14.1.

Results from examples comparing a precatalyst embodiment (DAB-Pd-MAH) of the present disclosure with three other Pd sources (Pd(OAc)₂, [Pd(allyl)Cl]2, and Pd₂dba₃·CHCl₃) are summarized in Table 6A below. The table provides the values for normalized HPLC product peak area/internal standard peak area. Different phosphine ligands also were used. Additionally, Table 6B provides a summary of results obtained from using catalyst 11 in a 1.00 mmol scale reaction.

Schemes also are provided summarizing conditions used for different embodiments.

TABLE 6A
Conditions for Entries 1-5

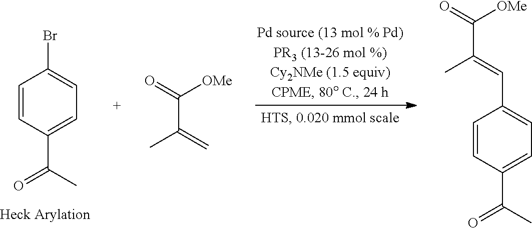

Conditions for Entry 6

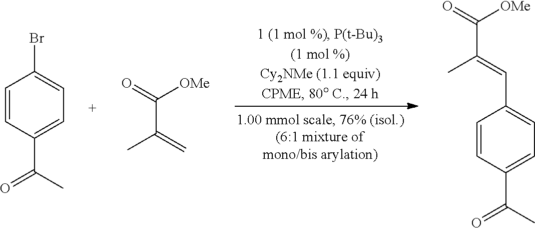

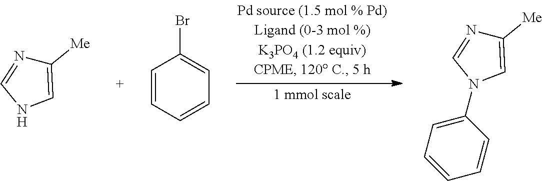 [SMILES: Cc1c[nH]cn1 ; Brc1ccccc1 ; Cc1cn(-c2ccccc2)cn1]

| | | Pd(OAc)₂ | [Pd(allyl)Cl]₂ | Pd₂dba₃•CHCl₃ | DAB-Pd-MAH |
|---|---|---|---|---|---|
| 1 | PCy₃ | 0.00 | 0.00 | 0.00 | 0.00 |
| 2 | XPhos | 0.26 | 0.00 | 0.25 | 0.00 |
| 3 | P(o-tol)₃ | 0.00 | 0.37 | 0.62 | 0.29 |
| 4 | cataCXium A | 0.00 | 0.00 | 0.00 | 0.00 |
| 5 | dppp | 0.15 | 0.00 | 0.00 | 0.00 |
| 6 | P(tBu)₃ | 0.00 | 1.00 | 0.99 | 0.68 |

TABLE 6B
Comparison of catalyst systems identified by
HTE for imidazole arylation on 1 mmol scale.

| Entry | Pd source | Ligand (mol %) | Yield (%)^{a} |
|---|---|---|---|
| 1 | 1 | tBuXPhos (3) | 1 |
| 2 | 1 | tBuXPhos (1.8) | 1 |
| 3 | 9 | n/a | 4 |
| 4 | Pd₂dba₃•CHCl₃ | tBuBrettPhos (1.8) | 6 |
| 5 | 1 | tBuBrettPhos (1.8) | 3 |
| 6 | 11 | n/a | 4 |
| 7 | Pd₂dba₃•CHCl₃ | Me₄tBuXPhos (3) | 40 |
| 8 | Pd₂dba₃•CHCl₃ | Me₄tBuXPhos (1.8) | 5 |
| 9 | 1 | Me₄tBuXPhos (3) | 39 |
| 10 | 1 | Me₄tBuXPhos (1.8) | 36 |
| 11 | A^{c} | Me₄tBuXPhos (1.8) | 1 |
| 12 | B^{c} | Me₄tBuXPhos (1.8) | 12 |
| 13 | C^{c} | Me₄tBuXPhos (1.8) | 0 |
| 14 | D^{c} | Me₄tBuXPhos (1.8) | 0 |
| 15 | 13 | n/a | 88, 64^{d} |

^{a}Determined by ¹H NMR spectroscopy versus internal standard (1,3,5-trimethoxybenzene).
^{b}Isolated yield of a separate experiment after chromatography.
^{c}Palladium sources A-D are as illustrated above in Scheme 9C.
^{d}Isolated yield of a separate experiment after chromatography.

### Example 9F

1-(4-n-Butoxyphenyl)ethenone: A 4-dram vial was charged with 4-bromoacetophenone (199 mg, 1.00 mmol), butyl alcohol (275 μL, 3.00 mmol), Cs₂CO₃ (489 mg, 1.50 mmol), complex 1 (4.7 mg, 0.01 mmol), tBuBippyPhos (10.1 mg, 0.02 mmol), and 1.8 mL of CPME. The mixture was stirred at 80° C. for 18 h. The mixture was extracted by ethyl acetate and washed three times with water. The rag layer was combined with the aqueous phase and extracted by the ethyl acetate for two times. The organic layers were dried over Mg₂SO₄ and concentrated in Genevac to give 168.2 mg (0.87 mmol, 87%) product as a brown liquid. ¹H NMR (300 MHz, CDCl₃, δ): 7.92 (m, 2H), 6.91 (m, 2H), 4.02 (t, J=6.5 Hz, 2H), 2.55 (s, 3H), 1.78 (m, 2H), 1.50 (m, 2H), 0.98 (t, J=7.03 Hz, 3H). ¹³C NMR (300 MHz, CDCl₃, δ): 196.8, 163.1, 130.5, 130.0, 114.1, 67.9, 31.1, 26.3, 19.2, 13.8.

Results from examples comparing a precatalyst embodiment (DAB-Pd-MAH) of the present disclosure with three other Pd sources (Pd(OAc)₂, [Pd(allyl)Cl]₂, and Pd₂dba₃·CHCl₃) are summarized in Table 7 below. The table provides the values for normalized HPLC product peak area/internal standard peak area. Different phosphine ligands also were used. Schemes also are provided summarizing conditions used for different embodiments. Additional comparisons are summarized in Scheme 9F, wherein palladium sources A-D are as recited above in Example 9C.

TABLE 7
Conditions for Entries 1-5

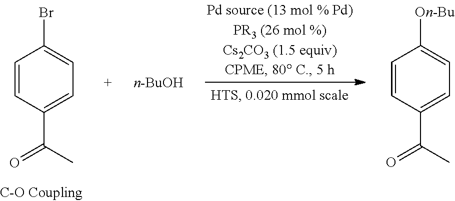

Conditions for Entry 6

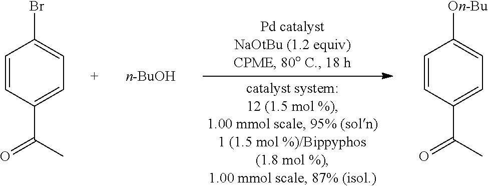

| | | Pd(OAc)₂ | [Pd(allyl)Cl]₂ | Pd₂dba₃•CHCl₃ | DAB-Pd-MAH |
|---|---|---|---|---|---|
| 1 | BrettPhos | 0.78 | 0.30 | 0.84 | 0.80 |
| 2 | XPhos | 0.14 | 0.28 | 0.14 | 0.12 |
| 3 | tBuXPhos | 0.49 | 0.20 | 0.67 | 0.48 |
| 4 | tBuBrettPhos | 0.67 | 0.25 | 0.67 | 0.60 |
| 5 | Me₄tBuXPhos | 0.35 | 0.37 | 0.47 | 0.42 |
| 6 | Bippyphos | 1.00 | 0.35 | 0.87 | 0.99 |

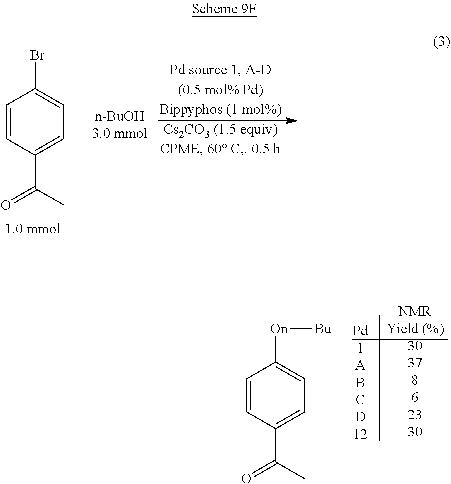

### Example 9G

2,2′,7,7′-Tetrakis-di(p-methoxyphenylamino)-9,9′-spirobifluorene (Spiro-OMeTAD)—A 4-dram vial was charged with 2,2′,7,7′-tetrabromo-9,9′-spirobifluorene (632 mg, 1.00 mmol), 4,4′-dimethoxydiphenylamine (940 mg, 4.10 mmol), NaOtBu (480.5 mg, 5.00 mmol), catalyst 6 (6.2 mg, 0.01 mmol), and 10 mL of THF. The mixture was stirred at 80° C. for 24 h. The solvent was evaporated in vacuo. The residue was dissolved in minimal toluene, and passed through a silica plug (toluene:TBME, 20:1 eluent). The resulting solution was concentrated to give an orange solid. The solid was dissolved in 5 mL of THF, followed by the addition of 30 mL of methanol (anti-solvent) to precipitate the product. Pale yellow solid: 994.4 mg (0.81 mmol, 84% yield). ¹H NMR (500 MHz, d⁶-DMSO): δ 3.72 (s, 24H, 8×—OCH₃), 6.19 (d, 4H, J=2.0 Hz), 6.70 (dd, 4H, J=2.0, 8.4 Hz), 6.84 (m, 32H, Ar—H), 7.48 (d, 4H, J=8.4 Hz).

Results from examples comparing a precatalyst embodiment (DAB-Pd-MAH) of the present disclosure with three other Pd sources (Pd(OAc)₂, [Pd(allyl)Cl]2, and Pd₂dba₃·CHCl₃) are summarized in Table 8 below. The table provides the values for normalized HPLC product peak area/internal standard peak area. Different phosphine ligands also were used.

TABLE 8

| | | | | DAB- |
|---|---|---|---|---|
| | Pd(OAc)₂ | [Pd(allyl)Cl]₂ | Pd₂dba₃•CHCl₃ | Pd-MAH |
| BrettPhos | 0.17 | 0.21 | 0.56 | 0.29 |
| XPhos | 0.77 | 0.72 | 0.98 | 1.00 |
| tBuXPhos | 0.27 | 0.46 | 0.52 | 0.49 |
| SPhos | 0.86 | 0.83 | 0.95 | 0.84 |
| RuPhos | 0.89 | 0.93 | 0.86 | 0.82 |
| XantPhos | 0.87 | 0.90 | 0.88 | 0.93 |

### Example 9H

In this example, complex 1 was evaluated for its use in the Mizoroki-Heck coupling. Reaction conditions and results are summarized below.

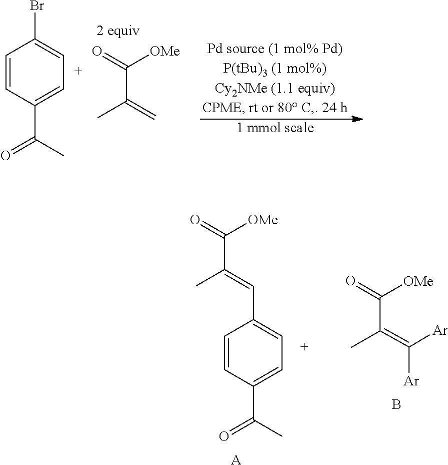

TABLE 9
Comparison of catalyst systems identified by HTE
for Mizoroki-Heck arylation on 1 mmol scale.

| Entry | Pd source | Temp. (° C.) | Yield A (%)^{a} | Yield B (%)^{a} |
|---|---|---|---|---|
| 1 | Pd₂dba₃•CHCl₃ | rt | 23 | 5 |
| 2 | Complex 1 | rt | 5 | n/d |
| 3 | Pd₂dba₃•CHCl₃ | 80 | 74 | 21 |
| 4 | Complex 1 | 80 | 74 | 14 |
| 5^{b} | Complex 1 | 80 | 76,^{c} A:B = 6:1^{d} | |

^{a}Determined by ¹H NMR spectroscopy versus internal standard (1,3,5-trimethoxybenzene); n/d = not detected.
^{b}3 equivalents methyl methacrylate.
^{c}Isolated yield based on combined mass of both products.
^{d}Ratio determined by ¹H NMR spectroscopy.

In view of the many possible embodiments to which the principles of the present disclosure may be applied, it should be recognized that the illustrated embodiments are only preferred examples and should not be taken as limiting the scope of the present disclosure. Rather, the scope is defined by the following claims. We therefore claim as our invention all that comes within the scope and spirit of these claims.

1. A complex, having a structure according to Formula III 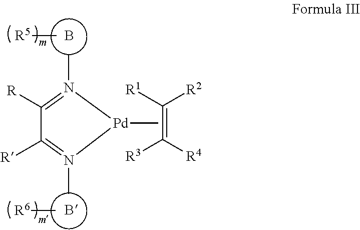
wherein
each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen;
each of R and R′ independently is selected from hydrogen, aliphatic, heteroaliphatic, aromatic, or an organic functional group, or R and R′ together to provide an aromatic, heterocyclic, or alicyclic ring system;
each of the B ring and B′ ring independently is an aromatic ring system;
each R⁵ independently is selected from aliphatic, heteroaliphatic, haloaliphatic, aromatic, an organic functional group, or a combination thereof;
each R⁶ independently is selected from aliphatic, heteroaliphatic, haloaliphatic, aromatic, an organic functional group, or a combination thereof; and
each of m and m′ independently is an integer selected from 0 to 10.

2. The complex of claim 1, wherein R¹ and R³, or R² and R⁴ join together to provide a 5-membered cyclic group.

3. The complex of claim 2, wherein the 5-membered cyclic group is maleic anhydride.

4. The complex of claim 1, wherein the B ring and the B′ ring are both phenyl.

5. The complex of claim 1, wherein each R⁵ is lower alkyl; lower haloalkyl; lower heteroalkyl; halide; cyano; ester; amide; or amine; and/or wherein each R⁶ is lower alkyl; lower haloalkyl; lower heteroalkyl; halide; cyano; ester; amide; or amine.

6-7. (canceled)

8. The complex of claim 1, wherein each of m and m′ independently is 2.

9. The complex of claim 1, wherein R and R′ are hydrogen or join together to provide an aromatic or alicyclic ring system.

10. The complex of claim 1, wherein the complex has a structure according to Formula IIIA, IIIB, IIIC, or IIID 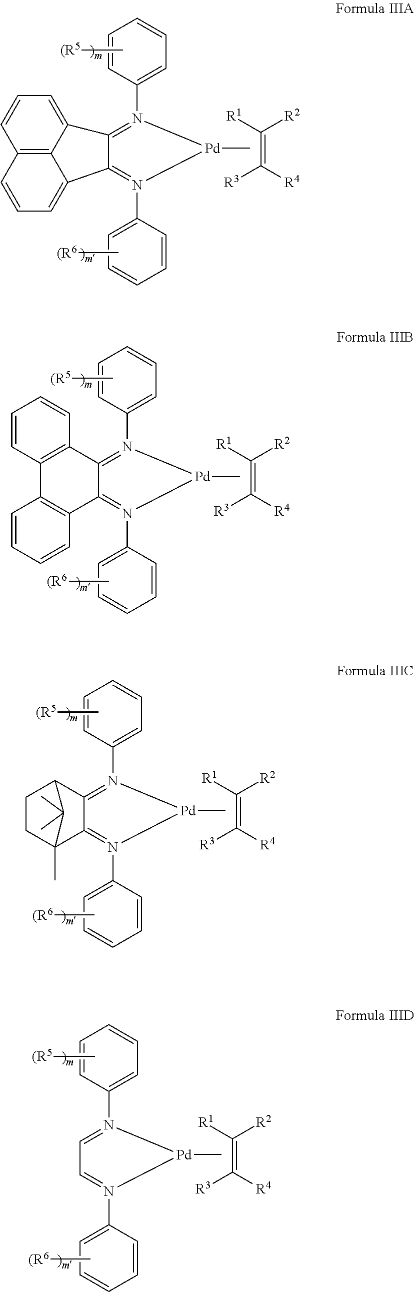
provided that, for Formula IIIA, if (i) the A group is maleic anhydride, (ii) m and m′ are 1, and (iii) R⁵═R⁶, then R⁵ and R⁶ are not, or are other than, OMe, methyl, isopropyl, chloro, or trifluoromethyl; or if (i) the A group is maleic anhydride, (ii) m and m′ are 2, and (iii) R⁵═R⁶, then R⁵ and R⁶ are not, or are other than, methyl, isopropyl, or trifluoromethyl.

11. The complex of claim 1, wherein the complex is selected from

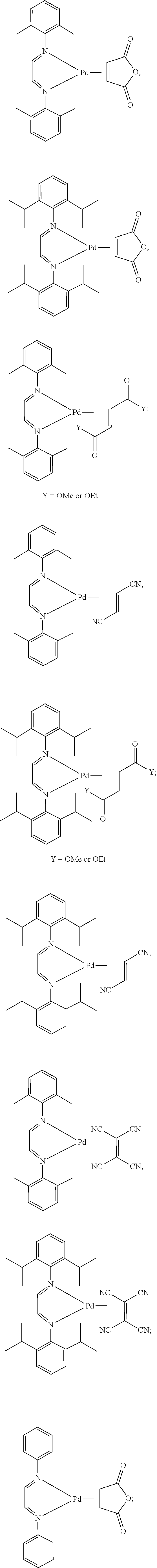

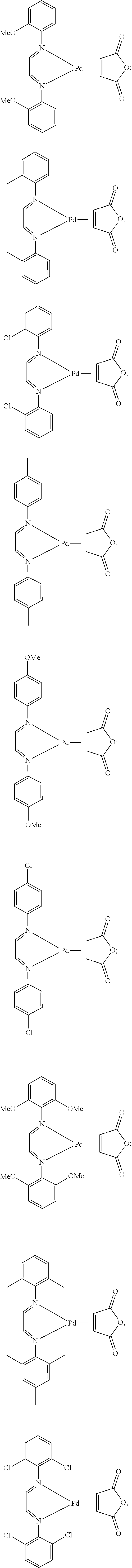

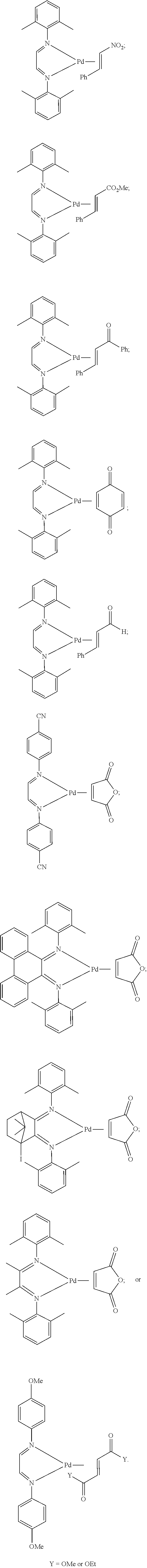

12. A catalyst, having a structure according to Formula IV, VA, or VB 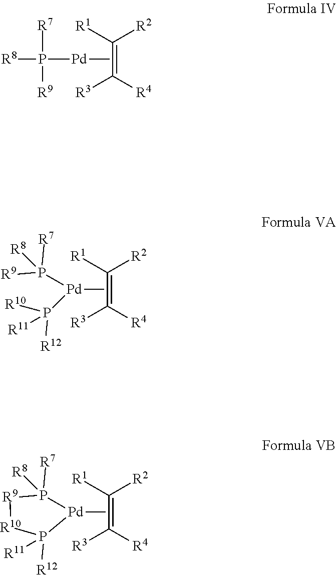
wherein with reference to Formulas IV and V,
each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; and
each of R⁷, R⁸, R⁹, R¹⁰, R¹¹, and R¹² independently is selected from aliphatic, heteroaliphatic, haloaliphatic, haloheteroaliphatic, aromatic, or an organic functional group; or two or more of R⁷, R⁸, and R⁹, and/or independently two or more of R¹⁰, R¹¹, and R¹², can join together, with the phosphorus atom to which they are attached, to provide a heterocyclic ring system; and
wherein with reference to Formula VB,
each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen; or
each of R⁷, R⁸, R¹¹, and R¹² independently is selected from aliphatic, heteroaliphatic, haloaliphatic, haloheteroaliphatic, aromatic, or an organic functional group and R⁹ and R¹⁰ independently are aliphatic, heteroaliphatic, haloaliphatic, haloheteroaliphatic, aromatic, or an organic functional group and are bound together through one or more carbon atoms and/or heteroatoms.

13. The catalyst of claim 12, wherein R¹ and R³, or R² and R⁴, join together to provide a 5-membered cyclic group.

14. The catalyst of claim 13 wherein the 5-membered cyclic group is maleic anhydride.

15. The catalyst of claim 12, wherein the catalyst has a structure according to:
Formula IV and each of R⁷ and R⁸ is phenyl, cyclohexyl, t-butyl, or 3,5-(CF₃)₂-phenyl, and R⁹ is selected from 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; or 2′,4′,6′-triisopropyl-2,3,4,5-tetramethyl-1,1′-biphenyl; or
Formula VA and each of R⁷, R⁸, R¹¹, and R¹² independently is phenyl or cyclohexyl and R⁹ is selected from 2,6-dimethoxy-1,1′-biphenyl; 2,6-diisopropoxy-1,1′-biphenyl; 2,4,6-triisopropyl-1,1′-biphenyl; 2,4,6-triisopropyl-2′,6′-dimethoxy-1,1′-biphenyl; 1′,3′,5′-triphenyl-1′H-1,4′-bipyrazole; or 2′,4′,6′-triisopropyl-2,3,4,5-tetramethyl-1,1′-biphenyl; or
Formula VA and each of R⁷, R⁸, R⁹, R¹⁰, R¹¹ and R¹² independently is aromatic or aliphatic.

16. The catalyst of claim 15, wherein the catalyst is selected from

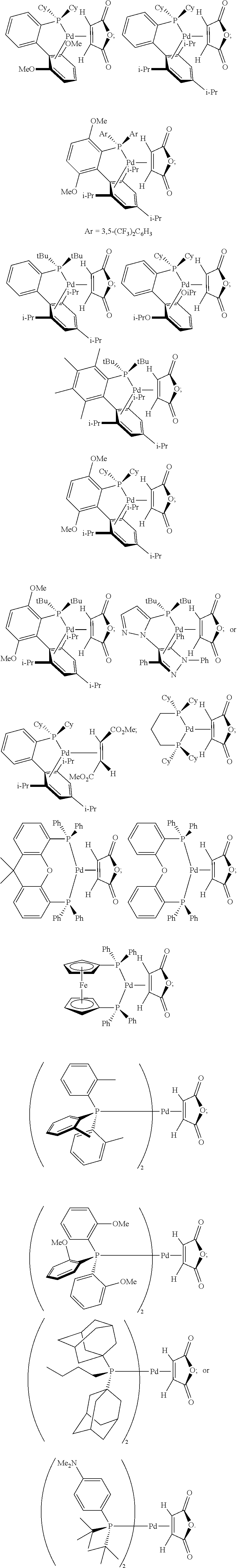

17-20. (canceled)

21. A method of making the complex of claim 1, comprising exposing a Pd₂dba₃ complex to a ligand compound comprising a N,N-substituted diazabutadiene group to form a reaction mixture.

22. The method of claim 21, wherein the ligand group comprising the N,N-substituted diazabutadiene group is N,N′-bis(2,6-dimethylphenyl)ethan-1,2-diimine.

23. The method of claim 21, wherein the method further comprises isolating the complex by exposing the reaction mixture to tert-butyl methyl ether, stirring the reaction mixture with the tert-butyl methyl ether to form a slurry; decanting the slurry to isolate a solid therefrom, and optionally filtering the solid to remove any palladium black.

24. A method of making the catalyst of claim 12, comprising exposing a phosphorus-containing ligand group to a complex having a structure according to Formula III 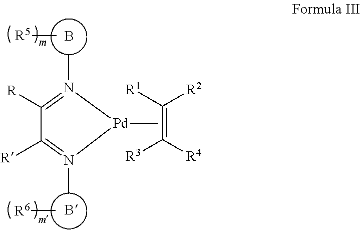
wherein
each of R¹, R², R³, and R⁴ independently is selected from hydrogen, aliphatic, or an electron-withdrawing group provided that at least one of R¹, R², R³, or R⁴ is other than hydrogen; or R¹ and R³, or R² and R⁴ join together to provide a cyclic group and the remaining R¹ and R³ groups, or R² and R⁴ groups are hydrogen;
each of R and R′ independently is selected from hydrogen, aliphatic, heteroaliphatic, aromatic, or an organic functional group, or R and R′ together to provide an aromatic, heterocyclic, or alicyclic ring system;
each of the B ring and B′ ring independently is an aromatic ring system;
each R⁵ independently is selected from aliphatic, heteroaliphatic, haloaliphatic, aromatic, an organic functional group, or a combination thereof;
each R⁶ independently is selected from aliphatic, heteroaliphatic, haloaliphatic, aromatic, an organic functional group, or a combination thereof; and
each of m and m′ independently is an integer selected from 0 to 10.

25. The method of claim 24, wherein the phosphorus-containing ligand group is selected from 1,3-bis(diphenylphosphino)propane, Bis[(2-diphenylphosphino)phenyl] ether, 4,5-Bis(diphenylphosphino)-9,9-dimethylxanthene, 1,1′-Bis(diphenylphosphino)ferrocene, 2-Dicyclohexylphosphino-2′,6′-dimethoxybiphenyl, 2-Dicyclohexylphosphino-2′,6′-diisopropoxybiphenyl, 2-Dicyclohexylphosphino-2′,4′,6′-triisopropylbiphenyl, 2-Di-tert-butylphosphino-2′,4′,6′-triisopropylbiphenyl, 2-(Dicyclohexylphosphino)3,6-dimethoxy-2′,4′,6′-triisopropyl-1,1′-biphenyl, 2-(Di-tert-butylphosphino)-2′,4′,6′-triisopropyl-3,6-dimethoxy-1,1′-biphenyl, 5-(Di-tert-butylphosphino)-1′, 3′, 5′-triphenyl-1′H-[1,4′]bipyrazole, 2-Di-tert-butylphosphino-3,4,5,6-tetramethyl-2′,4′,6′-triisopropyl-1,1′-biphenyl, 2-{Bis[3,5-bis(trifluoromethyl)phenyl]phosphino}-3,6-dimethoxy-2′,4′,6′-triisopropyl-1,1′-biphenyl, or Di(1-adamantyl)-n-butylphosphine.

26. A method, comprising using a complex according to claim 1, or a catalyst formed therefrom, as a catalyst in a palladium-mediated coupling reaction.
